(19) United States
(12) Patent Application Publication
(10) Pub. No.: US 2016/0030517 A1
(43) Pub. Date: Feb. 4, 2016

(54) COMPOSITIONS AND METHODS FOR DIAGNOSIS AND TREATMENT OF NEUROLOGICAL DISEASE

(71) Applicant: UNIVERSITY OF WASHINGTON THROUGH ITS CENTER FOR COMMERCIALIZATION, Seattle, WA (US)

(72) Inventors: Richard S. MORRISON, Seattle, WA (US); David Boyu WANG, Seattle, WA (US)

(73) Assignee: UNIVERSITY OF WASHINGTON THROUGH ITS CENTER FOR COMMERCIALIZATION, Seattle, WA (US)

(21) Appl. No.: 14/434,867

(22) PCT Filed: Oct. 10, 2013
(86) PCT No.: PCT/US13/64292

§ 371 Date: Apr. 10, 2015

Related U.S. Application Data
(60) Provisional application No. 61/712,130, filed on Oct. 10, 2012.

(51) Int. Cl. A61K 38/17; G01N 33/68

(52) U.S. Cl. CPC A61K 38/1712; G01N 33/6896; A61K 48/00

(57) ABSTRACT

Provided herein are methods, assays and compositions relating to the treatment of neurological diseases and disorders, particularly by modulating expression and/or activity of Bif-1.

### CROSS-REFERENCE TO RELATED APPLICATIONS

This application claims benefit under 35 U.S.C. §119(e) of U.S. Provisional Application No. 61/712,130, filed Oct. 10, 2012, the contents of which is incorporated herein by reference in its entirety.

### TECHNICAL FIELD

The technical field relates to the diagnosis and treatment of neurological disease.

### BACKGROUND

Bax-interacting factor-1 (Bif-1), also known as endophilin B1, was originally identified as a proapoptotic protein that binds to and activates Bax in response to apoptotic stress (1). Overexpression of Bif-1 promotes apoptosis (1), while knockdown of Bif-1 suppresses cytochrome c release and apoptosis (2). Consistent with the ability of endophilins to induce membrane curvature, Bif-1 has also been implicated in the regulation of mitochondrial morphology, as knockdown or expression of a dominant negative form of Bif-1 resulted in elongated mitochondria in HeLa cells (3).

Accumulating evidence suggests that a multitude of diseases are associated with dysfunctional regulation of mitochondrial dynamics (4). Changes in mitochondrial size and shape are regulated by the processes of fission and fusion, through the action of highly conserved dynamin-related GTPases, including dynamin-related protein 1 (Drp1) for fission and mitofusins (Mfn1 and Mfn2) for fusion (5). The size, shape, and distribution of mitochondria in neurons are especially important for neuronal survival and synaptogenesis (4, 6). For instance, knockout of Drp1 in mice results in abnormally large mitochondria that cannot be transported out of the soma into the processes, leading to Purkinje cell degeneration (7). Mitochondria in fibroblasts of these mice were normal, however, suggesting that neurons are uniquely vulnerable to changes in mitochondrial dynamics (7). Supporting these observations, loss-of-function mutations in dynamin-related GTPases often result in neurodegenerative phenotypes (8-10). The levels of expression and cellular distribution of these GTPases are also affected in Alzheimer's disease and Huntington's disease (11, 12), providing further support that alterations in mitochondrial dynamics may be causally related to neurodegeneration.

### SUMMARY

The methods, compositions, and assays described herein are based, in part, on the discovery that Bif-1 acts as an anti-apoptotic factor in neurons. That is, in contrast to the role of Bif-1 in non-neuronal cells, loss of Bif-1 expression in neurons results in increased cell death, particularly under conditions of stress (e.g., ischemic injury or other stress). The methods and assays described herein are directed to the treatment of neurological disease by increasing expression and/or activity of Bif-1 in neurons. In one embodiment, the methods provided herein relate to reducing amyloid beta-mediated neurotoxicity by increasing the expression and/or activity of Bif-1. Also provided herein are methods and assays directed to predicting sensitivity of a subject to neurological damage by measuring and/or quantifying the level of Bif-1 in a subject, either in vitro or in vivo.

The present disclosure relates to compositions and methods of use for Bax Interacting Factor-1 (Bif1). More particularly, the present disclosure relates to use of Bif1 as a biomarker in diagnosing, predicting, and/or detecting neurological damage. In addition, the present disclosure relates to compositions and methods of preventing, treating, and/or otherwise enhancing treatment of neurological damage using Bif1 and/or Bif1 Isoforms: Bif1A, Bif1B, and/or Bif1C.

In one aspect, the present disclosure is directed to methods for detecting, diagnosing, and/or predicting the occurrence, susceptibility, and/or seriousness of disorders, injuries, and/or diseases that cause and/or result in neurological damage such as neuronal cell death and/or degeneration and axonal injury. In some embodiments, these disorders, injuries, and/or diseases can include stroke, neurodegenerative diseases such as Alzheimer's disease (AD), Parkinson's disease, traumatic brain injury/head injury and others. These methods for detecting, diagnosing, and/or predicting the occurrence, susceptibility, and/or seriousness of disorders, injuries, and/or diseases that cause and/or result in neurological damage such as neuronal cell death and/or degeneration can comprise measuring and/or detecting the presence of Bif1 and/or Bif1 isoforms in a subject. The Bif1 and/or Bif1 isoforms can be detected in bodily fluid, such as but not limited to blood, cerebral spinal fluid (CSF), urine, saliva, and so forth. Bif1 isoforms can include Bif1A, Bif1B, and/or Bif1C or other novel Bif-1 isoforms that exist as currently unidentified mRNA transcripts.

As the present disclosure indicates, isoforms of Bif1, particularly Bif1B and Bif1C have been found to be more prevalent in neuronal type cells and tissues. Accordingly, in one embodiment, the method can include measuring the levels of and/or detecting the presence of Bif1B and/or Bif1C to detect, diagnose and/or predict the presence or outcome of disorders, injuries, and/or diseases that cause and/or result in neurological damage such as neuronal cell death and/or degeneration in a subject.

The present disclosure is additionally directed to methods for preventing and treating neurological damage, neuronal cell damage and/or neuronal cell death. In this aspect, the method comprises administering to a subject an effective amount of a Bif1 polypeptide, polynucleotide, recombinant virus, or a composition comprising Bif1 and/or one or more particular isoforms of Bif1 (e.g., Bif1A, Bif1B, and/or Bif1C). In an additional aspect, the present disclosure is additionally directed to methods for preventing, treating, and/or decreasing the severity of disorders, injuries, and/or diseases that result in neurological and/or neuronal cell damage or cell death, comprising administering to a subject both an effective amount of Bif1 (as defined herein) and an effective amount of Optic Atrophy 1 (Opa1). Both Bif1 and Opa1 can be in the form of a polypeptide, polynucleotide encoding the polypeptide, recombinant virus encoding the polypeptide, or a composition comprising both Bif1 and/or one or more particular isoforms of Bif1 (e.g., Bif1A, Bif1B, and/or Bif1C) and Opa1. In a more particular embodiment, the disorders, injuries, and/or diseases resulting in neurological and/or neuronal cell damage or cell death can include, as non-limiting examples, stroke, Alzheimer's disease, Parkinson's disease, traumatic brain injury, retinal degeneration, among others. The present disclosure additionally provides compositions comprising Bif1 and/or particular isoforms of Bif1 (e.g., Bif1A, Bif1B, and/or Bif1C). It is contemplated that a composition of the present disclosure can be a pharmaceutical composition. The composition can optionally comprise a pharmaceutically acceptable carrier. In another aspect, the present disclosure is directed to an isolated nucleic acid encoding a sequence of SEQ ID NO.: 1, SEQ ID NO.:2, SEQ ID NO.: 3, SEQ ID NO.:4, or SEQ ID NO.: 5. The present disclosure is additionally directed to an isolated peptide and/or peptide fragment having the peptide sequence of SEQ ID NO.: 1, SEQ ID NO.:2, SEQ ID NO.: 3, SEQ ID NO.:4, or SEQ ID NO.: 5. The present disclosure also is related to compositions and/or pharmaceutical compositions comprising SEQ ID NO.:1 and/or SEQ ID NO.: 2 and/or SEQ ID NO.: 3 and/or SEQ ID NO.:4 and/or SEQ ID NO.: 5. The present disclosure is also related to isolated nucleic acids selected from the group consisting of SEQ ID. NOs.: 6-10 or polypeptides/peptides encoded from the isolated nucleic acids selected from the group consisting of SEQ ID. NOs.: 6-10.

Another aspect provided herein relates to a method of treating a neurological disease or disorder, the method comprising: administering a therapeutically effective amount of a composition comprising a Bif-1 polypeptide or a nucleic acid encoding a Bif-1 polypeptide to a subject having a neurological disease or disorder.

In one embodiment of this aspect and all other aspects described herein, the Bif-1 polypeptide is a neuron-specific Bif-1 polypeptide.

In another embodiment of this method and all other methods and assays described herein, the neuron-specific Bif-1 polypeptide comprises Bif-1b or Bif-1c.

In another embodiment of this method and all other methods and assays described herein, the neurological disease or disorder is selected from the group consisting of: Alzheimer's disease, Parkinson's disease, dementia, multiple sclerosis, amyotrophic lateral sclerosis (ALS), blood brain barrier permeability, vascular dementia, and other neurodegenerative diseases and disorders. Each of these neurological disease or disorders involves neuronal cell stress (e.g., ischemia, hypoxia, among others) and/or neuronal cell death (e.g., apoptotic death, necrotic or death from excessive autophagy).

In another embodiment of this method and all other methods and assays described herein, the neurological disease or disorder comprises ischemic injury, hypoxic injury or apoptosis.

In another embodiment of this method and all other methods and assays described herein, the ischemic and/or hypoxic injury comprises stroke, transient ischemic attack, vasoconstriction, anastomoses, brain surgery, tumor, embolism, aneurysm, arteriosclerosis, concussion, and brain injury/trauma, among others.

In another embodiment of this method and all other methods and assays described herein, the Bif-1 polypeptide or nucleic acid encoding a Bif-1 polypeptide is administered to the brain, the spinal cord, or to a peripheral nerve ending.

In another embodiment of this method and all other methods and assays described herein, the Bif-1 polypeptide or nucleic acid encoding a Bif-1 polypeptide is administered systemically.

In another embodiment of this method and all other methods and assays described herein, the method further comprises, prior to administering said polypeptide or nucleic acid, the step of measuring the amount of Bif-1 polypeptide in a sample from said subject, wherein the Bif-1 polypeptide or nucleic acid is only administered if the measured level of Bif-1 polypeptide is reduced relative to a reference amount.

Also provided herein in another aspect are methods relating to reducing amyloid-beta-mediated neurotoxicity, the method comprising administering a therapeutically effective amount of a composition comprising a Bif-1 polypeptide or a nucleic acid encoding a Bif-1 polypeptide to a subject or to a neuronal tissue of or to a neuron of a subject with amyloid-beta mediated disease.

In another embodiment of this method and all other methods and assays described herein, the Bif-1 polypeptide is a neuron-specific Bif-1 polypeptide.

In another embodiment of this method and all other methods and assays described herein, the Bif-1 polypeptide comprises Bif-1b or Bif-1c.

In another embodiment of this method and all other methods and assays described herein, the method further comprises, prior to administering said polypeptide or nucleic acid, the step of measuring the amount of Bif-1 polypeptide in a sample from said subject, wherein the Bif-1 polypeptide or nucleic acid is only administered if the measured level of Bif-1 polypeptide is reduced relative to a reference amount.

Also provided herein are methods for predicting sensitivity of a subject to neurological damage comprising:(a) measuring the amount of a Bif-1 polypeptide or fragment thereof in a subject, and (b) comparing the amount of the Bif-1 polypeptide or fragment thereof to a reference value, wherein a decrease in the amount of the Bif-1 polypeptide or fragment thereof indicates that the subject has an increased sensitivity to neurological damage.

In another embodiment of this method and all other methods and assays described herein, the neurological damage comprises ischemic injury or hypoxic injury.

In another embodiment of this method and all other methods and assays described herein, the neurological damage comprises neuronal cell death.

In another embodiment of this method and all other methods and assays described herein, the neurological damage comprises apoptosis.

In another embodiment of this method and all other methods and assays described herein, the amount of Bif-1 polypeptide or fragment thereof is measured in a biological sample obtained from the subject.

In another embodiment of this method and all other methods and assays described herein, the biological sample comprises cerebral spinal fluid, urine, saliva, blood, plasma, biopsy sample or a tumor sample.

In another embodiment of this method and all other methods and assays described herein, the biological sample comprises cerebral spinal fluid.

In another embodiment of this method and all other methods and assays described herein, the Bif-1 polypeptide comprises Bif-1b or Bif-1c.

In another embodiment of this method and all other methods and assays described herein, the Bif-1 polypeptide comprises a neuron-specific Bif-1 polypeptide.

In another embodiment of this method and all other methods and assays described herein, the Bif-1 polypeptide or fragment thereof is detected in vivo.

In another embodiment of this method and all other methods and assays described herein, the method further comprises administering a Bif-1 polypeptide or nucleic acid encoding a Bif-1 polypeptide if the measured Bif-1 polypeptide or fragment thereof is reduced relative to the reference value.

In another embodiment of this method and all other methods and assays described herein, the Bif-1 polypeptide or fragment thereof is detected using magnetic resonance imaging (MRI), positron emission tomography (PET), CT scan, or nuclear magnetic resonance imaging (NMR), among others.

Also provided herein are in vivo assays comprising: (a) administering an agent that binds to a Bif-1 polypeptide (or a fragment thereof) or an mRNA encoding a Bif-1 polypeptide (or a fragment thereof) to a subject, (b) detecting the amount of the agent bound to the Bif-1 polypeptide or fragment thereof or the Bif-1 mRNA or fragment thereof and determining the amount of Bif-1 polypeptide, and (c) comparing the amount of the Bif-1 polypeptide or Bif-1 mRNA to a reference value, wherein a decrease in the amount of Bif-1 compared to the reference value indicates that the subject has an increased risk of neurological damage.

In another embodiment of this assay and all other methods and assays described herein, the agent comprises a detectable moiety.

In another embodiment of this assay and all other methods and assays described herein, the Bif-1 polypeptide comprises Bif-1b or Bif-1c.

In another embodiment of this assay and all other methods and assays described herein, the Bif-1 polypeptide comprises a neuron-specific Bif-1 polypeptide.

In another embodiment of this assay and all other methods and assays described herein, the bound agent is detected using magnetic resonance imaging (MRI), positron emission tomography (PET), CT scan, and nuclear magnetic resonance imaging (NMR), among others.

In another embodiment of this assay and all other methods and assays described herein, a Bif-1 polypeptide or nucleic acid encoding a Bif-1 polypeptide is administered to the subject if the measured Bif-1 polypeptide or fragment thereof is reduced relative to the reference value

Also provided herein are in vitro assays comprising: (a) contacting a biological sample obtained from a subject with an agent that binds to a Bif-1 polypeptide, a Bif-1 polypeptide fragment, an mRNA encoding a Bif-1 polypeptide, or a fragment thereof, (b) detecting the amount of the agent bound to the Bif-1 polypeptide, the Bif-1 polypeptide fragment, the mRNA encoding the Bif-1 polypeptide or fragment thereof, and (c) comparing the amount of the Bif-1 polypeptide or Bif-1 mRNA to a reference value, wherein a decrease in the amount of Bif-1 compared to the reference value indicates that the subject has an increased risk of neurological damage.

In another embodiment of this assay and all other methods and assays described herein, the biological sample comprises cerebral spinal fluid, urine, saliva, blood, plasma, biopsy sample or a tumor sample.

In another embodiment of this assay and all other methods and assays described herein, the biological sample comprises cerebral spinal fluid.

In another embodiment of this assay and all other methods and assays described herein, the Bif-1 polypeptide comprises Bif-1b or Bif-1c.

In another embodiment of this assay and all other methods and assays described herein, the Bif-1 polypeptide comprises a neuron-specific Bif-1 polypeptide.

In another embodiment of this assay and all other methods and assays described herein, the agent comprises a detectable moiety.

Also provided herein are in vitro assays comprising:(a) detecting a Bif-1 polypeptide or fragment thereof in a biological sample obtained from a subject using mass spectrometry, (b) comparing the mass spectrum of step (a) with the mass spectrum of a Bif-1 recombinant protein standard, wherein a decrease in the amount of Bif-1 polypeptide or fragment thereof compared to the protein standard indicates that the subject has an increased risk of neurological damage.

In one embodiment of this assay and all other methods and assays described herein, the neurological damage comprises reduced cognition, reduced learning and memory, increased seizures or reduced longevity.

In another embodiment of this assay and all other methods and assays described herein, the assay further comprises a step of contacting the Bif-1 recombinant protein standard and/or the Bif-1 polypeptide or fragment thereof with a protease.

In another embodiment of this assay and all other methods and assays described herein, the protease is trypsin or pepsin.

Another aspect provided herein relates to a method of treating a neurological disease or disorder, the method comprising: administering a therapeutically effective amount of a composition comprising an inhibitor of Bif-1 expression and/or activity to a subject having a neurological disease or disorder.

In one embodiment of this method and all other methods and assays described herein, the subject has been found to have increased Bif-1 expression and/or activity in a neuronal tissue relative to a healthy reference level.

In another embodiment of this method and all other methods and assays described herein, the neurological disease or disorder is impaired cognitive function, learning or memory.

In another embodiment of this method and all other methods and assays described herein, the neurological disease comprises abnormally increased autophagy.

In another embodiment of this method and all other methods and assays described herein, wherein the neurological disease comprises Parkinson's disease.

In another embodiment of this method and all other methods and assays described herein, the neurological disease comprises reduced apoptosis (e.g., certain developmental disorders or cancer involving stem cells or neural progenitor cells or glial progenitor cells).

In another embodiment of this method and all other methods and assays described herein, the neurological disease comprises a cancer. Without wishing to be bound by theory, it is likely in cancer that there is not enough Bif-1 to promote apoptosis or elevated Bif-1 may enhance autophagy, thereby enhancing survival of tumor cells subject to stress. There are reports that Bif-1 levels are both increased and decreased in malignant tumor samples. It is contemplated that shifting the balance of Bif-1 polypeptide or neuron-specific isoforms thereof towards that found in healthy, non-tumor tissue can be useful in tumor therapy.

Also provided herein are uses of a composition comprising a Bif-1 polypeptide or a nucleic acid encoding a Bif-1 polypeptide for treatment of a subject having a neurological disease or disorder. In addition, uses of a composition a Bif-1 polypeptide or a nucleic acid encoding a Bif-1 polypeptide for treatment of a subject having amyloid-beta-mediated neurotoxicity are also contemplated herein.

### BRIEF DESCRIPTION OF THE FIGURES

FIGS. 1A-1B show that Bif-1 is pro-survival in neurons. FIG. 1A: Loss of Bif-1 enhanced neuronal death caused by camptothecin (CPT; 5 μM, 12 hr). Bar=25 μm. *: p<0.001 vs. Bif-1 shRNA/DMSO, two-way ANOVA with Tukey post hoc test. FIG. 1B: Western blot analysis of activated caspase-3 expression confirmed that Bif-1 shRNA infected neurons display increased sensitivity to CPT-induced apoptosis. Data are expressed as fold-elevation of activated caspase-3/actin relative to control (DMSO)/control shRNA. *: p<0.05 vs. DMSO/control shRNA. #: p<0.05 vs. CPT/control shRNA. $: p<0.05 vs. DMSO/Bif-1 shRNA, two-way ANOVA with Tukey post hoc test. All images and blots are representative of 3 separate experiments. Bars represent mean±SEM.

FIGS. 2A-2E show data indicating that loss of Bif-1 in neurons results in fragmented mitochondria. FIG. 2A: Mitochondria in primary postnatal cortical neurons from Bif-1−/− mice, as visualized with MitoDsRed2 fluorescence, are noticeably smaller and more punctate compared to those in Bif-1+/+ neurons. Unlike Bif-1+/+ neurons, they contained more mitochondria with a perinuclear distribution rather than neuritic. These changes in size and distribution of mitochondria in Bif-1−/− neurons were partially corrected by overexpression of Bif-1 isoforms, with Bif-1c being most effective. Upon overexpression, Bif-1c localization was more punctate, in contrast with the more uniformly cytosolic distribution of Bif-1a and Bif-1b. Nuclei are visualized with Hoechst 33258 dye. FIG. 2B: Similar to Bif-1−/− neurons, wild-type neurons infected with Bif-1 shRNA contained fragmented, punctate mitochondria that were absent from neurites. Neurons infected with Mfn2 shRNA as a positive control for induced mitochondrial fission contained similarly fragmented mitochondria indicative of mitochondrial fission, as previously reported (22). FIG. 2C: Knockout or knockdown of Bif-1 resulted in shorter neuritic mitochondria by electron microscopy analysis. Bif-1 deficient neurons also displayed a significant reduction in the total number of mitochondria per area.*: p<0.05, **: p<0.01, Student's t-test (n=15-20 for mitochondrial length, n=10 for number of mitochondria per neurite area). FIG. 2D: Bif-1 knockdown resulted in mitochondrial membrane depolarization, as revealed by a red-to-green shift of JC-1 fluorescence. EGFP fluorescence, used as an infection marker of the shRNA, was very weak relative to JC-1 green fluorescence and was therefore judged as negligible. The data are represented as the percentage of red fluorescence intensity compared to total fluorescence intensity (red+green). Bars represent mean±SEM. *: p<0.05, Student's t-test (n=3) FIG. 2E: Mouse embryonic fibroblasts infected with Bif-1 shRNA contained elongated and more interconnected mitochondria, while those infected with Mfn2 shRNA displayed smaller, punctate mitochondria. Bar in FIGS. 2A, 2B, 2E=10 μm. Bar in C=500 nm Bar in FIG. 2D=20 μm. All images are representative of 2-3 separate experiments.

FIGS. 3A-3C show Bif-1 expression in neuronal and non-neuronal cells. FIG. 3A: Primary neurons and neuronal cell lines contain two specific alternatively spliced mRNA isoforms, Bif-1b and Bif-1c, not expressed in other cell types. Expression levels of Bif-1 mRNA were analyzed by RT-PCR using primers flanking exons 6 and 7, the sites of alternative Bif-1 splicing. FIG. 3B: Neurons predominantly express the neuron-specific isoforms of Bif-1 protein. Expression of Bif-1 protein was analyzed by western blot using a non-isoform-specific antibody. Neuron-specific isoforms (Bif-1b/c) are not separable under the conditions used. h-SH-SY5Y, human neuroblastoma cell line; m-PNC neurons, mouse postnatal cortical neurons; m-NPC, mouse neural progenitor cells derived from spinal cord; FGF, fibroblast growth factor; m-Neuro-2A, mouse neuroblastoma cell line; m-NIH3T3, mouse fibroblast cell line; m-MEF, mouse embryonic fibroblasts. FIG. 3C: Schematic showing the exon structures of the various Bif-1 isoforms. All splice sites are located within the N-BAR domain, which is responsible for Bif-1 membrane binding function. Neuron-specific isoforms are shown. *, see text for Bif-1e.

FIGS. 4A-4B show data indicating that Bif-1 expression is decreased in the penumbra after focal ischemia. FIG. 4A: Mito-CFP mice (Bif-1 wild-type) were subjected to middle cerebral artery occlusion (MCAO) for 45 minutes. After 2 days, tissue dorsal to the infarct (visualized by TTC in adjacent slices) was analyzed for protein expression. The corresponding region of tissue from the contralateral side was taken as a control. Data from 4 different animals are shown with some irrelevant lanes cut off in the middle of the blot. FIG. 4B: Densitometric quantification of the western blots. Both Bif-1b/c and Bif-1a expression were decreased in the ischemic penumbra, with Bif-1b/c more greatly affected. Neither Tuj1 (neuronal specific marker) nor neuron specific Mito-CFP expression were decreased, indicating the loss in Bif-1 expression was not due to loss of neurons. Bars represent mean±SEM. *: p<0.05, **: p<0.01, Student's t-test (n=4).

FIGS. 5A-5B show data indicating that Bif-1−/− mice have increased sensitivity to ischemic injury in the MCAO model. FIG. 5A: Bif-1−/− animals had larger infarct volumes after MCAO injury (45 min occlusion, 2 day reperfusion) than Bif-1+/+ controls, using serial coronal slices stained with TTC. Bars represent mean±SEM. *: p<0.05, Student's t-test (N=10). FIG. 5B: Neuronal mitochondria in the ischemic penumbra in the cortex (right panels) are more fragmented in Bif1−/− animals relative to Bif-1+/+ animals. Also, control mitochondrial morphology (contralateral, left panels) was also more fragmented and the overall Mito-CFP signal was weaker in Bif1−/− animals. Bar in B=10 μm. Images are representative of 4 animals.

FIG. 6 shows data indicating that overexpression of Bif-1 has no effect on caspase-3 activation induced by camptothecin (CPT) in neurons. Neurons overexpressing various Bif-1 isoforms showed similar base (DMSO) and CPT-induced levels of activated caspase-3 compared to control virus-infected neurons. Bif-1 isoform overexpression also had no effect on CPT-induced neuron death (not shown), based on morphological criteria previously reported (14, 20). Blots are representative of 3 separate experiments.

FIGS. 7A-7B show data indicating that Bif-1 is pro-apoptotic in fibroblasts. FIG. 7A: In contrast to neurons, elevated Bif-1 expression sensitized mouse embryonic fibroblasts to CPT-induced apoptosis. FIG. 7B: Bif-1a overexpression also sensitized NIH3T3 fibroblasts to CPT-induced apoptosis. Blots are representative of 2-3 separate experiments.

FIG. 8 shows a time-course experiment depicting the effects of Bif-1 knockdown on mitochondrial morphology and localization in cultured primary cortical neurons. Neurons were infected for either 48 or 72 hours with control shRNA or Bif-1 shRNA lentiviruses prior to fixation. After 48 hours, mitochondria (MitoDsRed2 stained) in neurites begin to fragment and disappear from neurites. Arrowheads denote areas undergoing mitochondrial fragmentation. After 72 hours, mitochondria are almost completely absent from neurites and begin to fragment in the soma as well. Bif-1 immunoreactivity is stained using a green dye. Nuclei are visualized with Hoechst 33258 dye. Bar=10 μm.

FIGS. 9A-9B show that modulation of Bif-1 levels does not alter expression of key proteins involved in mitochondrial dynamics in neurons. FIG. 9A Bif-1 overexpression did not change the levels of the mitochondrial fission protein Drp1 or the fusion protein Mfn2. FIG. 9B Bif-1 knockdown did not affect the levels of Drp1 or Mfn2. Blots are representative of 3 separate experiments.

FIGS. 10A-10F show data indicating that Bif1−/− animals have an exaggerated astroglial response after MCAO injury. FIGS. 10A-10D: Two days after MCAO injury, astrocytes around the infarct are pushed further towards the medial septum in Bif1−/− animals, corresponding to a larger infarct volume. Basal levels and distribution of GFAP expression on the unaffected contralateral side were similar in Bif1−/− and wild-type animals. FIGS. 10E-10F: Astrocytes around the infarct in Bif1−/− animals were larger with longer ramified processes, a morphology typical of reactive astrocytes. Bar in A-D=200 μm. Bar in E-F=20 μm. Images are representative of results obtained from 8 animals. V, ventricle; M. Sep, medial septum.

FIG. 11 is a graph depicting the number and size of mitochondria in Bif-1 knockout mice compared to wildtype mice.

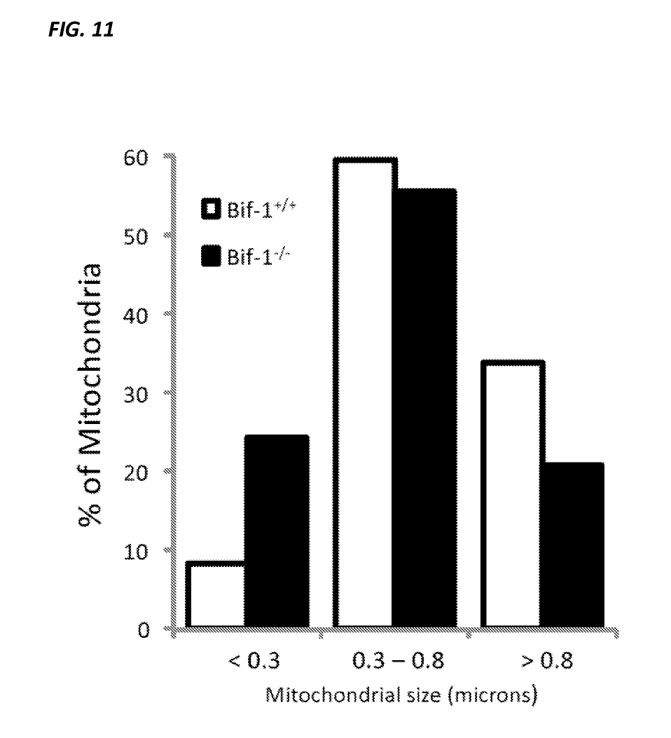

FIG. 12 is a micrograph depicting data that show Opa1 knockdown promotes decreased Bif-1 expression before caspase-3 activation. DNA damage does not reduce Bif-1 expression and Bif-1 overexpression does not protect against DNA damage by itself. However, when Opa1 is knocked down before DNA damage, Bif-1 levels decline.

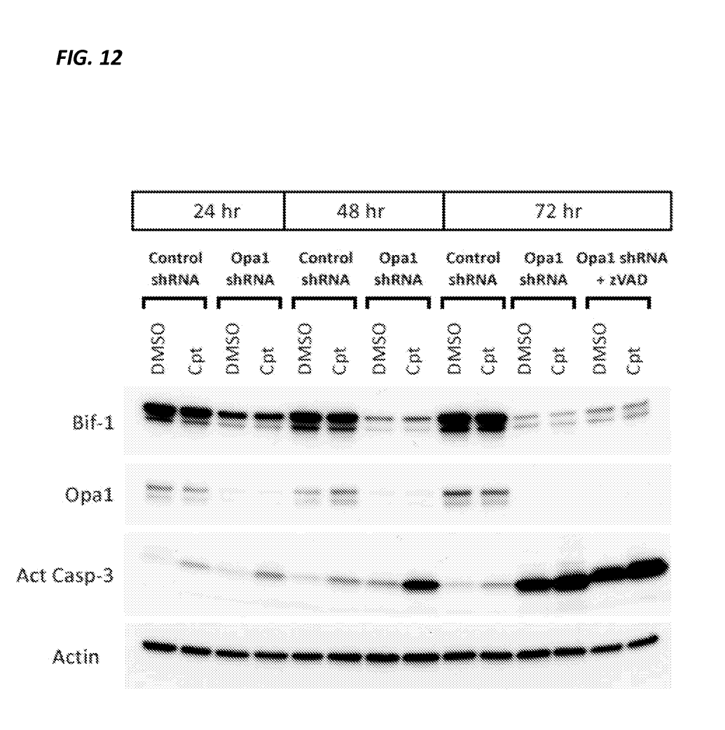

FIG. 13 is a micrograph showing that Bif-1expression mitigates cell death induced by Opa-1 depletion based on the reduction in caspase-3 activation (compare Opa1 shRNA/Cpt vs. Opa1 shRNA/Bif-1c/Cpt).

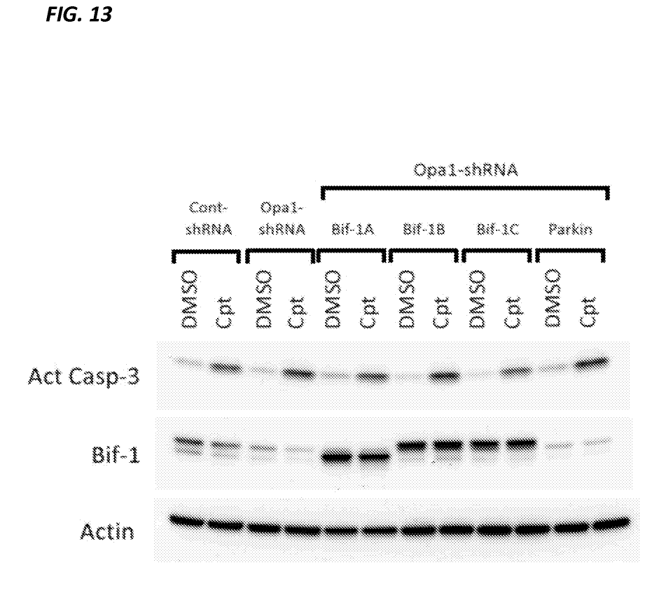

FIGS. 14A-14F are a series of micrographs showing that Bif-1c expression mitigates cell death induced by Opa-1 depletion and DNA damage (Cpt treatment). Note the fewer numbers of rounded up phase bright cells in the Bif-1c expressing cultures.

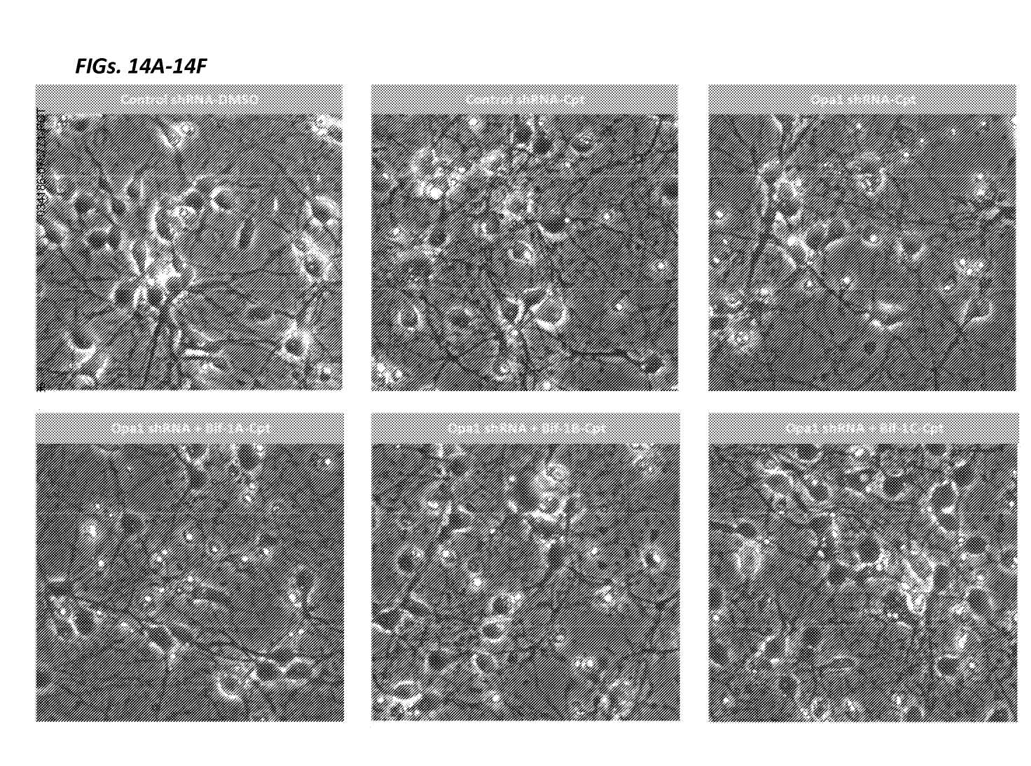

FIG. 15 is a micrograph showing that Bif-1c reduces p53 induction in response to DNA damage following Opa1 knockdown (*). Note that the p53 protein is upregulated in response to the DNA damage inducing drug camptothecin (Cpt) relative to DMSO, the control solvent. Without wishing to be bound by theory, Bif-1c could promote neuroprotection, in part, by suppressing p53 induction.

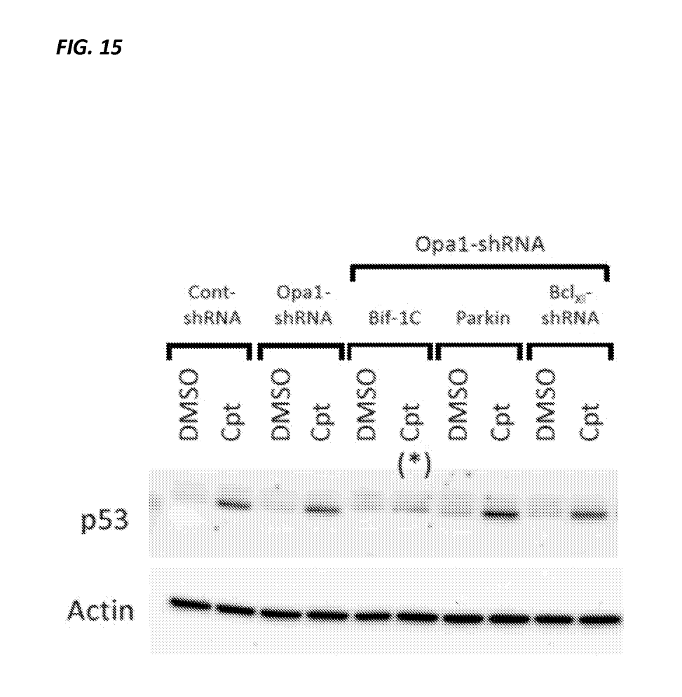

FIG. 16 is a micrograph showing another example wherein Bif-1c reduces p53 induction in response to DNA damage following Opa1 knockdown. Bif-1A and Bif-1B do not reduce p53 induction although Bif-1B does cause a shift in the migration of p53 as seen with Bif-1C. As we observed for Bif-1A/B/C with Abeta toxicity, Bif-1c is more neuroprotective than Bif-1A and Bif-1B.

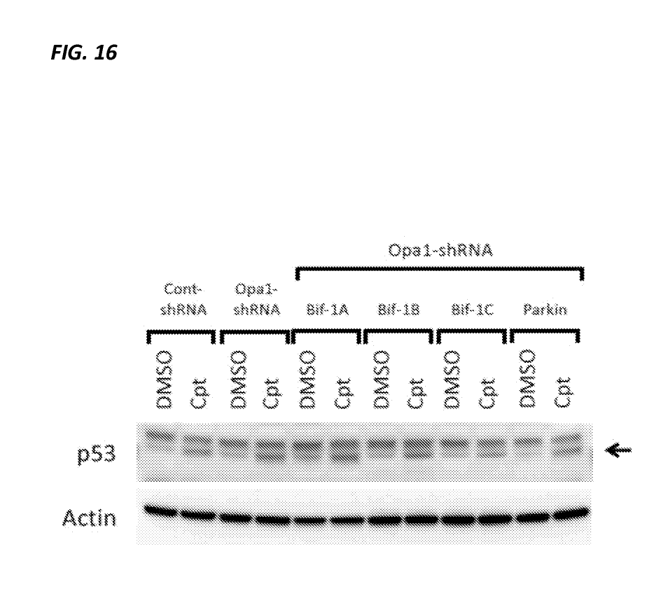

FIG. 17 is a micrograph showing data relating to Bif-1 expression in a mouse infarct model. In the middle cerebral artery occlusion model, the stroke side is the ipsilateral side. One can see much less Bif-1B/C expression in the infarct area and in some of the penumbra (sup/inf to infarct) adjacent to the infarct on the ipsilateral side compared to the opposite, unaffected side (contralateral) of the brain from the same animal. The stroke impaired side also shows an increase in the Bif-1A band. Representative animal #2 shows a reduction in Bif-1B/C in all stroke related areas compared to the contralateral side. Superior area represents the cortex, the infarct is cortex, and some stratium whereas the inferior part is striatum.

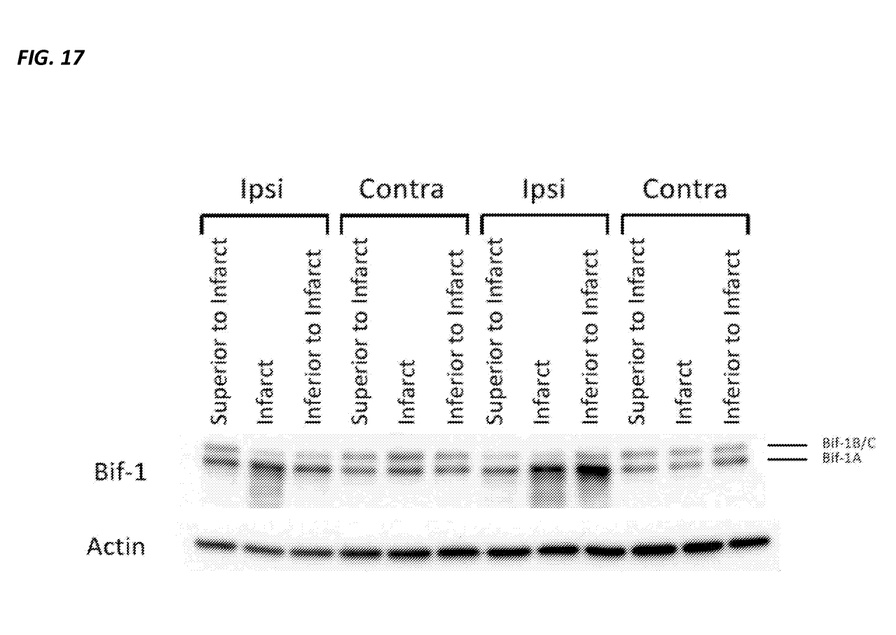

FIGS. 18A-18B show neuron specific Bif-1 isoforms. FIG. 18A shows splicing of a novel Bif-1 transcript in Neuro 2A cells runs from exon 5 directly to exon 7. FIG. 18B is the sequence for the novel splice variant detected in mouse Neuro2A cells.

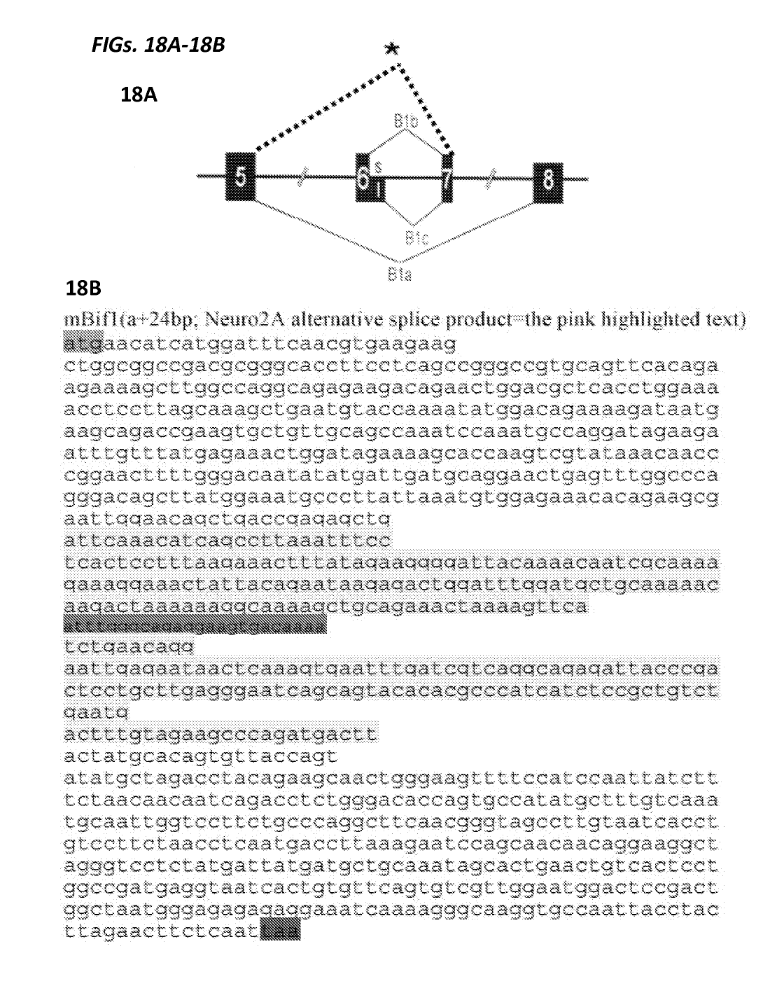

FIGS. 19A-19B are micrographs depicting expression of neuron-specific Bif-1 isoforms in different cell types from the brain.

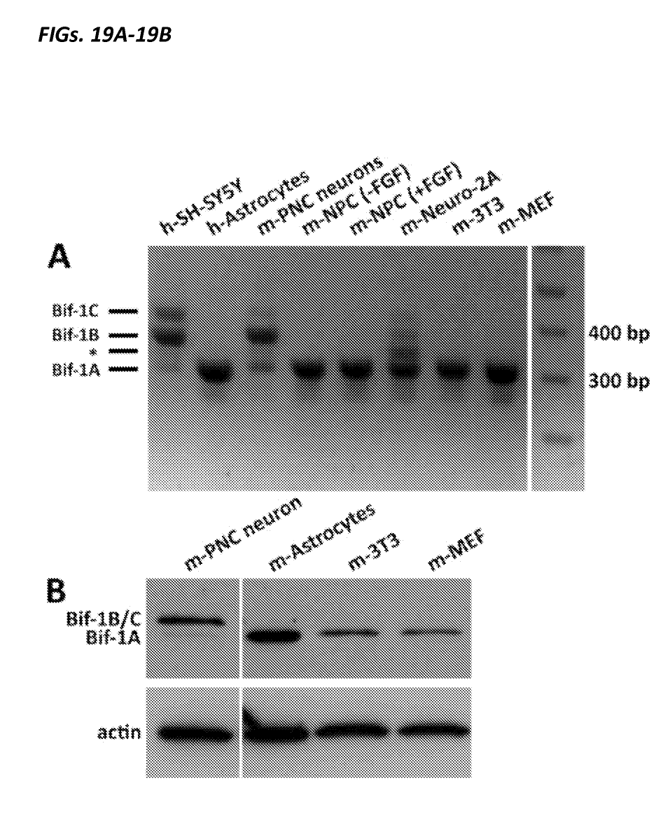

FIGS. 20A-20C are micrographs and bar graphs showing that Bif-1B/C protein is lost in brain tissue from patients with Alzheimer's Disease.

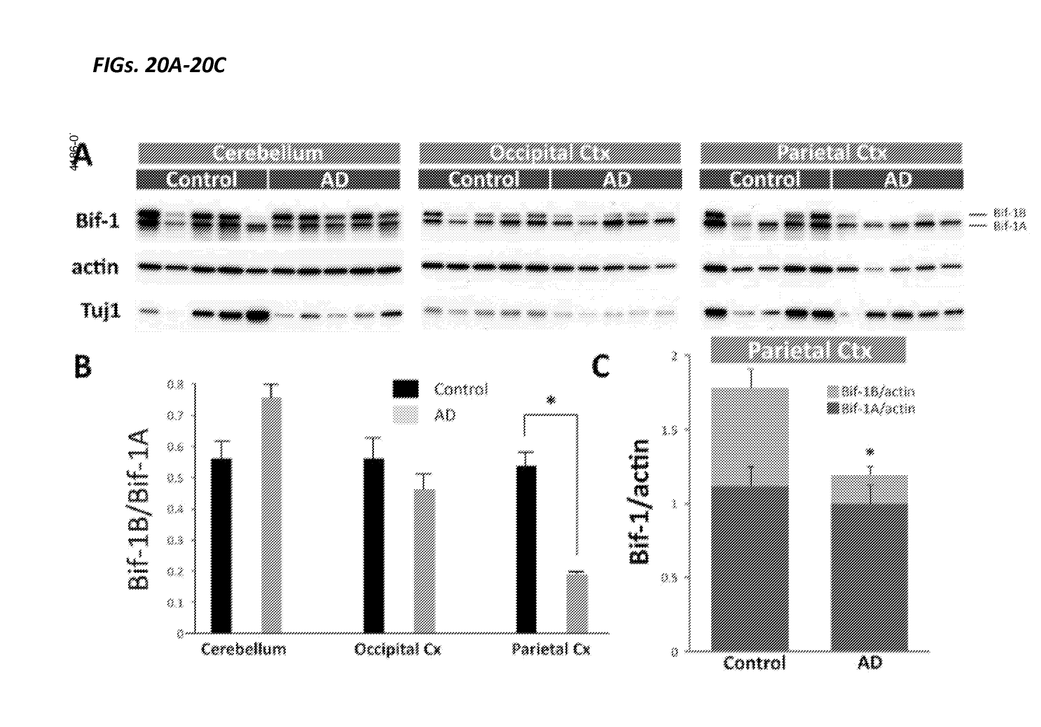

FIGS. 21A-21D are micrographs showing the role of Bif-1 in Alzheimer's Disease. FIG. 21A shows that Abeta peptide reduces Bif-1B/C expression. FIGS. 21B-21C shows that Bif-1 depletion enhances Abeta toxicity, while FIG. 21D shows that Bif-1c expression reduces cell death (caspase 3) induced by expression of mutant APP.

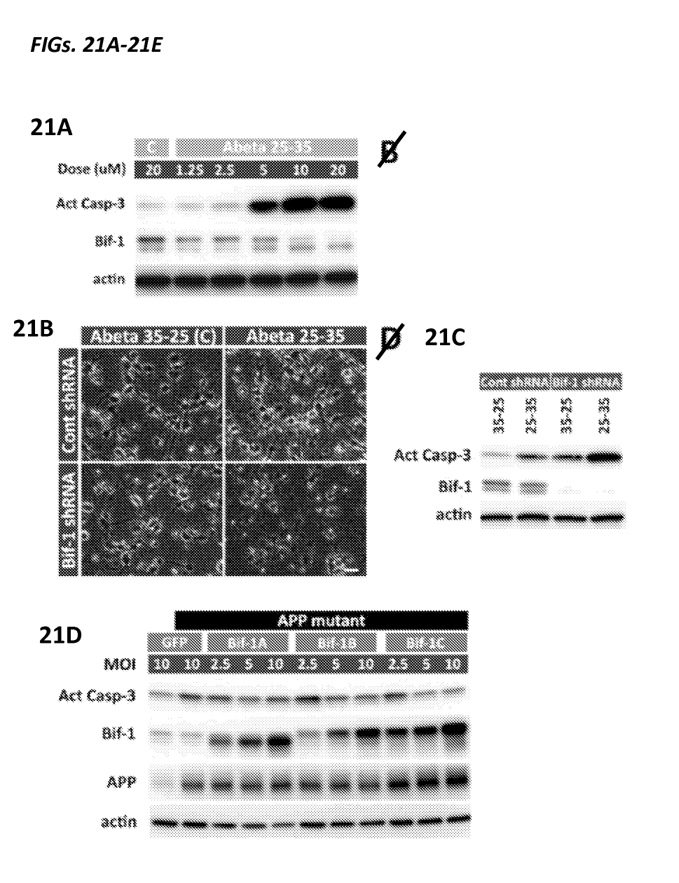

FIG. 22 is a graph showing that neuron-specific Bif-1 is lost in Alzheimer's disease patients.

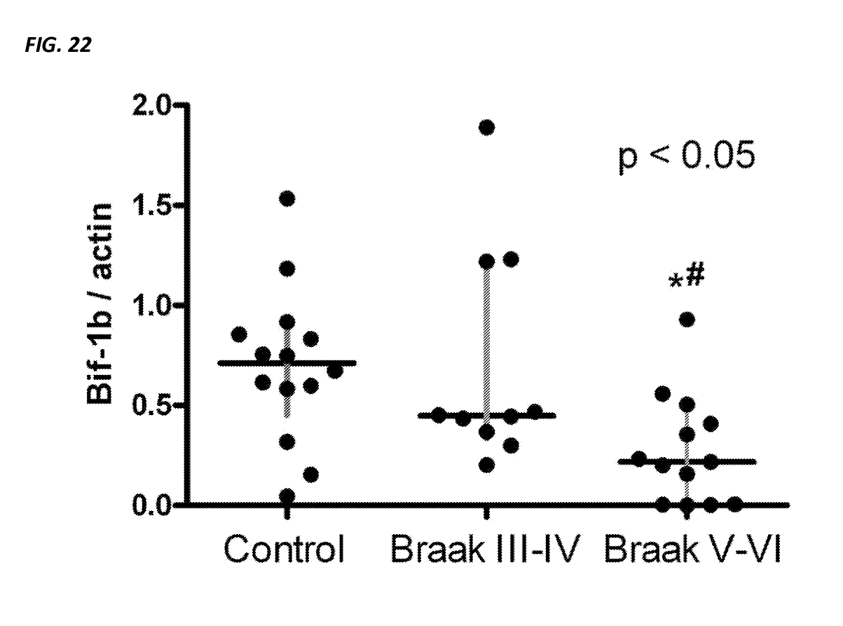

FIGS. 23A-23B are graphs showing that loss of Bif-1b does not correlate with loss of MAP2+ neurons (stereological counts) or loss of Tuj1 (neuritic marker).

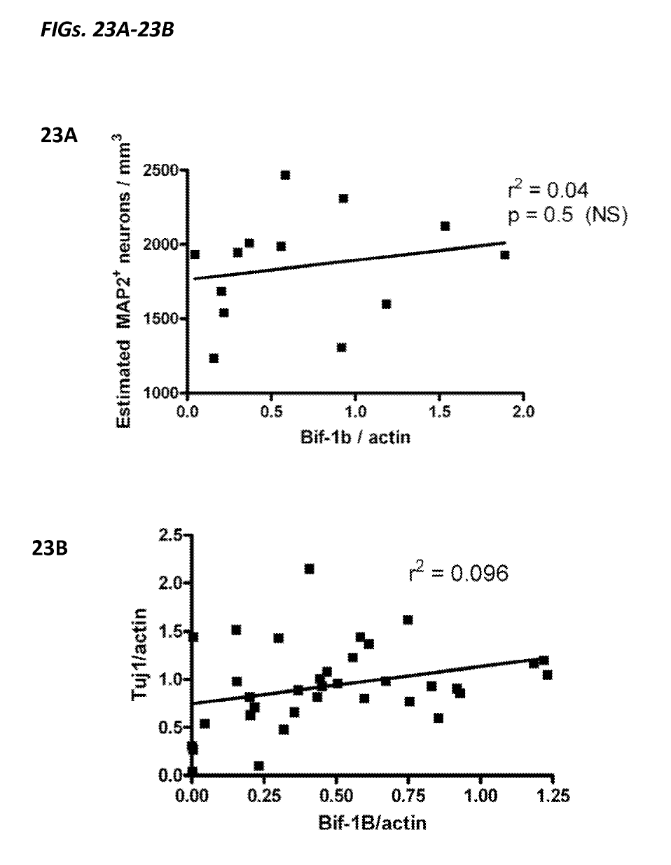

FIG. 24 is a graph showing that Bif-1b expression is decreased in synaptosomes of Alzheimer's disease patients.

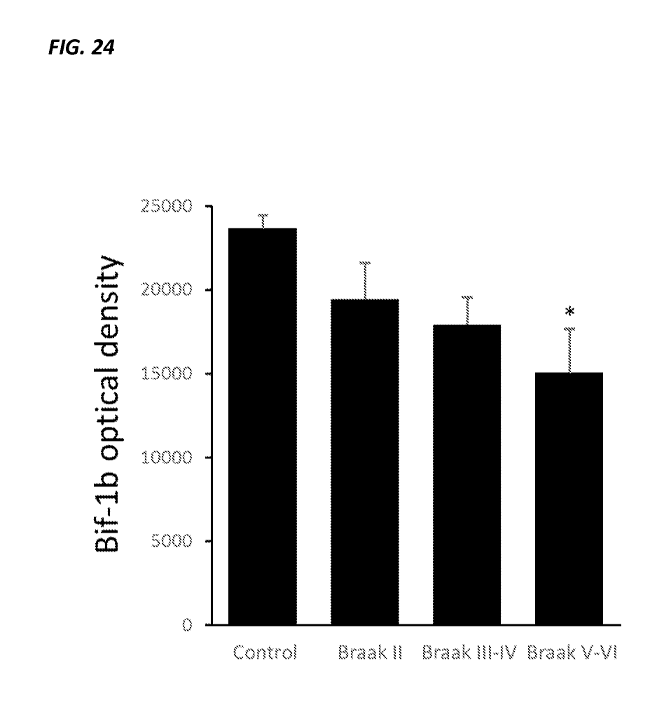

FIGS. 25A-25B are bar graphs showing that a decrease in neuron-specific Bif-1 is recapitulated in symptomatic APP/PS1 mice.

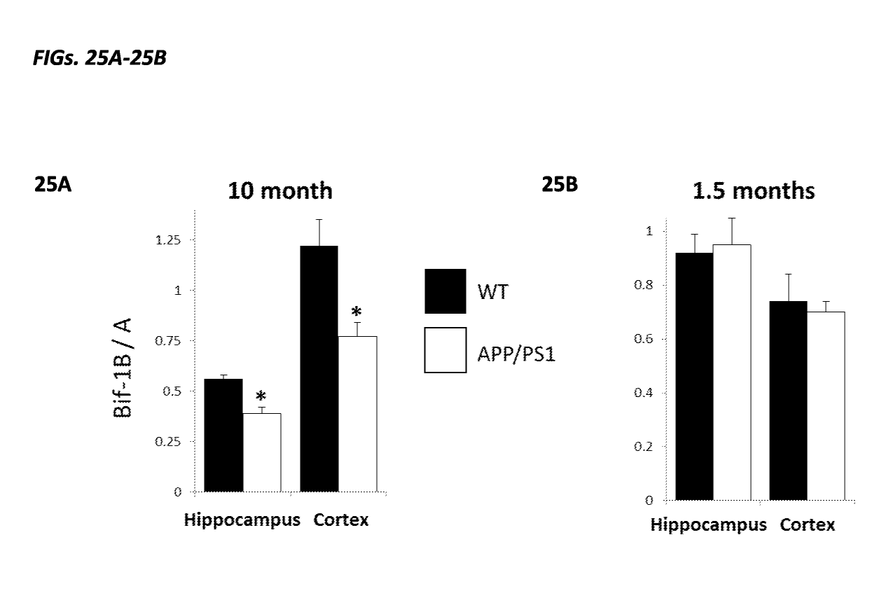

FIG. 26 is a graph showing that the absence of Bif-1 exacerbates Alzheimer's disease +/− related mortality.

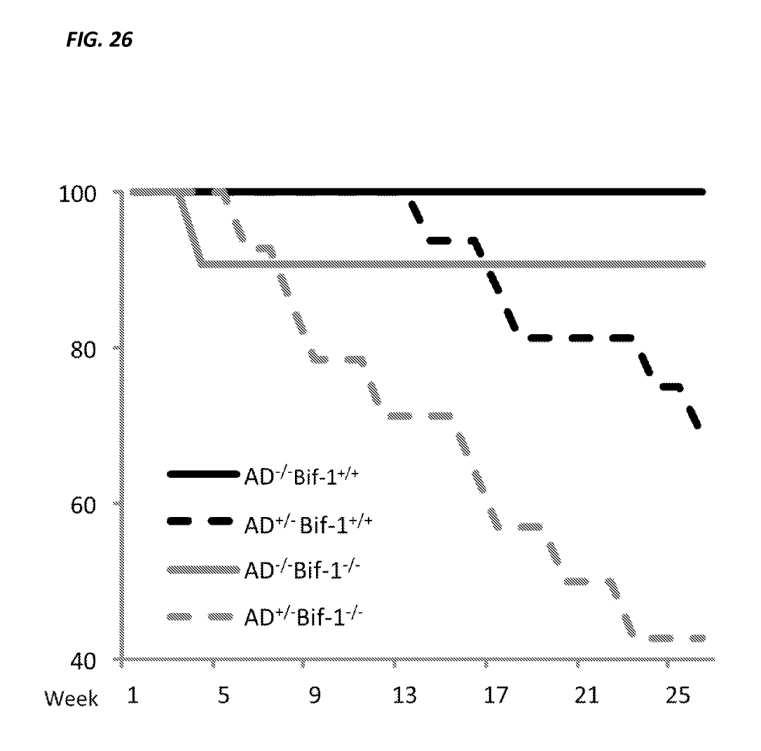

FIG. 27 is a graph showing an acquisition curve at 6 months, showing a modulated response of AD+/− Bif-1−/− compared to other groups.

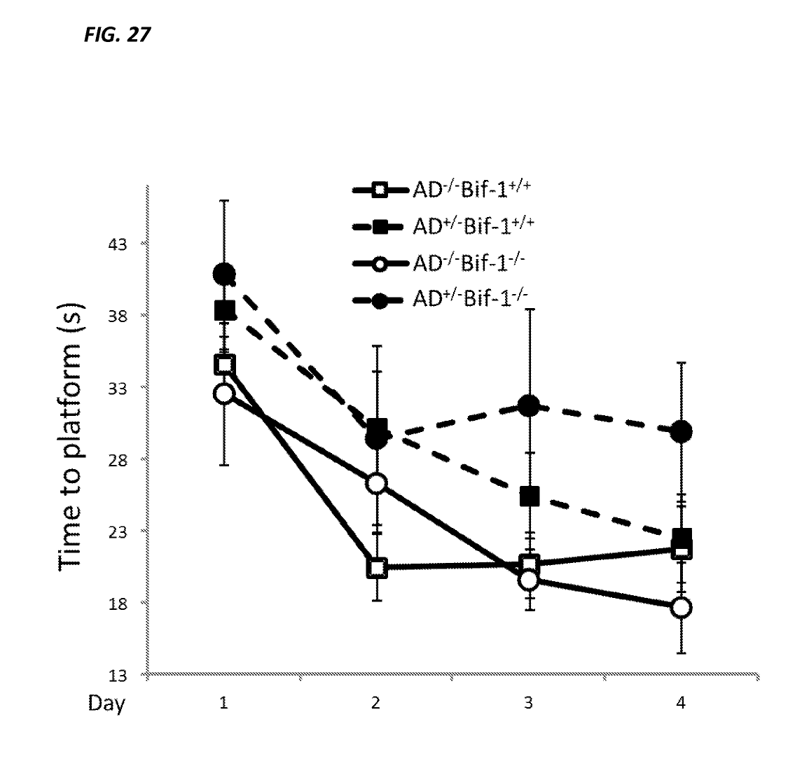

FIG. 28 is a bar graph showing that AD+/− Bif-1−/− animals spend less time in correct zone during retention.

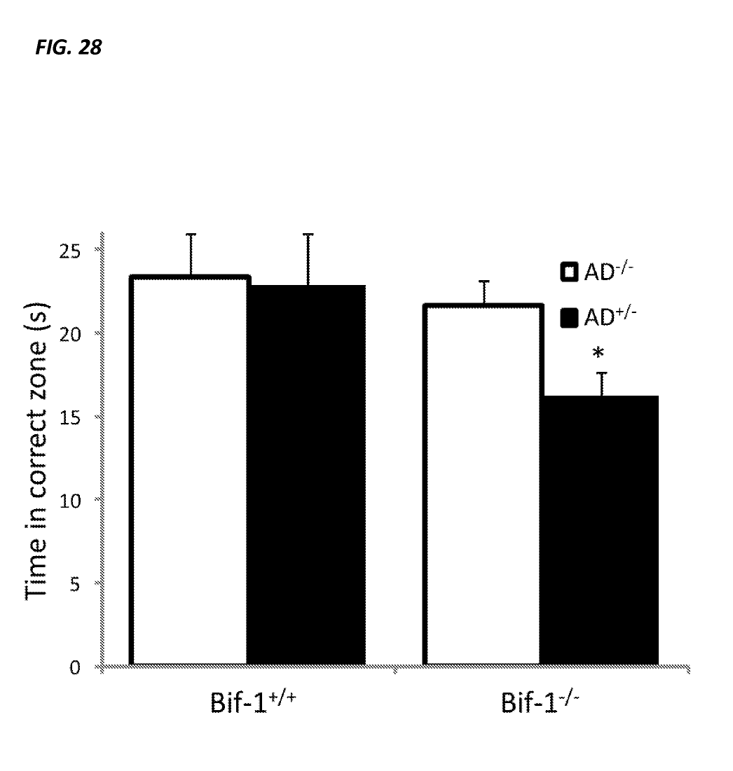

FIG. 29 is a bar graph showing that there is no difference in cumulative distance to platform during retention.

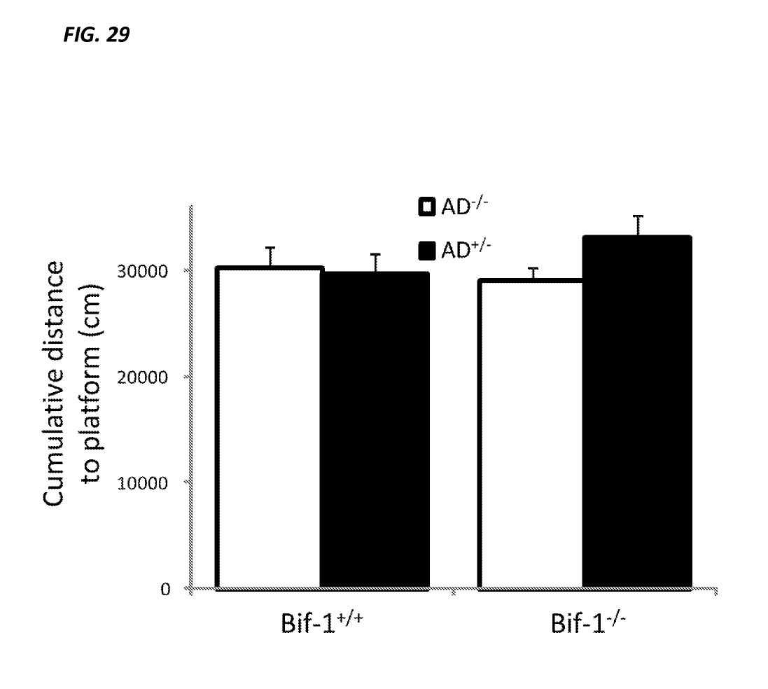

FIG. 30 is a graph showing that at 12 months, Bif-1−/− animals learn tasks slower.

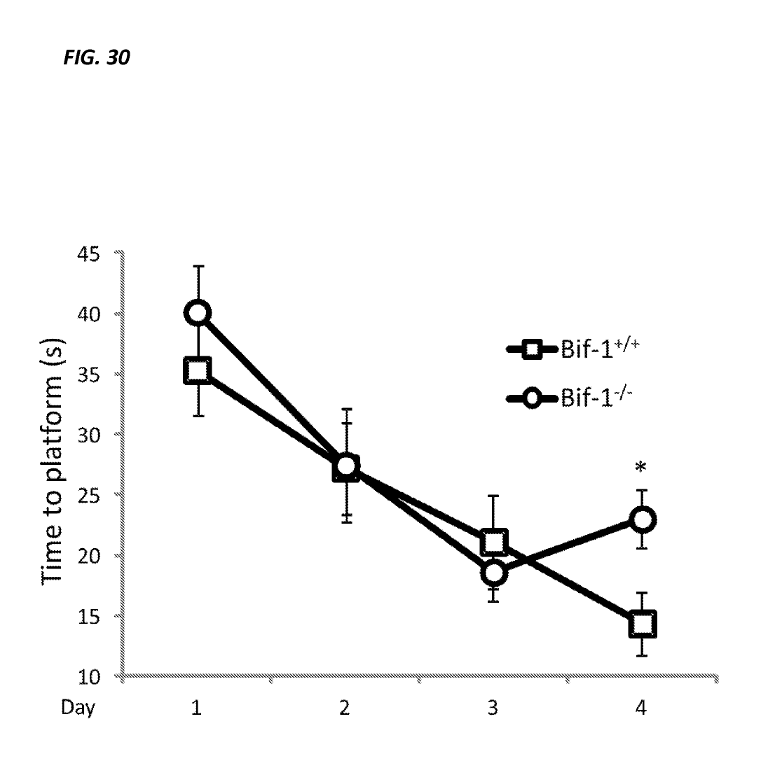

FIG. 31 is a bar graph showing that Bif-1−/− animals are farther away from the platform during retention.

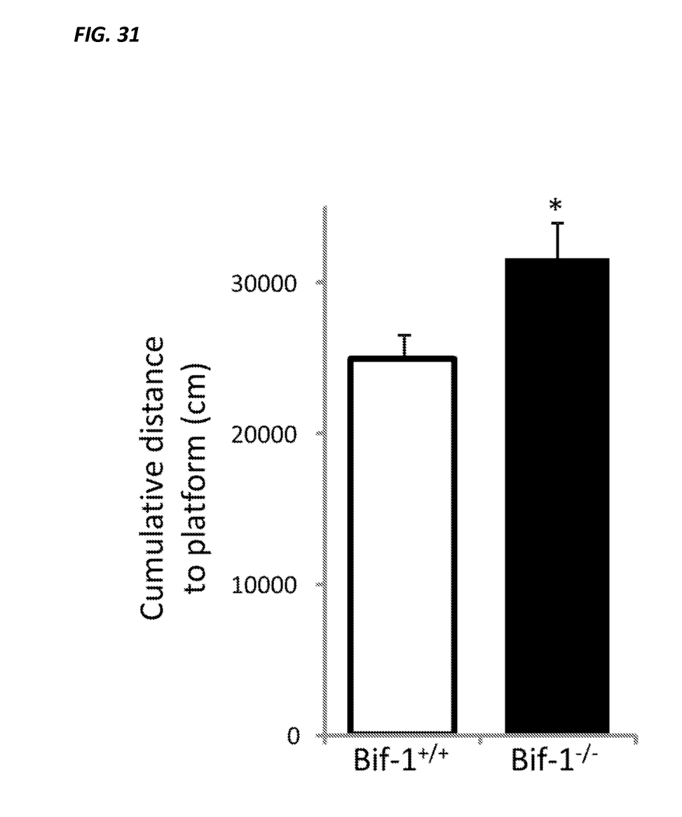

FIG. 32 is a bar graph showing that there is no difference in time in correct zone during retention in Bif-1−/− mice compared to Bif-1 +/+ mice.

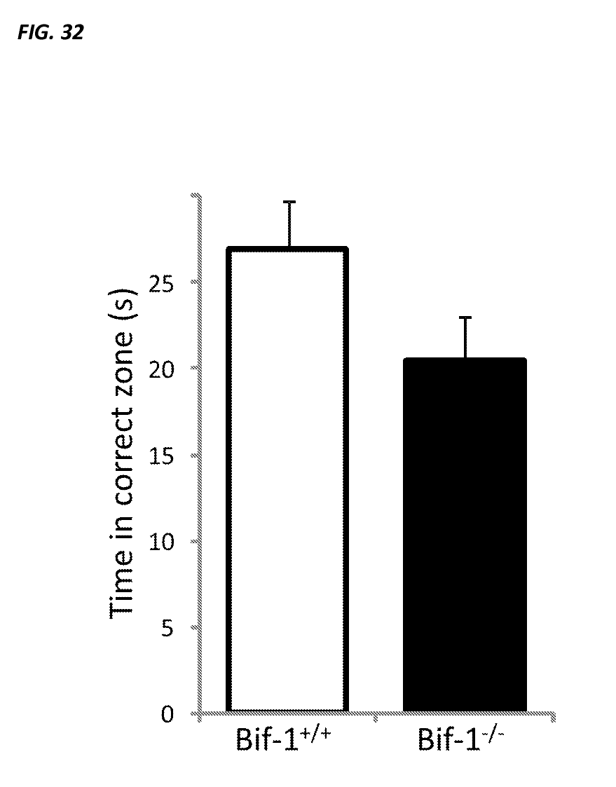

FIG. 33 is a graph showing that Bif-1−/− animals are not impaired on the rotarod.

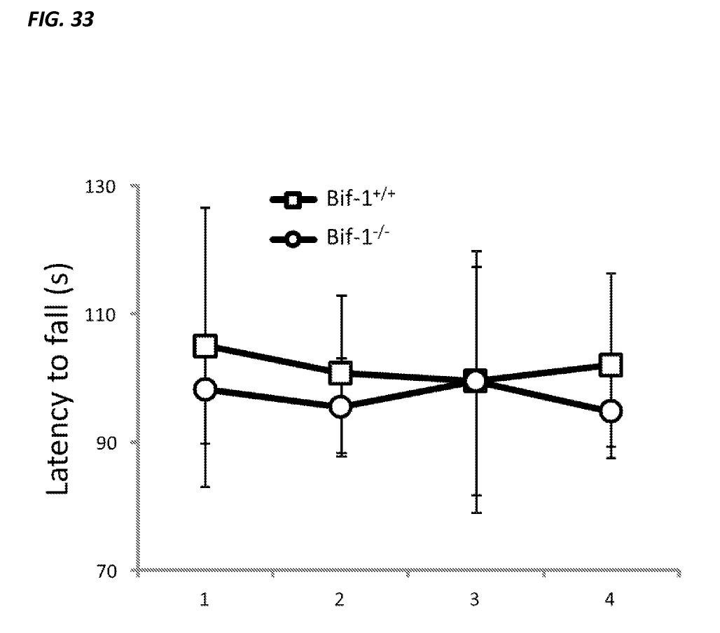

FIG. 34 is a graph showing that Bif-1−/− animals have normal grip strength.

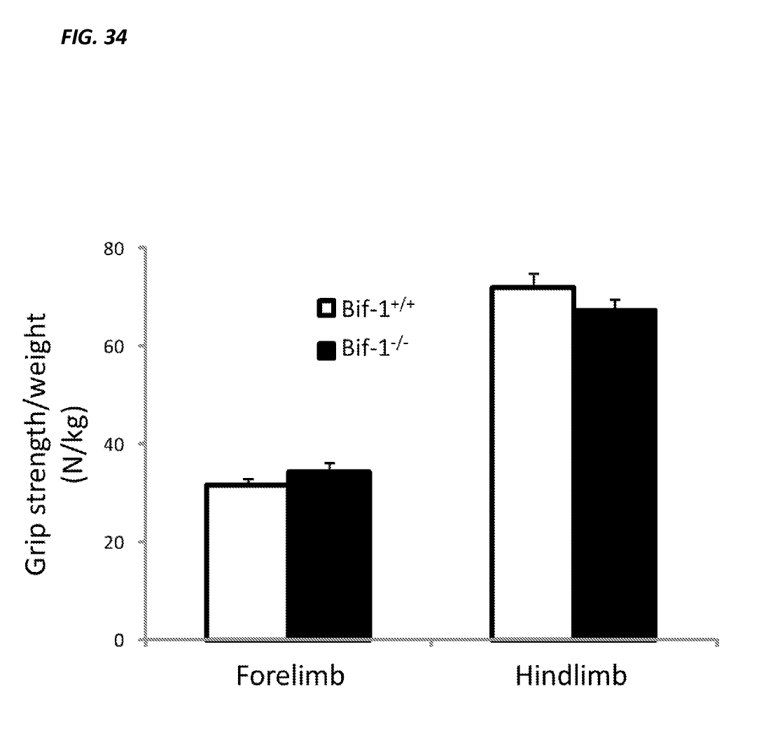

FIGS. 35A-35C are graphs showing that loss of Bif-1 enhances the presence and size of amyloid plaques, a hallmark of Alzheimer's disease pathology.

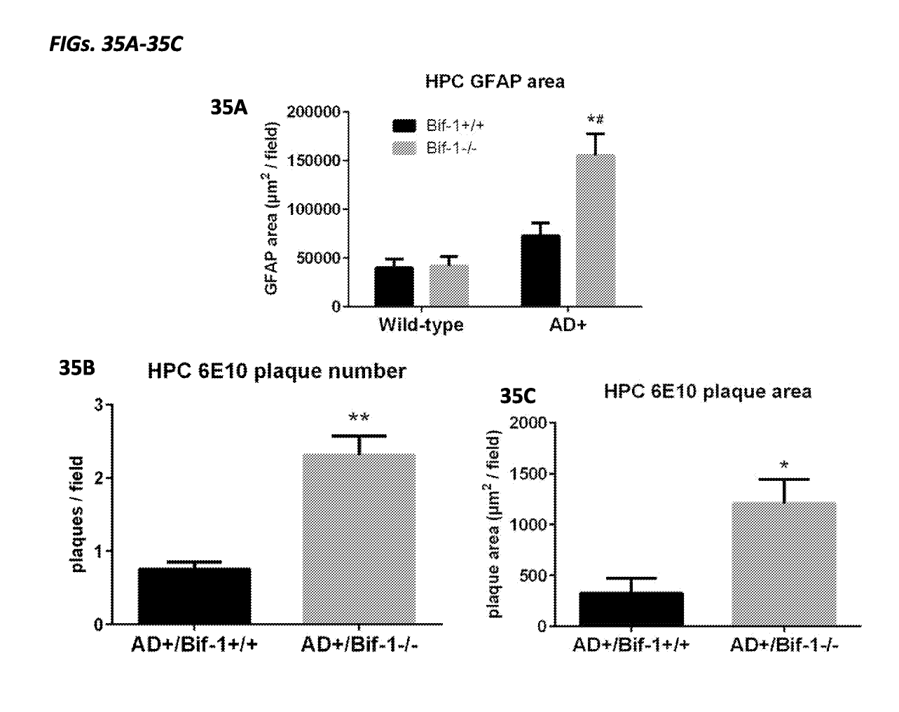

FIGS. 36A-36C are graphs showing that loss of Bif-1 enhances the presence and size of amyloid plaques, a hallmark of Alzheimer's disease pathology.

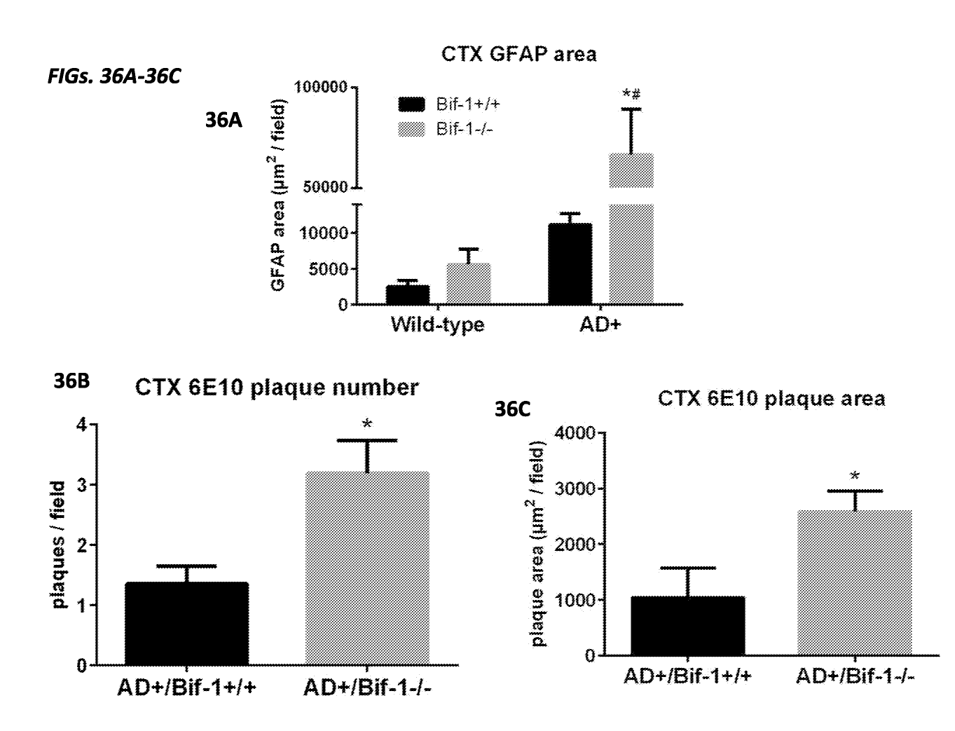

### DETAILED DESCRIPTION

The methods and assays described herein are based, in part, on the discovery that Bax-interacting factor-1 (Bif-1) promotes survival and mitochondrial elongation in neurons and that loss of Bif-1 renders neurons more susceptible to apoptotic stress. Accordingly, provided herein are methods for treating a neurological disease or disorder using compositions comprising Bif-1. Also provided herein are methods and assays relating to predicting sensitivity of a subject to neurological damage. Further, methods are provided herein for reducing amyloid-beta-mediated neurotoxicity in a subject.

Definitions

All scientific and technical terms used in this application have meanings commonly used in the art unless otherwise specified. As used in this application, the following words or phrases have the meanings specified.

As used herein, the term “Bif-1 polypeptide” refers to a polypeptide sequence of Bif-1a, Bif-1b, Bif-1c, Bif-1d, Bif-1e or to a conservative substitution variant or fragment thereof that retains Bif-1 activity as that term is defined herein. By “retaining Bif-1 activity” is meant that a polypeptide retains at least 50% of the Bif-1 activity of the full-length version of the Bif-1 isoform. For example, the Bif-1 fragment retains at least 50% of Bif-1-mediated neuroprotection in an animal model of cerebral infarct (e.g., the middle cerebral artery occlusion model), which is assessed, in part by measuring infarct size. That is, a Bif-1 fragment retains Bif-1 activity if it retains at least 50% of the activity of the full-length Bif-1 as determined by measuring infarct size in Bif-1 fragment treated animals to the infarct size in wildtype, untreated animals or in animals treated with full length Bif-1. Also encompassed by the term “Bif-1 polypeptide” are mammalian homologs of human Bif-1 and conservative substitution variants or fragments thereof that retain Bif-1 activity. In one aspect, such homologs or conservative variants thereof prevent neuronal apoptosis, enhance survival of neurons, or promotes mitochondrial elongation in neurons as measured, for example, as described herein. In one embodiment, a human Bif-1 polypeptide for use with the methods and assays described herein is encoded by the SH3GLB1 gene (Genbank Accession Number NG_{—}030018.1; SEQ ID NO. 6). In another embodiment, a human Bif-1 polypeptide or fragment thereof for use with the methods and assays described herein comprises the sequence of SEQ ID NO.: 1, SEQ ID NO.: 2, SEQ ID NO.:3, SEQ ID NO.: 4,or SEQ ID NO.:5, or a fragment of any of sequences of SEQ ID NO.: 1, SEQ ID NO.: 2, SEQ ID NO.:3, SEQ ID NO.: 4,or SEQ ID NO.:5. The present disclosure is also related to isolated nucleic acids selected from the group consisting of SEQ ID. NOs.: 6-10 or polypeptides/peptides encoded from the isolated nucleic acids selected from the group consisting of SEQ ID. NOs.: 6-10.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that function in a manner similar to a naturally occurring amino acid Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another polypeptide may have one or more mutations relative to the starting polypeptide, e.g., one or more amino acid residues which have been substituted with another amino acid residue or which has one or more amino acid residue insertions or deletions. A polypeptide “derived” from another polypeptide will retain therapeutically or physiologically relevant biological activity of the polypeptide from which it is derived. Relevant activity in this context includes, for example, neuron-specific activity e.g., neuron-specific protection from apoptosis or toxicity induced by stress. By “retain” in such context is meant at least 50% retention, preferably at least 60%, at least 70%, at least 80%, at least 90%, at least 95%, at least 99% or even 100% or greater retention.

A polypeptide can comprise an amino acid sequence which is not naturally occurring. Such variants necessarily have less than 100% sequence identity or similarity with a starting polypeptide molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity. As with polypeptides derived from another, a variant of this kind will retain a therapeutically or physiologically relevant biological activity of the polypeptide from which it is a variant.

In one embodiment, a polypeptide of the invention consists of, consists essentially of, or comprises an amino acid sequence selected from of SEQ ID NO.: 1, SEQ ID NO.: 2, SEQ ID NO.:3, SEQ ID NO.: 4, or SEQ ID NO.:5, and functionally active variants thereof In one embodiment, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in SEQ ID NO.:2. In one embodiment, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence set forth in SEQ ID NO.:2. In one embodiment, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence set forth in of SEQ ID NO.: 1, SEQ ID NO.: 2, SEQ ID NO.:3, SEQ ID NO.: 4,or SEQ ID NO.:5. The Bif-1 polypeptides described herein can comprise conservative amino acid substitutions at one or more amino acid residues, e.g., at essential or non-essential amino acid residues but will retain a therapeutically or physiologically relevant activity of a Bif-1 polypeptide as that term is described herein. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in a conservative substitution variant, a nonessential amino acid residue in a Bif-1 polypeptide is preferably replaced with another amino acid residue from the same side chain family.

The term “variant” as used herein refers to a polypeptide or nucleic acid that is “substantially similar” to a wild-type Bif-1 polypeptide or polynucleic acid. A molecule is said to be “substantially similar” to another molecule if both molecules have substantially similar structures (i.e., they are at least 50% similar in amino acid sequence as determined by BLASTp alignment set at default parameters) and are substantially similar in at least one therapeutically or physiologically relevant function (e.g., effect on neuronal survival, infarct size in a middle cerebral artery occlusion (MCAO) model, or neuronal mitochondrial length). A variant differs from the naturally occurring polypeptide or nucleic acid by one or more amino acid or nucleic acid deletions, additions, substitutions or side-chain modifications, yet retains one or more specific functions or biological activities of the naturally occurring molecule Amino acid substitutions include alterations in which an amino acid is replaced with a different naturally-occurring or a non-conventional amino acid residue. Some substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non-conservative,” in which an amino acid residue which is present in a peptide is substituted with an amino acid having different properties (e.g., substituting a charged or hydrophobic amino acid with an uncharged or hydrophilic amino acid), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). Polynucleotide changes can result in amino acid substitutions, additions, deletions, fusions and truncations in the polypeptide encoded by the reference sequence. Variants can also include insertions, deletions or substitutions of amino acids in the peptide sequence. To be therapeutically useful, such variants will retain a therapeutically or physiologically relevant activity as that term is used herein.

Without wishing to be bound by theory, the inventors discovered, in part, that the inclusion of both exons 6L and exon 7 together are important for the neuroprotective function of a Bif-1 polypeptide. The inventors have generated data showing that expression of Bif-1e (which lacks exon 6 but contains exon 7) does not confer protection in neurons against abeta toxicity while in the same study Bif-1c does. Bif-1d (which contains all of exon 6, thus 6L but lacks exon 7) also lacks neuroprotective activity against Abeta. Therefore, in some embodiments, for the treatment of a neurological disease as described herein, a Bif-1 polypeptide comprising exon 6L and exon 7 together are administered to induce neuroprotection in neurons. Without wishing to be bound by theory, Bif-1a, Bif-1b, and Bif-1c all promote the same degree of cell death when expressed in mouse embryo fibroblasts as described herein in the Examples section. Thus, while exons 6L and 7 are important in neurons, they do not seem to play an important role in non-neuronal cells. Without wishing to be bound by theory, these data indicate that neurons may have unique binding proteins that can distinguish between the different Bif-1 isoforms.

The term “derivative” as used herein refers to peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990). The term “functional” when used in conjunction with “derivative” or “variant” refers to a polypeptide which possesses a therapeutically or physiologically relevant biological activity that is substantially similar to a biological activity of the entity or molecule of which it is a derivative or variant. By “substantially similar” in this context is meant that at least 50% of the relevant or desired biological activity of a corresponding wild-type peptide is retained. In the instance of promoting neuronal survival, for example, an activity retained would be reducing infarct size in an MCAO model; preferably the variant retains at least 60%, at least 70%, at least 80%, at least 90%, at least 95%, at least 100% or even higher (i.e., the variant or derivative has greater activity than the wild-type), e.g., at least 110%, at least 120%, or more activity compared to the activity of wild-typ. Additional activity parameters include, e g., inhibition of neuronal cell death, or promotion of neuronal cell survival relative to the wild-type polypeptide.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

In one embodiment, a polynucleotide of the invention consists of, consists essentially of, or comprises a nucleotide sequence encoding SEQ ID NO.: 1, SEQ ID NO.: 2, SEQ ID NO.:3, SEQ ID NO.: 4,or SEQ ID NO.:5and functionally active variants thereof In an embodiment, a polynucleotide includes a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding SEQ ID NO.: 1, SEQ ID NO.: 2, SEQ ID NO.:3, SEQ ID NO.: 4,or SEQ ID NO.:5. In one embodiment, a polynucleotide includes a nucleotide sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous nucleotides of a nucleotide sequence encoding of SEQ ID NO.: 1, SEQ ID NO.: 2, SEQ ID NO.:3, SEQ ID NO.: 4,or SEQ ID NO.:5.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors, DNA or RNA expression vectors encapsulated in liposomes, and certain eukaryotic cells, such as producer cells.

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue- or cell-specific regulatory sequences). Expression vectors are specifically contemplated for use in expressing Bif-1 polypeptides in methods described herein. Furthermore, for embodiments in which RNA interference is contemplated, RNA interfering agents can be delivered by way of a vector comprising a regulatory sequence to direct synthesis of the siRNAs at specific intervals, or over a specific time period. It will be appreciated by those skilled in the art that the design of an expression vector can depend on such factors as the choice of the target cell, the level of expression desired, and the like.

The expression vectors of the invention can be introduced into target cells to thereby produce Bif-1 polypeptides or siRNA molecules as desired. In one embodiment, a DNA template, e.g., a DNA template encoding the siRNA molecule directed against Bif-1, can be ligated into an expression vector under the control of RNA polymerase III (Pol III), and delivered to a target cell. Pol III directs the synthesis of small, noncoding transcripts which 3′ ends are defined by termination within a stretch of 4-5 thymidines. Accordingly, DNA templates can be used to synthesize, in vivo, both sense and antisense strands of siRNAs which effect RNAi (Sui, et al. (2002) PNAS 99(8):5515).

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

The term “pharmaceutically acceptable” refers to compounds and compositions which can be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990).

As used herein, “a” or “an” means at least one, unless clearly indicated otherwise. As used herein, to “prevent” or “protect against” a condition or disease means to hinder, reduce or delay the onset or progression of the condition or disease.

As used herein, the terms “treat” “treatment” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a neurological disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a neurological disease or disorder, diminishment of extent of the neurological disease or disorder, stabilized (i.e., not worsening) state of the neurological disease or disorder, delay or slowing of progression of the disease, amelioration or palliation of the neurological disease state, and remission (whether partial or total). The term “treatment” of a disease also includes providing at least partial relief from the symptoms or side-effects of the disease (including palliative treatment).

In one embodiment, as used herein, the term “prevention” or “preventing” when used in the context of a subject refers to stopping, hindering, and/or slowing down the development of a neurological disease or disorder.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., a neurological or neurodegenerative disease). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of a therapeutic agent is used, that is, the highest safe dose according to sound medical judgment. It will be understood by those of ordinary skill in the art, however, that a lower dose or tolerable dose that is effective can be administered for medical reasons, psychological reasons or for virtually any other reason.

In one embodiment, a therapeutically effective amount of a pharmaceutical formulation, or a composition described herein for a method of treating a neurological disease or disorder is an amount sufficient to reduce the level of at least one symptom of the neurological disease or disorder (e.g., impaired cognitive function, dementia, neuronal cell death, abnormal mitochondrial characteristics, accumulation of A-beta protein, etc.) as compared to the level in the absence of the compound, the combination of compounds, the pharmaceutical composition/formulation or the composition. In other embodiments, the amount of the composition administered is preferably safe and sufficient to treat, delay the development of a neurological disease or disorder, and/or delay onset of the disease. In some embodiments, the amount can thus cure or result in amelioration of the symptoms of the neurological disease or disorder, slow the course of the disease, slow or inhibit a symptom of the disease, or slow or inhibit the establishment or development of secondary symptoms of the neurological disease or disorder. For example, an effective amount of a composition described herein inhibits further symptoms associated with a neurological disease or disorder, causes a reduction in one or more symptoms associated with a neurological disease or disorder. While effective treatment need not necessarily initiate complete regression of the disease, such effect would be effective treatment. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased” ,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “statistically significant” or “significantly” refers to statistical significance and generally means two standard deviations (2SD) or more above or below normal or a reference. The term refers to statistical evidence that there is a difference. It is defined as the probability of making a decision to reject the null hypothesis when the null hypothesis is actually true. The decision is often made using the p-value.

As used herein, the term “injury or neural injury” is intended to include a damage which directly or indirectly affects the normal functioning of the CNS. For example, the injury can be damage to retinal ganglion cells; a traumatic brain injury; a stroke related injury; a cerebral aneurism related injury; a spinal cord injury, including monoplegia, diplegia, paraplegia, hemiplegia and quadriplegia; a neuroproliferative disorder or neuropathic pain syndrome. Examples of CNS injuries or disease include TBI, stroke, concussion (including post-concussion syndrome), cerebral ischemia, brain injuries secondary to nerve agents, cyanide, toxic concentrations of oxygen, neurotoxicity due to CNS malaria or treatment with anti-malaria agents, trypanosomes, malarial pathogens, and other CNS traumas.

As used herein, the term “stroke” is art recognized and is intended to include sudden diminution or loss of consciousness, sensation, and voluntary motion caused by obstruction (e.g. by a blood clot) of an artery of the brain or loss of blood flow to a region of the brain caused by rupture of a blood vessel in the brain.

As used herein, the term “Traumatic Brain Injury” is art recognized and is intended to include the condition in which, a traumatic blow to the head causes damage to the brain, often without penetrating the skull. Usually, the initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF).

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Neurological Diseases and Disorders

As used herein the term “neurological disease or disorder” refers to any disease or disorder involving the brain, spinal cords, nerves, or neurons in a subject. The term “neurological disease or disorder” encompasses neurodegenerative diseases, CNS disorders, as well as brain injury and trauma. CNS disorders include disorders of the central nervous system as well as disorders of the peripheral nervous system. CNS disorders include, but are not limited to, brain and spinal cord injuries, cerebrovascular ischemia, dementia, traumatic brain injury, stroke, post-stroke, post-traumatic brain injury, small-vessel cerebrovascular disease, and neurological disorders: for example neuropathy, neurotrauma, organophosphate poisoning, depression, schizophrenia, anxiety disorders, epilepsy and bipolar disorder and cognitive-related disorders such as dementia and memory loss.

The term “neurodegenerative disease” as used herein refers to any number of central nervous system disorders characterized by a gradual and progressive loss of neural tissue and/or neural tissue function. Examples of neurodegenerative diseases include, but are not limited to Alzheimer's Disease, Parkinson's Disease, vascular dementia and the like.

Additional neurodegenerative diseases contemplated for treatment using the methods and compositions described herein include, for example, age-related memory impairment, agyrophilic grain dementia, Parkinsonism-dementia complex of Guam, neurological effects of auto-immune conditions (e.g. Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, Dementia Pugilistica, neurofibrillary tangles with calcification, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoffs syndrome, mild cognitive impairment, monomeric amyotrophy, motor neuron diseases, multiple system atrophy, multiple sclerosis and other demyelinating conditions (e.g. leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyngeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

Additional neurodegenerative diseases contemplated for treatment as described herein include other dementias not listed above, such as but without limitation, other mixed dementia, frontotemporal dementia, Pick's Disease, progressive supranuclear palsy (PSP), Parkinson's Disease with associated dementia, corticobasal degeneration, multiple system atrophy, HIV-induced dementia, white matter disease-associated dementias, mild cognitive impairment (MCI).

### Alzheimer's Disease

Provided herein are methods for treating or preventing Alzheimer's disease comprising administering a therapeutically effective amount of a composition comprising a Bif-1 polypeptide or a nucleic acid encoding a Bif-1 polypeptide to a subject in need thereof

Alzheimer's disease (AD) is a progressive disease resulting in senile dementia. See e.g., Selkoe, TINS 16, 403-409 (1993); Hardy et al., WO 92/13069; Selkoe, J. Neuropathol. Exp. Neurol. 53, 438-447 (1994); Duff et al., Nature 373, 476-477 (1995); Games et al., Nature 373, 523 (1995). Alzheimer's disease is often divided into two categories: (i) late onset, which occurs in advanced age (65+ years) and (ii) early onset, which develops well before the senile period, i.e., between 35 and 60 years. In both types of disease, the pathology is the same but the β abnormalities tend to be more severe and widespread in cases beginning at an earlier age. The disease is characterized at the macroscopic level by significant brain shrinkage away from the cranial vault as seen in MRI images as a direct result of neuronal loss and by two types of macroscopic lesions in the brain, senile plaques and neurofibrillary tangles. Senile plaques are areas comprising disorganized neuronal processes up to 150 μm across and extracellular amyloid deposits, which are typically concentrated at the center and visible by microscopic analysis of sections of brain tissue. Neurofibrillary tangles are intracellular deposits of tau protein consisting of two filaments twisted about each other in pairs.

The principal constituent of the plaques is a peptide termed Aβ or β-amyloid peptide. Aβ peptide is an internal fragment of 39-43 amino acids of a precursor protein termed amyloid precursor protein (APP). Aβ is generated by processing of a larger protein APP by two enzymes, termed β and γ secretases (see Hardy, TINS 20, 154 (1997)). Several mutations within the APP protein have been correlated with the presence of Alzheimer's disease. See, e.g., Goate et al., Nature 349, 704) (1991) (valine⁷¹⁷to isoleucine); Chartier Harlan et al. Nature 353, 844 (1991)) (valine⁷¹⁷ to glycine); Murrell et al., Science 254, 97 (1991) (valine⁷¹⁷ to phenylalanine); Mullan et al., glycine); Murrell et al., Science 254, 97 (1991) (valine⁷¹⁷ to phenylalanine); Mullan et al., Nature Genet. 1, 345 (1992) (a double mutation changing lysine⁵⁹⁵-methionine⁵⁹⁶ to asparagine⁵⁹⁵-leucine⁵⁹⁶). Such mutations are thought to cause Alzheimer's disease by increased or altered processing of APP to Aβ, particularly processing of APP to increased amounts of the long form of Aβ (i.e., Aβ 1-42 and Aβ 1-43). Mutations in other genes, such as the presenilin genes, PS1 and PS2, are thought indirectly to affect processing of APP to generate increased amounts of long form Aβ (see Hardy, TINS 20, 154 (1997)). These observations indicate that Aβ, and particularly its long form, is a causative element in Alzheimer's disease.

Aβ has the unusual property that it can fix and activate both classical and alternate complement cascades. In particular, it binds to Clq and ultimately to C3bi. This association facilitates binding to macrophages leading to activation of B cells. In addition, C3bi breaks down further and then binds to CR2 on B cells in a T cell dependent manner leading to a 10,000 increase in activation of these cells. This mechanism causes Aβ to generate an immune response in excess of that of other antigens.

Most therapeutic strategies for Alzheimer's disease are aimed at reducing or eliminating the deposition of Aβ42 in the brain, typically via reduction in the generation of Aβ42 from APP and/or some means of lowering existing Aβ42 levels from sources that directly contribute to the deposition of this peptide in the brain (De Felice and Ferreira, 2002). A partial list of aging-associated causative factors in the development of sporadic Alzheimer's disease includes a shift in the balance between Aβ peptide production and its clearance from neurons that favors intracellular accumulation, increased secretion of Aβ peptides by neurons into the surrounding extracellular space, increased levels of oxidative damage to these cells, and global brain hypoperfusion and the associated compensatory metabolic shifts in affected neurons (Cohen et al., 1988; Higgins et al., 1990; Kalaria, 2000; Nalivaevaa et al., 2004; Teller et al., 1996; Wen et al., 2004).

In some embodiments, agents that activate Bif-1 expression and/or activity as disclosed herein are also useful in the treatment of other neurodegenerative disorders or cognitive impairment disorders in general: for example, dementia, depression, confusion, Creutzfeldt-Jakob or mad cow disease, loss of motor coordination, multiple sclerosis, Parkinson's disease, Pick disease and other brain storage disorders (e.g., amyloidosis, gangliosidosis, lipid storage disorders, mucopolysaccharidosis), syncope, and vascular dementia. It is also contemplated herein that treatment can be directed to a subject who is not affected with symptoms of a neurodegenerative disease, for example, to improve cognitive function. The efficacy of treatment can be determined by methods known to those of skill in the art, for example, by monitoring cerebral blood flow (CBF), monitoring blood-brain barrier (BBB) function, measuring the presence of Tau or Aβ in the CSF.

If so desired, one can determine a baseline value of, for example the level of beta amyloid in the CSF of a subject before administering a dosage of agent, and comparing this with a value for beta amyloid in the CSF after treatment. A decrease, for example a 10% decrease in the level of beta amyloid in the CSF indicates a positive treatment outcome (i.e., that administration of the agent has achieved or augmented a decrease in beta amyloid in the CSF). If the value for level of beta amyloid in the CSF does not change significantly, or increases, a negative treatment outcome is indicated. In general, subjects undergoing an initial course of treatment with an agent are expected to show a decrease in beta amyloid in the CSF with successive dosages of an agent as described herein.

In other methods to determine efficacy of treatment, a control value (i.e., a mean and standard deviation) of beta amyloid is determined for a control population. Typically the individuals in the control population have not received prior treatment and do not suffer from Alzheimer's disease. Measured values of beta amyloid in the CSF in a subject after administering an agent that increases Bif-1 activity and/or expression as disclosed herein are then compared with the control value. A decrease in the beta amyloid in the CSF of the subject relative to the control value (i.e. a decrease of at least 10% of beta amyloid in a subject) signals a positive treatment outcome. A lack of significant decrease signals a negative treatment outcome.

In other methods, a control value of, for example beta amyloid in the CSF is determined from a control population of subjects who have undergone treatment with a therapeutic agent that is effective at reducing beta amyloid in the CSF. Measured values of CSF beta amyloid in the subject are compared with the control value.

Diagnosis of Alzheimer's disease

Subjects amenable to treatment using the methods as disclosed herein include subjects at risk of a neurodegenerative disease, for example Alzheimer's Disease, but not showing symptoms, as well as subjects showing symptoms of a neurodegenerative disease, for example subjects with symptoms of Alzheimer's Disease.

Subjects can be screened for their likelihood of having or developing Alzheimer's Disease based on a number of biochemical and genetic markers. Genetic abnormality in a few families has been traced to chromosome 21 (St. George-Hyslop et al., Science 235:885-890, 1987). For example, mutations in the APP gene, particularly mutations at position 717 and positions 670 and 671 referred to as the Hardy and Swedish mutations respectively (see Hardy, TINS, supra) can be used to assess risk of Alzheimer's disease. Other markers of risk are mutations in the presenilin genes, PS1 and PS2, and ApoE4, family history of Alzheimer's Disease, hypercholesterolemia or atherosclerosis. Subjects with APP, PS1 or PS2 mutations are highly likely to develop Alzheimer's disease. ApoE is a susceptibility gene, and subjects with the e4 isoform of ApoE (ApoE4 isoform) have an increased risk of developing Alzheimer's disease. Test for subjects with ApoE4 isoform are disclosed in U.S. Patent 6,027,896, which is incorporated in its entirety herein by reference.

One can also diagnose a subject with increased risk of developing Alzheimer's disease on the basis of a simple eye test, where the presence of cataracts and/or Abeta in the lens identifies a subject with increased risk of developing Alzheimer's Disease. Methods to detect Alzheimer's disease include using a quasi-elastic light scattering device (Goldstein et al., Lancet. 2003; 12; 361:1258-65) from Neuroptix, using Quasi-Elastic Light Scattering (QLS) and Fluorescent Ligand Scanning (FLS) and a Neuroptix™ QEL scanning device, to enable non-invasive quantitative measurements of amyloid aggregates in the eye, to examine and measure deposits in specific areas of the lens as an early diagnostic for Alzheimer's disease. Methods to diagnose a subject at risk of developing Alzheimer's disease using such a method of non-invasive eye test are disclosed in e.g., U.S. Pat. No. 7,107,092.

Individuals presently suffering from Alzheimer's disease can be recognized from characteristic dementia, as well as the presence of risk factors described above. In addition, a number of diagnostic tests are available for identifying individuals who have AD. These include measurement of CSF tau and Ax3b242 levels. Elevated tau and decreased Ax3b242 levels signify the presence of Alzheimer's Disease.

There are two alternative “criteria” which are utilized to clinically diagnose Alzheimer's Disease: the DSM-IIIR criteria and the NINCDS-ADRDA criteria (which is an acronym for National Institute of Neurological and Communicative Disorders and Stroke (NINCDS) and the Alzheimer's Disease and Related Disorders Association (ADRDA); see McKhann et al., Neurology 34:939-944, 1984). Briefly, the criteria for diagnosis of Alzheimer's Disease under DSM-IIIR include (1) dementia, (2) insidious onset with a generally progressive deteriorating course, and (3) exclusion of all other specific causes of dementia by history, physical examination, and laboratory tests. Within the context of the DSM-IIIR criteria, dementia is understood to involve “a multifaceted loss of intellectual abilities, such as memory, judgment, abstract thought, and other higher cortical functions, and changes in personality and behavior.” (DSM-IIR, 1987).

In contrast, the NINCDS-ADRDA criteria sets forth three categories of Alzheimer's Disease, including “probable,” “possible,” and “definite” Alzheimer's Disease. Clinical diagnosis of “possible” Alzheimer's Disease may be made on the basis of a dementia syndrome, in the absence of other neurologic, psychiatric or systemic disorders sufficient to cause dementia. Criteria for the clinical diagnosis of “probable” Alzheimer's Disease include (a) dementia established by clinical examination and documented by a test such as the Mini-Mental test (Foldstein et al., J. Psych. Res. 12:189-198, 1975); (b) deficits in two or more areas of cognition; (c) progressive worsening of memory and other cognitive functions; (d) no disturbance of consciousness; (e) onset between ages 40 and 90, most often after age 65; and (f) absence of systemic orders or other brain diseases that could account for the dementia. The criteria for definite diagnosis of Alzheimer's Disease include histopathologic evidence obtained from a biopsy, or after autopsy. Since confirmation of definite Alzheimer's Disease requires histological examination from a brain biopsy specimen (which is often difficult to obtain), it is rarely used for early diagnosis of Alzheimer's Disease.

One can also use quantitative electroencephalographic analysis (EEG) to diagnose Alzheimer's Disease. This method employs Fourier analysis of the beta, alpha, theta, and delta bands (Riekkinen et al., “EEG in the Diagnosis of Early Alzheimer's Disease,” Alzheimer's Disease, Current Research in Early Diagnosis, Becker and Giacobini (eds.), pp. 159-167, 1990) for diagnosis of Alzheimer's Disease.

One can also diagnose Alzheimer's Disease by quantifying the degree of neural atrophy, since such atrophy is generally accepted as a consequence of Alzheimer's Disease. Examples of these methods include computed tomographic scanning (CT), and magnetic resonance imaging (MRI) (Leedom and Miller, “CT, MRI, and NMR Spectroscopy in Alzheimer's Disease,” Alzheimer's Disease, Current Research in Early Diagnosis, Becker and Giacobini (eds.), pp. 297-313, 1990).

One can also diagnose Alzheimer's Disease by assessing decreased cerebral blood flow or metabolism in the posterior temporoparietal cerebral cortex by measuring decreased blood flow or metabolism by positron emission tomography (PET) (Parks and Becker, “Positron Emission Tomography and Neuropsychological Studies in Dementia,” Alzheimer's Disease's, Current Research in Early Diagnosis, Becker and Giacobini (eds.), pp. 315-327, 1990), single photon emission computed tomography (SPECT) (Mena et al., “SPECT Studies in Alzheimer's Type Dementia Patients,” Alzheimer's Disease, Current Research in Early Diagnosis, Becker and Giacobini (eds.), pp. 339-355, 1990), and xenon inhalation methods (Jagust et al., Neurology 38:909-912; Prohovnik et al., Neurology 38:931-937; and Waldemar et al., Senile Dementias: II International Symposium, pp. 399407, 1988).

One can also immunologically diagnose Alzheimer's disease (Wolozin, “Immunochemical Approaches to the Diagnosis of Alzheimer's Disease,” Alzheimer's Disease, Current Research in Early Diagnosis, Becker and Giacobini (eds.), pp. 217-235, 1990). Wolozin and coworkers (Wolozin et al., Science 232:648-650, 1986) produced a monoclonal antibody “Alz50,” that reacts with a 68-kDa protein “A68,” which is expressed in the plaques and neuron tangles of patients with Alzheimer's disease. Using the antibody A1z50 and Western blot analysis, A68 was detected in the cerebral spinal fluid (CSF) of some Alzheimer's patients and not in the CSF of normal elderly patients (Wolozin and Davies, Ann. Neurol. 22:521-526, 1987).

It follows that one can determine efficacy of Alzheimer's disease treatment on the basis of improvement in any of the indicators noted above.

Diagnosis of Dementia and/or Memory Impairment

Current standard practice can be used to diagnose various types of dementia and, once diagnosed, to monitor the progression of the disease over an extended period of time. One such method includes at least one of the following; (i) a memory assessment, (ii) an extensive neuropsychological exam, (iii) an examination by a geriatric neurologist and (iv) MRI imaging of the brain. Disease progression is documented by changes in these parameters over time. In some embodiments, changes in the parameters of at least one of these assessments can be used to diagnose dementia or memory impairment and/or assess the efficacy of a composition comprising Bif-1 in the subject over time.

A memory assessment can be used, such as the program of the UMDNJ New Jersey Institute for Successful Aging. Adult patients with complaint of short term memory and/or cognitive decline are seen in the Memory Assessment Program, comprising evaluation by Geriatric Neurology, Neuropsychology and Social services. Patients can be both self-referred or directed from community clinicians and physicians on the suspicion of a possible or probable memory disorder or dementia. In such a memory assessment, at the time of the initial evaluation, all of the evaluations such as (i) memory assessment (ii) an extensive neuropsychological exam, (iii) an examination by a geriatric neurologist and (iv) MRI imaging of the brain are performed the same day. The neuropsychology assessment captures a broad inventory of cognitive function which aids in determining the array and severity of deficits. These include assessments of Judgment, Insight, Behavior, Orientation, Executive Control, General Intellectual Functioning, Visualspatial Function, Memory and New Learning Ability. Depression, if present, is identified. The neurological evaluation captures the history of cognitive alteration as well as the general medical history, and typically a complete neurological exam is performed. The neurological examination can also comprise laboratory studies to exclude reversible causes of dementia including Vitamin B12, Folate, Basic Metabolic Profile, CBC, TSH, ALT, AST, C-reactive protein, serum homocysteine, and RPR. The brain imaging provides a structural brain image, such as brain MRI, although one can use other brain imaging methods known by persons of ordinary skill in the art. The data matrix of history, neuropsychologic tests, neurologic examination, laboratory studies and neuroimaging is used to formulate the diagnosis.

Dementia diagnosis can be based upon the guidelines of the American Academy of Neurology Practice Parameter published in 2001. Diagnosis of vascular dementia can be based on State of California AD Diagnostic and Treatment Centers criteria.

### Brain Injury and Trauma

In one embodiment, the methods and assays described herein are used in the treatment of brain injury or trauma.

Traumatic brain injury (TBI), also known as intracranial injury, occurs when an external force traumatically injures the brain. TBI can be classified based on severity, mechanism (closed or penetrating head injury), or other features (e.g., occurring in a specific location or over a widespread area).

TBI is a major cause of death and disability worldwide, especially in children and young adults. Males sustain traumatic brain injuries more frequently than do females. Causes include falls, vehicle accidents, and violence. Brain trauma can occur as a consequence of a focal impact upon the head, by a sudden acceleration/deceleration within the cranium or by a complex combination of both movement and sudden impact. In addition to the damage caused at the moment of injury, brain trauma causes secondary injury, a variety of events that take place in the minutes and days following the injury. These processes, which include alterations in cerebral blood flow and the pressure within the skull, contribute substantially to the damage from the initial injury. TBI can cause a host of physical, cognitive, social, emotional, and behavioral effects, and outcome can range from complete recovery to permanent disability or death.

### Cerebrovascular Accidents and Stroke

It is contemplated herein that the methods and assays comprising administration of an agent that enhances Bif-1 activity and/or expression can be used in the treatment of, or prevention of neurological damage due to a cerebrovascular accident, or a transient ischemic attack etc.

As used herein the terms “stroke” and “cerebrovascular accident” are used interchangeably herein to refer to a rapid loss of brain function due to disturbance in the blood supply to the brain. This can be due to ischemia (lack of blood flow) caused by blockage (thrombosis, arterial embolism), hypoxia (e.g., due to a lack of sufficient oxygen), or a hemorrhage. As a result, the affected area of the brain cannot function, which might result in an inability to move one or more limbs on one side of the body, inability to understand or formulate speech, or an inability to see one side of the visual field.

A stroke is considered to be a medical emergency and can cause permanent neurological damage and death. Risk factors for stroke include old age, high blood pressure, previous stroke or transient ischemic attack (TIA), diabetes, high cholesterol, tobacco smoking and atrial fibrillation.

In an ischemic stroke, blood supply to part of the brain is decreased, leading to dysfunction of the brain tissue in the region fed by the affected arteries. Ischemia can occur due to (i) thrombosis (obstruction of a blood vessel by a blood clot forming locally), (ii) Embolism (obstruction due to an embolus from elsewhere in the body), (iii) Systemic hypoperfusion (general decrease in blood supply, e.g., in shock), or (iv) venous thrombosis.

The Oxford Community Stroke Project classification (OCSP, also known as the Bamford or Oxford classification) classifies acute ischemic stroke based on the initial symptoms and the extent of the symptoms. A stroke episode can be classified as total anterior circulation infarct (TACI), partial anterior circulation infarct (PACI), lacunar infarct (LACI) or posterior circulation infarct (POCI). These four entities predict the extent of the stroke, the area of the brain affected, the underlying cause, and the prognosis. The TOAST (Trial of Org 10172 in Acute Stroke Treatment) classification is based on clinical symptoms as well as results of further investigations; on this basis, a stroke is classified as being due to (1) thrombosis or embolism due to atherosclerosis of a large artery, (2) embolism of cardiac origin, (3) occlusion of a small blood vessel, (4) other determined cause, (5) undetermined cause (two possible causes, no cause identified, or incomplete investigation).

A transient ischemic attack (TIA) is a transient episode of neurologic dysfunction caused by ischemia (loss of blood flow) that occurs without acute infarction (tissue death).

TIAs have the same underlying cause as strokes: a disruption of cerebral blood flow (CBF), and are frequently referred to as “mini-strokes.” TIAs and strokes cause the same symptoms, such as contralateral paralysis (opposite side of body from affected brain hemisphere) or sudden weakness or numbness. A TIA may cause sudden dimming or loss of vision (amaurosis fugax), aphasia, slurred speech (dysarthria) and mental confusion. But unlike a stroke, the symptoms of a TIA can resolve within a few minutes or 24 hours. Brain injury may still occur in a TIA lasting only a few minutes.

Improving Cognition, Learning and/or Memory

The methods and assays described herein are also contemplated for use in improving cognition, enhancing learning, and/or improving memory. Such changes in overall neurologic health can improve the quality of life in e.g., elderly patients, children and adults with learning disabilities, patients having a neurodegenerative disease or a brain trauma, and in those with mild to extreme dementia. “Cognition” is used to refer to a group of mental processes that includes attention, memory, producing and understanding language, learning, reasoning, problem solving, and decision making.

### Animal Models of Neurodegenerative Disease(s)

Animal models of vascular dementia include, for example occlusion of carotid arteries in rats. See, e.g., Sarti et al., Behavioral Brain Research 136: 13-20 (2002). Thus, cerebrovascular white matter lesions can be experimentally induced in the rat brain as a result of chronic cerebral hypoperfusion. This model is created by permanent occlusion of both common carotid arteries. This model produces physiological changes as well as learning and memory problems. For example, the gait performance of rats with occluded arteries declines over time in comparison with baseline, for example, at and 90 days, rats with bilateral common carotid artery occlusion have decreased performances on object recognition and Y maze spontaneous alternation test in comparison with sham-operated rats. Thus, this rat model of experimental chronic cerebral hypoperfusion by permanent occlusion of the bilateral common carotid arteries is useful as a model for significant learning impairments along with rarefaction of the white matter. This model is a useful tool to assess the effectiveness of agents that activate Bif-1 expression and/or activity on the pathophysiology of chronic cerebral hypoperfusion, and to provide data for determining optimal dosages and dosage regimens for preventing the cognitive impairment and white matter lesions in patients.

The effectiveness of agents that increase Bif-1 activity and/or expression for treating or preventing dementia can therefore be determined by observing the gait performance, memory, learning abilities and the incidence and severity of white matter lesions in rats with carotid artery occlusions. Similarly, the dosage and administration schedule of compositions that enhance Bif-1 expression and/or activity can be adjusted pursuant to the memory and learning abilities of human patients being treated for vascular dementia.

The suitability of an agent comprising Bif-1 as described herein for the treatment of a neurodegenerative disease can be assessed in any of a number of animal models for neurodegenerative disease. For example, mice transgenic for an expanded polyglutamine repeat mutant of ataxin-1 develop ataxia typical of spinocerebellar ataxia type 1 (SCA-1) are known (Burright et al., 1995, Cell 82: 937-948; Lorenzetti et al., 2000, Hum. Mol. Genet. 9: 779-785; Watase, 2002, Neuron 34: 905-919). Additional animal models, for example, for Alzheimer's disease (Hsiao, 1998, Exp. Gerontol, 33: 883-889; Hsiao et al., 1996, Science 274: 99-102), Parkinson's disease (Kim et al., 2002, Nature 418: 50-56), amyotrophic lateral sclerosis (Zhu et al., 2002, Nature 417: 74-78), Pick's disease (Lee & Trojanowski, 2001, Neurology 56 (Suppl. 4): S26-S30, and spongiform encephalopathies (He et al., 2003, Science 299: 710-712) can be used to evaluate the efficacy of the agents comprising Bif-1.

Animal models are not limited to mammalian models. For example, Drosophila strains provide accepted models for a number of neurodegenerative disorders (reviewed in Fortini & IBonini, 2000, Trends Genet. 16: 161-167; Zoghbi & Botas, 2002, Trends Genet. 18: 463-471). These models include not only flies bearing mutated fly genes, but also flies bearing human transgenes, optionally with targeted mutations. Among the Drosophila models available are, for example, spinocerebellar ataxias (e.g., SCA-1 (see, e.g., WO 02/058626), SCA-3 (Warrick et al., 1998, Cell 93: 939-949)), Parkinson's disease (Feany et al, 2000, Nature 404: 394-398; Auluck et al. , 2002, Science 295: 809-810), age-dependent neurodegeneration (Genetics, 2002,161:4208), Alzheimer's disease (Selkoe et al., 1998, Trends Cell Biol. 8: 447-453; Ye et al., 1999, J. Cell Biol. 146: 1351-1364), amyotrophic lateral sclerosis (Parkes et al., 1998, Nature Genet. 19: 171-174), and adrenoleukodystrophy.

Animals administered the compositions described herein are evaluated for symptoms relative to animals not administered such compositions. A measurable change in the severity of a symptom (i.e., a decrease in at least one symptom, i.e. 10% or greater decrease), or a delay in the onset of a symptom, in animals treated with a composition comprising Bif-1 versus untreated animals is indicative of therapeutic efficacy.

One can assess the animals for memory and learning, for instance by performing behavioral testing. One can use any behavioral test for memory and learning commonly known by person of ordinary skill in the art, for but not limited to the Morris water maze test for rodent animal models. A measurable increase in the ability to perform the Morris water maze test in animals administered a Bif-1 agent versus untreated animals is indicative of therapeutic efficacy.

### Bax Interacting Factor-1 (Bif-1)

Bax-interacting factor-1 (Bif-1), also known as endophilin B1 or SH3GLB1, was originally identified as a proapoptotic protein that binds to and activates Bax in response to apoptotic stress. In non-neuronal cells overexpression of Bif-1 promotes apoptosis, while knockdown of Bif-1 suppressed cytochrome c release and apoptosis. Consistent with the ability of endophilins to induce membrane curvature, Bif-1 has also been implicated in the regulation of mitochondrial morphology, as knockdown or expression of a dominant negative form of Bif-1 resulted in elongated mitochondria in HeLa cells.

The endophilin B1 gene gives rise to at least three splice variants, endophilin B1a, which shows a widespread tissue distribution, and endophilins B1b and B1c, which are brain-specific. Modregger et al. (Journal of Biological Chemistry 278:4160-4167 (2003)) describes a “brain specific form” of Bif-1 and provides a schematic that maps out the location of the major exons involved in splicing which includes exons 6S, 6L and 7. The paper notes that exon 6S is a part of exon 6L and is 16 amino acids shorter than exon 6L. Modregger et al. does not describe the novel form of Bif-1 that the inventors discovered, which includes exon 7 and lacks exon 6.

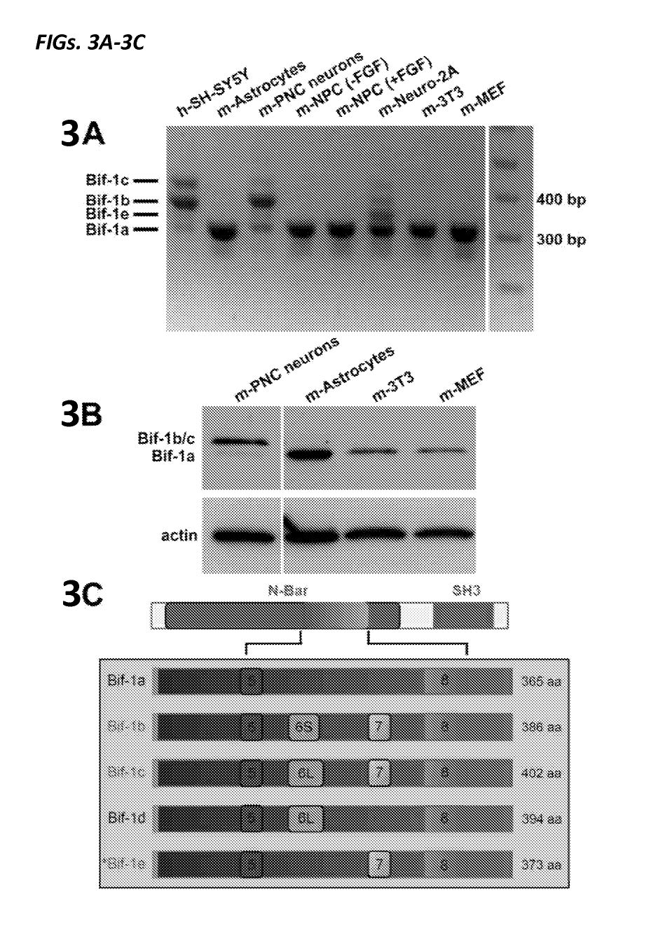

As described herein in the Examples section, the inventors demonstrated that only neurons and neuroblastoma cells expressed longer isoforms of Bif-1 message and protein (e.g., Bif-1b, Bif-1c). In contrast, all other cell types tested, including fibroblasts, astrocytes, and neuronal progenitor cells expressed only the shortest isoform, Bif-1a. The inventors also note that Bif-1b is preferentially expressed over the other neuron-specific forms. A diagram depicting the different Bif-1 isoforms is shown in FIG. 3C. The ubiquitously expressed Bif-1a is the shortest, lacking exons 6 and 7, while Bif-1c is the longest and contains both exons 6 and 7. Bif-1b differs from Bif-1c in that it contains a short (39 bp) form of exon 6 (6S) rather than the long (89 bp) form (6L). The inventors have also identified an additional isoform, termed Bif-1d, which contains full length exon 6 but lacks exon 7. Neurons and neuroblastoma cells expressed an additional isoform that contains exon 7 but lacks exon 6, which is referred to herein as Bif-1e.

Bif-1 does not have significant homology to other Bcl-2 family members, but rather contains an N-terminal Bin-Amphiphysin-Rvs (BAR) domain, typically involved in membrane dynamics, and a C-terminal SH3 domain. It is contemplated herein that a peptide corresponding to the BAR domain or SH3 domain or any other functional domain of Bif-1 can be used with the methods and assays as described herein.

It is contemplated herein that any one of the Bif-1 isoforms, or a combination thereof, can be used with the methods and assays described herein. That is, any Bif-1 polypeptide, or a combination of Bif-1 polypeptides can be administered to a subject to treat a neurological disease or disorder, or to prevent neurological damage in a subject determined to be at risk for neurological damage. In some embodiments, a neuron-specific Bif-1 isoform is employed as described herein (e.g., Bif-1b, Bif-1c, Bif-1d, or Bif-1e). In one embodiment, the Bif-1 isoform used with the methods and assays described herein is Bif-1b and/or Bif-1c—these isoforms are expected to be most beneficial for the treatment of neurological conditions. Also contemplated for use with the methods and assays described herein are isolated nucleic acid sequences selected from the group consisting of SEQ ID. NOs.: 6-10 or polypeptides/peptides encoded from the isolated nucleic acid sequences selected from the group consisting of SEQ ID. NOs.: 6-10.

SEQ ID NO.: 1
endophilin B1 [Homo sapiens]. Isoform A, 365 aa
mnimdfnvkk laadagtfls ravqfteekl gqaektelda
hlenllskae ctkiwtekim kqtevllqpn pnarieefvy
ekldrkapsr innpellgqy midagtefgp gtaygnalik
cgetqkrigt adreliqtsa lnfltplrnf iegdyktiak
erkllqnkrl dldaaktrlk kakaaetrns seqelritqs
efdrqaeitr lllegissth ahhlrclndf veaqmtyyaq
cyqymldlqk qlgsfpsnyl snnnqtsvtp vpsvlpnaig
ssamastsgl vitspsnlsd lkecsgsrka rvlydydaan
stelsllade vitvfsvvgm dsdwlmgerg nqkgkvpity
lelln
SEQ ID NO.: 2
endophilin B1 [Homo sapiens]. Isoform B, 386 aa
mnimdfnvkk laadagtfls ravqfteekl gqaektelda
hlenllskae ctkiwtekim kqtevllqpn pnarieefvy
ekldrkapsr innpellgqy midagtefgp gtaygnalik
cgetqkrigt adreliqtsa lnfltplrnf iegdyktiak
erkllqnkrl dldaaktrlk kakaaetrns qlnsarlegd
nimiwaeevt kseqelritg sefdrqaeit rlllegisst
hahhlrclnd fveaqmtyya qcyqymldlq kqlgsfpsny
lsnnnqtsvt pvpsvlpnai gssamastsg lvitspsnls
dlkecsgsrk arvlydydaa nstelsllad evitvfsvvg
mdsdwlmger gnqkgkvpit ylelln
SEQ ID NO.: 3
endophilin B1 [Homo sapiens]. Isoform C, 402 aa
mnimdfnvkk laadagtfls ravqfteekl gqaektelda
hlenllskae ctkiwtekim kqtevllqpn pnarieefvy
ekldrkapsr innpellgqy midagtefgp gtaygnalik
cgetqkrigt adreliqtsa lnfltplrnf iegdyktiak
erkllqnkrl dldaaktrlk kakaaetrns qlnsarlegd
nimvnfsyml nflhvkwlki waeevtkseq elritqsefd
rcqeitrlll egissthahh lrclndfvea qmtyyaqcyq
ymldlqkqlg sfpsnylsnn nqtsvtpvps vlpnaigssa
mastsglvit spsnlsdlke csgsrkarvl ydydaanste
lslladevit vfsvvgmdsd wlmgergnqk gkvpitylelln
SEQ ID NO.: 4
endophilin B1 [Homo sapiens]. Isoform D, 394 aa
mnimdfnvkk laadagtfls ravqfteekl gqaektelda
hlenllskae ctkiwtekim kqtevllqpn pnarieefvy
ekldrkapsr innpellgqy midagtefgp gtaygnalik
cgetqkrigt adreliqtsa lnfltplrnf iegdyktiak
erkllqnkrl dldaaktrlk kakaaetrns qlnsarlegd
nimvnfsyml nflhvkwlks eqelritqse fdrqaeitrl
llegisstha hhlrclndfv eaqmtyyaqc yqymldlqkq
lgsfpsnyls nnnqtsvtpv psvlpnaigs samastsglv
itspsnlsdl kecsgsrkar vlydydaans telslladev
itvfsvvgmd sdwlmgergn qkgkvpityl elln
SEQ ID NO.: 5
endophilin B1 [Homo sapiens]. Isoform e, 373 aa
mnimdfnvkk laadagtfls ravqfteekl gqaektelda
hlenllskae ctkiwtekim kqtevllqpn pnarieefvy
ekldrkapsr innpellgqy midagtefgp gtaygnalik
cgetqkrigt adreliqtsa lnfltplrnf iegdyktiak
erkllqnkrl dldaaktrlk kakaaetrns iwaeevtkse
qelritqsef drqaeitrll legissthah hlrclndfve
aqmtyyaqcy qymldlqkql gsfpsnylsn nnqtsvtpvp
svlpnaigss amastsglvi tspsnlsdlk ecsgsrkary
lydydaanst elslladevi tvfsvvgmds dwlmgergnq
kgkvpityle lln

### Biological Samples

A biological sample can be obtained from any organ or tissue in the individual to be tested, provided that the biological sample comprises Bif-1.

In some embodiments, a biological sample is treated to remove cells or other biological particulates. Methods for removing cells from a blood or other biological sample are well known in the art and can include e.g., centrifugation, ultrafiltration, immune selection, or sedimentation etc. Proteins and nucleic acids can be detected from a biological sample or a sample that has been treated as described above or as known to those of skill in the art.

Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a cell lysate sample, a crude cell lysate sample, a production sample, a drug preparation sample, a biological molecule production sample, a protein preparation sample, a lipid preparation sample, a carbohydrate preparation sample, or a combination of such samples. For the methods described herein, it is preferred that a biological sample is from whole blood, plasma, cerebral spinal fluid, serum, and/or urine. In one embodiment, the biological sample is cerebrospinal fluid.

### In Vitro Detection of Bif-1

Provided herein are a variety of assay formats that can be used to determine the concentration or level of Bif-1 in a biological sample. Examples of assay formats include known techniques such as Western blot analysis, radioimmunoassay (hereinafter referred to as “RIA”), immunoradiometric assay (IRMA), chemiluminescent immunoassays, such as enzyme-linked immunosorbent assay (hereinafter referred to as “ELISA”), multiplex bead assays, a fluorescence antibody method, passive haemagglutination, mass spectrometry (such as MALDI/TOF (time-of-flight), SELDI/TOF), liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, and tandem mass spectrometry HPLC. Some of the immunoassays can be easily automated by the use of appropriate instruments such as the IM x™ (Abbott, Irving, Tex.) for a fluorescent immunoassay and Ciba Corning ACS 180™ (Ciba Corning, Medfield, Mass.) for a chemiluminescent immunoassay.

RIA and ELISA provide the benefit of detection sensitivity, rapidity, accuracy, possible automation of procedures, and the like, for the determination of the concentration or level of a Bif-1 polypeptide or a fragment thereof. Radioimmunoassay (Kashyap, M. L. et al., J. Clin. Invest., 60:171-180 (1977)) is a technique in which a detection antibody can be used after labeling with a radioactive isotope such as ¹²⁵I. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

The most common enzyme immunoassay is the “Enzyme-Linked Immunosorbent Assay (ELISA). There are different forms of ELISA which are well known to those skilled in the art, e.g. standard ELISA, competitive ELISA, and sandwich ELISA. The standard techniques for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904. ELISA is a technique for detecting and measuring the concentration of an antigen, such as a Bif-1 polypeptide or fragment thereof, using a labeled (e.g. enzyme linked) form of the antibody. In a “sandwich ELISA”, an antibody is linked to a solid phase (i.e. a microtiter plate) and exposed to a biological sample containing antigen (e.g. a Bif-1 polypeptide or fragment thereof). The solid phase is then washed to remove unbound antigen. A labeled antibody (e.g. enzyme linked) is then bound to the plate bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured. In a “competitive ELISA”, a specific concentration of an antibody specific for a Bif-1 polypeptide or fragment thereof is incubated with a biological sample. The Bif-1-antibody mixture is then contacted with a solid phase (e.g. a microtiter plate) that is coated with a Bif-1 polypeptide. The Bif-1 polypeptide present in the sample, the less free antibody that will be available to bind to the solid phase. A labeled (e.g., enzyme linked) secondary antibody is then added to the solid phase to determine the amount of primary antibody bound to the solid phase.

Detection of Bif-1 Using Mass Spectrometry

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces; ” U.S. Pat. No. 6,107,623, entitled “Methods and Apparatus for Tandem Mass Spectrometry; ” U.S. Pat. No. 6,268,144, entitled “DNA Diagnostics Based On Mass Spectrometry; ” U.S. Pat. No. 6,124,137, entitled “Surface-Enhanced Photolabile Attachment And Release For Desorption And Detection Of Analytes; ” Wright et al., “Proteinchip surface enhanced laser desorption/ionization (SELDI) mass spectrometry: a novel protein biochip technology for detection of prostate cancer biomarkers in complex protein mixtures,” Prostate Cancer and Prostatic Diseases 2: 264-76 (1999); and Merchant and Weinberger, “Recent advancements in surface-enhanced laser desorption/ionization-time of flight-mass spectrometry,” Electrophoresis 21: 1164-67 (2000), each of which is hereby incorporated by reference in its entirety, including all tables, figures, and claims. Mass spectrometry methods are well known in the art and have been used to quantify and/or identify biomolecules, such as proteins and hormones (see, e.g., Li et al., (2000), Tibtech. 18:151-160; Starcevic et. al., (2003), J. Chromatography B, 792: 197-204; Kushnir M M et. al. (2006), Clin. Chem. 52:120-128; Rowley et al. (2000), Methods 20: 383-397; and Kuster and Mann (1998), Curr. Opin. Structural Biol. 8: 393-400). Further, mass spectrometric techniques have been developed that permit at least partial de novo sequencing of isolated proteins. Chait et al., (1993), Science, 262:89-92; Keough et al., (1999), Proc. Natl. Acad. Sci. USA. 96:7131-6; reviewed in Bergman (2000), EXS 88:133-44. Various methods of ionization are known in the art. For examples, Atmospheric Pressure Chemical Ionisation (APCI) Chemical Ionisation (CI) Electron Impact (EI) Electrospray Ionisation (ESI) Fast Atom Bombardment (FAB) Field Desorption/Field Ionisation (FD/FI) Matrix Assisted Laser Desorption Ionisation (MALDI) and Thermospray Ionisation (TSP) In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait). In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material. For additional information regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094. Detection and quantification of the Bif-1 polypeptide or fragment thereof will typically depend on the detection of signal intensity. For example, in certain embodiments, the signal strength of peak values from spectra of a first sample and a second sample can be compared (e.g., visually, by computer analysis etc.), to determine the relative amounts of Bif-1. Software programs such as the Biomarker Wizard program (Ciphergen Biosystems, Inc., Fremont, Calif.) can be used to aid in analyzing mass spectra. The mass spectrometers and their techniques are well known to those of skill in the art. The various assays are described herein in terms of the detection of Bif-1 in e.g., cerebrospinal fluid. However, it should be understood that the assays can be readily adapted to detect other analytes as needed for various other embodiments and in various other sample types, such as blood, plasma, or urine.

Provided herein are prognostic methods useful for determining a proper course of treatment for a patient having, or at risk of having, a neurological disease or disorder or trauma. A course of treatment refers to the therapeutic measures taken for a patient after diagnosis or after treatment for injury.

Also provided herein are commercial kits for the detection and prognostic evaluation of Bif-1. The kit can be in any configuration well known to those skilled in the art and is useful for performing one or more of the methods described herein for the detection of a Bif-1 polypeptide or fragment thereof. The kits are convenient in that they supply many, if not all, of the essential reagents for conducting an assay for the detection of Bif-1 in a biological sample, such as described herein. In addition, the assay can be performed simultaneously with a standard or multiple standards included in the kit, such as a predetermined amount of a recombinant Bif-1 polypeptide so that the results of the test can be quantified or validated.

In one embodiment, the kit comprises a means for detecting levels of a Bif-1 polypeptide or fragment thereof in a biological sample. The kit may comprise a “dipstick” with a Bif-1 binding agent immobilized thereon, which specifically binds a Bif-1 polypeptide or fragment thereof. Specifically bound Bif-1 can then be detected using, for example, a second antibody that is detectably labeled with a calorimetric agent or radioisotope.

In other embodiments, the assay kits may contain components for competitive and non-competitive assays, radioimmunoassay (RIA), multiplex bead assays, bioluminescence and chemiluminescence assays, fluorometric assays, sandwich assays, immunoradiometric assays, dot blots, enzyme linked assays including ELISA, microtiter plates, or immunocytochemistry. For each kit the range, sensitivity, precision, reliability, specificity, and reproducibility of the assay are established by means well known to those skilled in the art.

### In Vivo Detection of Bif-1 in the Brain

It is also contemplated herein that Bif-1 expression and/or activity in the brain is assessed using in vivo imaging techniques. Thus, in some embodiments, an agent that binds a Bif-1 polypeptide or fragment thereof (e.g., a peptide or antibody) is coupled or conjugated to one or more imaging moieties. As utilized herein, “imaging moiety” (I) means a moiety which can be utilized to increase contrast between a region of the brain (e.g., a neuron) and the intracellular expression of Bif-1 by e.g., radiography, positron-emission tomography, magnetic resonance imaging, direct or indirect visual inspection. Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.).

In general, imaging agents can be conjugated to the anti-Bif-1 binding agent by any suitable technique, with appropriate consideration of the need for pharmokinetic stability and reduced overall toxicity to the patient. An imaging agent can be coupled to a suitable antibody moiety either directly or indirectly (e.g. via a linker group). A direct reaction between an imaging agent and an antibody is possible when each possesses a functional group capable of reacting with the other. For example, a nucleophilic group, such as an amino or sulfhydryl group, is capable of reacting with a carbonyl-containing group, such as an anhydride or an acid halide, or with an alkyl group containing a good leaving group (e.g., a halide). Alternatively, a suitable chemical linker group can be used. A linker group can function as a spacer to distance an antibody from an imaging agent in order to avoid interference with binding capabilities. A linker group can also serve to increase the chemical reactivity of a substituent on a moiety or an antibody, and thus increase the coupling efficiency. An increase in chemical reactivity can also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups can be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues. As an alternative coupling method, imaging moieties can be coupled to the anti-Bif-1 antibody moiety through a an oxidized carbohydrate group at a glycosylation site, as described in U.S. Pat. Nos. 5,057,313 and 5,156,840. Yet another alternative method of coupling the binding agent moiety to the imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the imaging moiety.

It may be desirable to couple more than one imaging moiety to an antibody. By poly-derivatizing the anti-Bif-1 antibody, a Bif-1 binding agent can be made useful as a contrasting agent for several visualization techniques, or a therapeutic antibody may be labeled for tracking by a visualization technique. In one embodiment, multiple molecules of an imaging moiety are coupled to one Bif-1 binding molecule. In another embodiment, more than one type of moiety can be coupled to one binding agent. Regardless of the particular embodiment, immunoconjugates with more than one moiety may be prepared in a variety of ways. For example, more than one moiety may be coupled directly to an antibody molecule, or linkers which provide multiple sites for attachment (e.g., dendrimers) can be used. Alternatively, a carrier with the capacity to hold more than one imaging moiety can be used.

A carrier may bear the agents in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier may also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful for imaging moiety conjugation to anti-Bif-1 antibody moieties for use with the methods and assays described herein, as a sufficient amount of the imaging moiety (dye, magnetic resonance contrast reagent, etc.) for detection is more easily associated with the antibody moiety.

Carriers and linkers specific for radionuclide agents (for use as positron-emission imaging moieties) include radiohalogenated small molecules and chelating compounds. For example, U.S. Pat. No. 4,735,792 discloses representative radiohalogenated small molecules and their synthesis. A radionuclide chelate can be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis. Such chelation carriers are also useful for magnetic spin contrast ions for use in magnetic resonance imaging tumor visualization methods, and for the chelation of heavy metal ions for use in radiographic visualization methods.

Preferred radiographic moieties for use as imaging moieties with the methods and assays described herein include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods as described herein. Examples of such compositions which may be utilized for x-ray radiography are described in U.S. Pat. No. 5,709,846, incorporated fully herein by reference. Such moieties may be conjugated to the anti-Bif-1 antibody moiety through an acceptable chemical linker or chelation carrier. In addition, radionuclides which emit radiation capable of penetrating the skull can be useful for scintillation imaging techniques. Suitable radionuclides for conjugation include 99Tc, 111In, and 67Ga. Positron emitting moieties for use in the present invention include 18F, which can be easily conjugated by a fluorination reaction with the anti-Bif-1 binding moiety according to the method described in U.S. Pat. No. 6,187,284.

Preferred magnetic resonance contrast moieties include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates can be conjugated to a Bif-1 binding agent through a suitable chemical linker.

Optically visible moieties for use as imaging moieties include fluorescent dyes, or visible-spectrum dyes, visible particles, and other visible labeling moieties. Fluorescent dyes such as fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the anti-Bif-1 antibody. Alternatively, visible particles, such as colloidal gold particles or latex particles, can be coupled to the anti-Bif-1 antibody moiety via a suitable chemical linker.

### Reference Values

The terms “reference value,” “reference level,” “reference sample,” and “reference” are used interchangeably herein and refer to the level of Bif-1 expression in a known sample against which another sample (i.e., one obtained from a subject lacking detectable neurological disease) is compared. A reference value is useful for determining the amount of Bif-1 expression or the relative increase/decrease of such expressional levels/ratios in a biological sample. A reference value serves as a reference level for comparison, such that samples can be normalized to an appropriate standard in order to infer the presence, absence or extent of neurological disease or sensitivity to neurological damage in a subject.

In one embodiment, a biological standard is obtained at an earlier time point (e.g., prior to the onset of a neurological disease) from the same individual that is to be tested or treated as described herein. Alternatively, a standard can be from the same individual having been taken at a time after the onset or diagnosis of a neurological disease or disorder. In such instances, the reference value can provide a measure of the efficacy of treatment. It can be useful to use as a reference for a given patient a level or ratio from a sample taken after diagnosis of a neurological disease but before the administration of any therapy to that patient.

Alternatively, a reference value can be obtained, for example, from a known biological sample from a different individual (e.g., not the individual being tested) that is e.g., substantially free of neurological disease. A known sample can also be obtained by pooling samples from a plurality of individuals to produce a reference value or range of values over an averaged population, wherein a reference value represents an average level of Bif-1 expression and/or activity among a population of individuals (e.g., a population of individuals lacking neurological disease). Thus, the level Bif-1 in a reference value obtained in this manner is representative of an average level of this marker in a general population of individuals lacking neurological disease. An individual sample is compared to this population reference value by comparing expression of Bif-1 from a sample relative to the population reference value. Generally, a decrease in the amount of Bif-1 (e.g., a reference obtained from subjects lacking neurological disease) indicates or predicts an increased sensitivity to neurological damage caused by e.g., stress, while an increase in the amount of Bif-1 indicates or predicts that the subject is more resistant to neurological damage. The converse is contemplated in cases where a reference value is obtained from a population of subjects having a neurological disease or disorder. It should be noted that there is often variability among individuals in a population, such that some individuals will have higher levels of Bif-1 expression, while other individuals have lower levels of expression. However, one skilled in the art can make logical inferences on an individual basis regarding the detection and treatment of neurological disease as described herein.

In one embodiment, a range of values for Bif-1 in e.g., cerebrospinal fluid can be defined for a plurality of individuals with or without detectable neurological disease. Provided that the number of individuals in each group is sufficient, one can define a range of Bif-1 values for each population. These values can be used to define cut-off points for selecting a therapy or for monitoring progression of disease. Thus, one of skill in the art can determine the level of Bif-1 and compare the value to the ranges in each particular sub-population to aid in determining the status of disease and the recommended course of treatment. Such value ranges are analogous to e.g., HDL and LDL cholesterol levels detected clinically. For example, LDL levels below 100 mg/dL are considered optimal and do not require therapeutic intervention, while LDL levels above 190 mg/dL are considered ‘very high’ and will likely require some intervention. One of skill in the art can readily define similar parameters for Bif-1 expression in a variety of neurological statuses. These value ranges can be provided to clinicians, for example, on a chart, programmed into a PDA etc.

A standard comprising a reference value or range of values can also be synthesized. A known amount of Bif-1 (or a series of known amounts) can be prepared within the typical expression range for Bif-1 that is observed in a general population. In one embodiment, a recombinant Bif-1, such as Bif-1b or Bif-1c is used as a standard for generating a reference value or set of values. This method has an advantage of being able to compare the extent of disease in one or more individuals in a mixed population. This method can also be useful for subjects who lack a prior sample to act as a reference value or for routine follow-up post-diagnosis. This type of method can also allow standardized tests to be performed among several clinics, institutions, or countries etc.

### Systems for Determining Bif-1 Polypeptide Concentration

Other aspects described herein also provide for systems (and computer readable media for causing computer systems) to perform a method for determining the expression value of a Bif-1 polypeptide or fragment thereof (e.g., Bif-1b, Bif-1c).

In some aspects, embodiments as disclosed herein can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including any suitable combination of the foregoing. In one embodiment, the computer readable storage media is not a carrier signal or other such transient computer readable media.

Computer-readable data embodied on one or more computer-readable media can define instructions, for example, as part of one or more programs, that, as a result of being executed by a computer, instruct the computer to perform one or more of the functions described herein, and/or various embodiments, variations and combinations thereof Such instructions can be written in any of a plurality of programming languages, for example, Java, J#, Visual Basic, C, C#, C++, Fortran, Pascal, Eiffel, Basic, COBOL assembly language, and the like, or any of a variety of combinations thereof The computer-readable media on which such instructions are embodied can reside on one or more of the components of either of a system, or a computer readable storage medium described herein, can be distributed across one or more of such components.

The computer-readable media can be transportable such that the instructions stored thereon can be loaded onto any computer resource to implement the embodiments or aspects described herein. In addition, it should be appreciated that the instructions stored on the computer-readable medium, described above, are not limited to instructions embodied as part of an application program running on a host computer. Rather, the instructions can be embodied as any type of computer code (e.g., software or microcode) that can be employed to program a computer to implement aspects of methods and assays described herein. The computer executable instructions can be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination system #40, a storage device #30, a comparison module #80, and a display module #110. The functional modules can be executed on one, or multiple, computers, or by using one, or multiple, computer networks. The determination system has computer executable instructions to provide e.g., fluorescence information in computer readable form.

The determination system #40, can comprise any system for detecting a signal from one or more protein binding agents, e.g., a fluorescently labeled antibody that binds a Bif-1 polypeptide or fragment thereof Such systems can include flow cytometry systems, fluorescence assisted cell sorting systems, fluorescence microscopy systems (e.g., fluorescence microscopy, confocal microscopy), any ELISA detection system and/or any Western blotting detection system.

The information determined in the determination system can be read by the storage device #30. As used herein the “storage device” is intended to include any suitable computing or processing apparatus or other device configured or adapted for storing data or information. Examples of electronic apparatus suitable for use with the methods and assays described herein include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon expression level, mass spectrum data, or protein level information. Such information can be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising expression level information.

In one embodiment, the reference data stored in the storage device to be read by the comparison module is mass spectrum data or fluorescence emission data obtained from an ELISA determination system #40.

The “comparison module” #80 can use a variety of available software programs and formats for the comparison operative to compare fluorescence data determined in the determination system to reference samples and/or stored reference data. In one embodiment, the comparison module is configured to use pattern recognition techniques to compare information from one or more entries to one or more reference data patterns. The comparison module can be configured using existing commercially-available or freely-available software for comparing patterns, and can be optimized for particular data comparisons that are conducted. The comparison module provides computer readable information related to normalized expression level of a Bif-1 polypeptide or fragment thereof, the sensitivity of an individual to neurological damage, efficacy of treatment in an individual, and/or method for treating an individual.

The comparison module, or any other module of the systems described herein, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application can include a configuration file which contains pointers and addresses to the various software entities that comprise the server as well as the various external and internal databases which must be accessed to service user requests. The Configuration file also directs requests for server resources to the appropriate hardware--as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the systems described herein, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that can be stored and output as requested by a user using a display module #110. The content based on the comparison result, can be a expression value or mass spectrum data compared to a reference that shows whether an individual has an increased risk of neurological damage in response to stress or disease.

In one embodiment of the systems described herein, the content based on the comparison result is displayed on a computer monitor #120. In one embodiment of the systems described herein, the content based on the comparison result is displayed through printable media #130, #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

Also provided herein are systems (and computer readable media for causing computer systems) to perform methods for assessing whether an individual has, or is at risk of having, an increased sensitivity to neurological damage.

Systems and computer readable media described herein are merely illustrative embodiments for performing methods of assessing whether an individual has an increased risk of neurological damage in response to stress, and are not intended to limit the scope of the invention. Variations of the systems and computer readable media described herein are possible and are intended to fall within the scope of the invention.

The modules of the machine, or those used in the computer readable medium, can assume numerous configurations. For example, a function can be provided on a single machine or distributed over multiple machines.

### Expression Vectors

A nucleic acid encoding a Bif-1 polypeptide can be expressed in a cell (e.g., a neuron) using an expression vector. The term “vector” refers to a carrier DNA molecule into which a nucleic acid sequence can be inserted for introduction into a host cell. An “expression vector” is a specialized vector that contains the necessary regulatory regions needed for expression of a gene of interest in a host cell. In some embodiments the gene of interest is operably linked to another sequence in the vector. In some embodiments, it is preferred that the viral vectors are replication defective, which can be achieved for example by removing all viral nucleic acids that encode for replication. A replication defective viral vector will still retain its infective properties and enters the cells in a similar manner as a replicating vector, however once admitted to the cell a replication defective viral vector does not reproduce or multiply.

Many viral vectors or virus-associated vectors are known in the art. Such vectors can be used as carriers of a nucleic acid construct into the cell. Constructs can be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector can be incorporated into the cell's genome. The constructs can include viral sequences for transfection, if desired. Alternatively, the construct can be incorporated into vectors capable of episomal replication, e.g. EPV and EBV vectors. The inserted material of the vectors described herein can be operatively linked to an expression control sequence when the expression control sequence controls and regulates the transcription and translation of that polynucleotide sequence. The term “operatively linked” can include having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of an inserted material is under the control of a promoter sequence (or other transcriptional regulatory sequence) which controls the expression of the recombinant gene in a cell-type in which expression is intended. It will also be understood that the inserted material can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

An “inducible promoter” is a promoter that is capable of directly or indirectly activating transcription of one or more DNA sequences or genes in response to a “regulatory agent” (e.g., doxycycline), or a “stimulus” (e.g., heat). In the absence of a “regulatory agent” or “stimulus”, the DNA sequences or genes will not be substantially transcribed. The term “not substantially transcribed” or “not substantially expressed” means that the level of transcription is at least 100-fold lower than the level of transcription observed in the presence of an appropriate stimulus or regulatory agent; preferably at least 200-fold, 300-fold, 400-fold, 500-fold or more. As used herein, the terms “stimulus” and/or “regulatory agent” refers to a chemical agent, such as a metabolite, a small molecule, or a physiological stress directly imposed upon the organism such as cold, heat, toxins, or through the action of a pathogen or disease agent. A recombinant cell containing an inducible promoter can be exposed to a regulatory agent or stimulus by externally applying the agent or stimulus to the cell or organism by exposure to the appropriate environmental condition or the operative pathogen. Inducible promoters initiate transcription only in the presence of a regulatory agent or stimulus. Examples of inducible promoters include the tetracycline response element and promoters derived from the β-interferon gene, heat shock gene, metallothionein gene or any obtainable from steroid hormone-responsive genes. Inducible promoters which can be used in performing the methods as described herein include those regulated by hormones and hormone analogs such as progesterone, ecdysone and glucocorticoids as well as promoters which are regulated by tetracycline, heat shock, heavy metal ions, interferon, and lactose operon activating compounds. For review of these systems see e.g., Gingrich and Roder, 1998, Annu Rev Neurosci 21, 377-405. Tissue specific expression has been well characterized in the field of gene expression and tissue specific and inducible promoters are well known in the art. These promoters are used to regulate the expression of the foreign gene after it has been introduced into the target cell.

The promoter sequence can be a “tissue-specific promoter,” which means a nucleic acid sequence that serves as a promoter, i.e., regulates expression of a selected nucleic acid sequence operably linked to the promoter, and which affects expression of the selected nucleic acid sequence in specific cells, preferably in neurons. The term also covers so-called “leaky” promoters, which regulate expression of a selected nucleic acid primarily in one tissue, but cause expression in other tissues as well. For expression of an exogenous gene specifically in neuronal cells, a neuron-specific enolase promoter can be used (see Forss-Petter et al., 1990, Neuron 5: 187-197). For expression of an exogenous gene in dopaminergic neurons, a tyrosine hydroxylase promoter can be used. For expression in pituitary cells, a pituitary-specific promoter such as POMC may be useful (Hammer et al., 1990, Mol. Endocrinol. 4:1689-97). Other cell specific promoters active in mammalian cells are also contemplated herein. Such promoters provide a convenient means for controlling expression of the exogenous gene in a cell of a cell culture or within a mammal.

In some embodiments, the expression vector is a lentiviral vector. Lentiviral vectors useful for the methods and compositions described herein can comprise a eukaryotic promoter. The promoter can be any inducible promoter, including synthetic promoters that can function as a promoter in a eukaryotic cell. For example, the eukaryotic promoter can be, but is not limited to, ecdysone inducible promoters, E1a inducible promoters, tetracycline inducible promoters etc., as are well known in the art. In addition, the lentiviral vectors used herein can further comprise a selectable marker, which can comprise a promoter and a coding sequence for a selectable trait. Nucleotide sequences encoding selectable markers are well known in the art, and include those that encode gene products conferring resistance to antibiotics or anti-metabolites, or that supply an auxotrophic requirement. Examples of such sequences include, but are not limited to, those that encode thymidine kinase activity, or resistance to methotrexate, ampicillin, kanamycin, chloramphenicol, or zeocin, among many others.

### Delivery of Nucleic Acids Encoding Bif-1

The principles of gene delivery are disclosed by Oldham, R. K. (In: Principles of Biotherapy, Raven Press, N.Y., 1987), and similar texts. Disclosures of the methods and uses for gene therapy are provided by Boggs, S. S. (Int. J. Cell Clon. 8:80-96 (1990)); Karson, E. M. (Biol. Reprod. 42:39-49 (1990)); Ledley, F. D., In: Biotechnology, A Comprehensive Treatise, volume 7B, Gene Technology, VCH Publishers, Inc. NY, pp 399-458 (1989)), all of which references are incorporated herein by reference.

In one embodiment, the nucleic acid encoding Bif-1 can be administered to a patient by any one of several gene therapy techniques known to those of skill in the art. In general, gene therapy can be accomplished by either direct transformation of target cells within the mammalian subject (in vivo gene therapy) or transformation of cells in vitro and subsequent implantation of the transformed cells into the mammalian subject (ex vivo gene therapy).

In one embodiment of the methods described herein, DNA encoding a Bif-1 polypeptide can be introduced into the somatic cells of an animal (particularly mammals including humans) in order to provide a treatment of a disease or condition that responds to the composition. Most preferably, viral or retroviral vectors are employed for this purpose.

Retroviral vectors are a common mode of delivery and in this context are often retroviruses from which viral genes have been removed or altered so that viral replication does not occur in cells infected with the vector. Viral replication functions are provided by the use of retrovirus “packaging” cells that produce the viral proteins required for nucleic acid packaging but that do not produce infectious virus.

Introduction of the retroviral vector DNA into packaging cells results in production of virions that carry vector RNA and can infect target cells, but such that no further virus spread occurs after infection. To distinguish this process from a natural virus infection where the virus continues to replicate and spread, the term transduction rather than infection is often used.

In one embodiment, the methods use a recombinant lentivirus for the delivery and expression of a Bif-1 polypeptide or peptide in either dividing or non-dividing mammalian cells. The HIV-1 based lentivirus can effectively transduce a broader host range than the Moloney Leukemia Virus (MoMLV)-based retroviral systems. Preparation of the recombinant lentivirus can be achieved using the pLenti4/V5-DEST™, pLenti6/V5-DEST™ or pLenti vectors together with ViraPower™ Lentiviral Expression systems from INVITROGEN.

Examples of use of lentiviral vectors for gene therapy for e.g., neurological disorders, are described in the following references and are hereby incorporated by reference in their entirety (Klein, C. and Baum, C. (2004). Hematol. J., 5, 103-111; Zufferey, Ret. al. (1997). Nat. Biotechnol., 15, 871-875; Morizono, K. et. al. (2005). Nat. Med., 11, 346-352; Di Domenico, C. et. al. (2005), Hum. Gene Ther., 16, 81-90; Kim, E. Y., Hong, Y. B., Lai, Z., Kim, H. J., Cho, Y.-H., Brady, R. O. and Jung, S.-C. (2004). Biochem. Biophys. Res. Comm, 318, 381-390).

Non-retroviral vectors also have been used in genetic therapy. One such alternative is the adenovirus (Rosenfeld, M. A., et al., Cell 68:143155 (1992); Jaffe, H. A. et al., Nature Genetics 1:372-378 (1992); Lemarchand, P. et al., Proc. Natl. Acad. Sci. USA 89:6482-6486 (1992)). Major advantages of adenovirus vectors are their potential to carry large segments of DNA (36 Kb genome), a very high titer (10¹¹ particles/ml), ability to infect non-replicating cells, and suitability for infecting tissues in situ. Similarly, herpes viruses may also prove valuable for human gene therapy (Wolfe, J. H. et al., Nature Genetics 1:379-384 (1992)). Of course, any other suitable viral vector can be used for the genetic therapy for the delivery of a nucleic acid encoding Bif-1 as described herein.

The viron used for gene therapy can be any viron known in the art including but not limited to those derived from adenovirus, adeno-associated virus (AAV), retrovirus, and lentivirus. Recombinant viruses provide a versatile system for gene expression studies and therapeutic applications.

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, the methods described herein use a recombinant adeno-associated virus (rAAV) vector for the expression of a Bif-1 polypeptide or peptide, or e.g., a fusion protein including a peptide as described herein. Using rAAV vectors, genes can be delivered into a wide range of host cells including many different human and non-human cell lines or tissues. Because AAV is non-pathogenic and does not elicit an immune response, a multitude of pre-clinical studies have reported excellent safety profiles. rAAVs are capable of transducing a broad range of cell types and transduction is not dependent on active host cell division. High titers, >108 viral particle/ml, are easily obtained in the supernatant and 1011-1012 viral particle/ml can be obtained with further concentration. The transgene is integrated into the host genome, so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, for example, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying a DNA coding sequence for a peptide, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

Although local administration will most likely be preferred, a nucleic acid encoding Bif-1 used in the methods described herein can be delivered systemically via in vivo gene therapy. Systemic treatment involves transfecting target cells with the DNA of interest, i.e. DNA encoding a Bif-1 polypeptide or peptide, expressing the coded peptide/protein in that cell, and the capability of the transformed cell to subsequently secrete the manufactured peptide/protein into the blood.

A variety of methods have been developed to accomplish in vivo transformation including mechanical means (e.g., direct injection of nucleic acid into target cells or particle bombardment), recombinant viruses, liposomes, and receptor-mediated endocytosis (RME) (for reviews, see Chang et al. 1994 Gastroenterol. 106:1076-84; Morsy et al. 1993 JAMA 270:2338-45; and Ledley 1992 J. Pediatr. Gastroenterol. Nutr. 14:328-37).

Another gene transfer method for use in humans is the transfer of plasmid DNA in liposomes directly to human cells in situ (Nabel, E. G., et al., Science 249:1285-1288 (1990)). Plasmid DNA may be easy to certify for use in human gene therapy because, unlike retroviral vectors, it can be purified to homogeneity. In addition to liposome-mediated DNA transfer, several other physical DNA transfer methods, such as those targeting the DNA to receptors on cells by conjugating the plasmid DNA to proteins, have shown promise in human gene therapy (Wu, G. Y., et al., J. Biol. Chem. 266:14338-14342 (1991); Curiel, D. T., et al., Proc. Natl. Acad. Sci. USA, 88:8850-8854 (1991)).

### Bif-1 Binding Agents and Antibodies

In one embodiment, a binding agent (e.g., a peptide) or antibody that binds to e.g., Bif-1 (e.g., Bif-1a, Bif-1b, Bif-1c, Bif-1d, Bif-1e, neuron-specific Bif-1, pan-Bif-1) is used herein in methods and assays for predicting sensitivity of a subject to neurological damage or for treating a neurological disease or disorder.

An “antibody” that can be used according to the methods described herein includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies. Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen.

Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., 1988. While both polyclonal and monoclonal antibodies can be used in the methods described herein, it is preferred that a monoclonal antibody is used where conditions require increased specificity for a particular protein.

Useful monoclonal antibodies and fragments can be derived from any species (including humans) or can be formed as chimeric proteins which employ sequences from more than one species. Human monoclonal antibodies or “humanized” murine antibodies are also used in accordance with the methods and assays described herein. For example, a murine monoclonal antibody can be “humanized” by genetically recombining the nucleotide sequence encoding the murine Fv region (i.e., containing the antigen binding sites) or the complementarity determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction of the possibly of adverse immune reactions. The murine monoclonal antibodies should preferably be employed in humanized form. Antigen binding activity is determined by the sequences and conformation of the amino acids of the six complementarity determining regions (CDRs) that are located (three each) on the light and heavy chains of the variable portion (Fv) of the antibody. The 25-kDa single-chain Fv (scFv) molecule is composed of a variable region (VL) of the light chain and a variable region (VH) of the heavy chain joined via a short peptide spacer sequence. Techniques have been developed to display scFv molecules on the surface of filamentous phage that contain the gene for the scFv. scFv molecules with a broad range of antigenic-specificities can be present in a single large pool of scFv-phage library.

Chimeric antibodies are immunoglobin molecules characterized by two or more segments or portions derived from different animal species. Generally, the variable region of the chimeric antibody is derived from a non-human mammalian antibody, such as a murine monoclonal antibody, and the immunoglobulin constant region is derived from a human immunoglobulin molecule. In some embodiments, both regions and the combination have low immunogenicity as routinely determined

### Small Molecule Modulation of Bif-1 Activity or Expression

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Essentially any small molecule modulator of Bif-1 expression and/or activity (e.g., an activator or inhibitor) can be used in the treatment of certain neurological diseases using the methods described herein. Screening assays are provided herein for identifying candidate small molecule agents that modulate Bif-1 expression and/or activity.

### Nucleic Acid Inhibitors of Bif-1

A powerful approach for inhibiting the expression of selected target polypeptides is through the use of RNA interference agents. RNA interference (RNAi) uses small interfering RNA (siRNA) duplexes that target the messenger RNA encoding the target polypeptide for selective degradation. siRNA-dependent post-transcriptional silencing of gene expression involves cleaving the target messenger RNA molecule at a site guided by the siRNA. “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, 20%, 30%, 40%, 50%, 60%, 70%, 80%, 90%, 95% or 99% or more as compared to the expression of a target gene or the activity or level of the protein encoded by a target gene which has not been targeted by an RNA interfering agent.

The terms “RNA interference agent” and “RNA interference” as they are used herein are intended to encompass those forms of gene silencing mediated by double-stranded RNA, regardless of whether the RNA interfering agent comprises an siRNA, miRNA, shRNA or other double-stranded RNA molecule. “Short interfering RNA” (siRNA), also referred to herein as “small interfering RNA” is defined as an RNA agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent can be a cellular gene or genomic sequence, e.g. the Bif-1 sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target. The siRNA preferably targets only one sequence. Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one can also screen the potential target sequences for similar sequences in the sequence databases to identify potential sequences which can have off-target effects. For example, according to Jackson et al. (Id.), 15, or perhaps as few as 11 contiguous nucleotides, of sequence identity are sufficient to direct silencing of non-targeted transcripts. Therefore, one can initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target human GGT mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the mRNA. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated. The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

In a preferred embodiment, the RNA interference agent is delivered or administered in a pharmaceutically acceptable carrier. Additional carrier agents, such as liposomes, can be added to the pharmaceutically acceptable carrier. In another embodiment, the RNA interference agent is delivered by a vector encoding small hairpin RNA (shRNA) in a pharmaceutically acceptable carrier to the cells in an organ of an individual. The shRNA is converted by the cells after transcription into siRNA capable of targeting, for example, Bif-1.

In one embodiment, the vector is a regulatable vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In one embodiment, the RNA interference agents used in the methods described herein are taken up actively by cells in vivo following intravenous injection, e.g., hydrodynamic injection, without the use of a vector, illustrating efficient in vivo delivery of the RNA interfering agents. One method to deliver the siRNAs is by topical administration in an appropriate pharmaceutically acceptable carrier. Other strategies for delivery of the RNA interference agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs, using a basic peptide by conjugating or mixing the RNA interfering agent with a basic peptide, e.g., a fragment of a TAT peptide, mixing with cationic lipids or formulating into particles. The RNA interference agents, e.g., the siRNAs targeting Bif-1 mRNA, can be delivered singly, or in combination with other RNA interference agents, e.g., siRNAs, such as, for example siRNAs directed to other cellular genes. siRNAs can also be administered in combination with other pharmaceutical agents which are used to treat or prevent diseases or disorders comprising a neurological disease or disorder. Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structure. Within cells, Dicer processes the short hairpin RNA (shRNA) into effective siRNA. The targeted region of the siRNA molecule can be selected from a given target gene sequence, e.g., a Bif-1 coding sequence, beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a siRNA molecule involves identifying the 23 nucleotide sequence motif AA(N19)TT (SEQ. ID. NO. 21) (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al., (2001) supra and Elbashir et al., 2001 supra). Analysis of sequence databases, including but not limited to the NCBI, BLAST, Derwent and GenSeq as well as commercially available oligosynthesis companies such as Oligoengine®, can also be used to select siRNA sequences against EST libraries to ensure that only one gene is targeted.

siRNA sequences to target Bif-1 can also be obtained commercially from e.g., INVITROGEN™, THERMO SCIENTIFIC™, ORIGENE™, among others.

### Delivery of RNA Interfering Agents

Methods of delivering RNA interference agents, e.g., an siRNA, or vectors containing an RNA interference agent, to the target cells, e.g., neurons, or other desired target cells, for uptake include injection of a composition containing the RNA interference agent, e.g., an siRNA, or directly contacting the cell, e.g., a neuron, with a composition comprising an RNA interference agent, e.g., an siRNA. For example, the RNA interference agent can be delivered directly to the brain (e.g., intracranial injection), the spinal cord, or a peripheral nerve ending. In another embodiment, RNA interference agent, e.g., an siRNA can be injected directly into any blood vessel, such as vein, artery, venule or arteriole, via, e.g., hydrodynamic injection or catheterization. Administration can be by a single injection or by two or more injections. The RNA interference agent is delivered in a pharmaceutically acceptable carrier. One or more RNA interference agents can be used simultaneously. In one embodiment, a single siRNA that targets human Bif-1 is used. In another embodiment, one or more siRNAs that target human Bif-1 or neuron-specific Bif-1 is used. In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents, e.g., the siRNAs or shRNAs, can be introduced along with components that perform one or more of the following activities: enhance uptake of the RNA interfering agents, e.g., siRNA, by the cell, e.g., lymphocytes or other cells, inhibit annealing of single strands, stabilize single strands, or otherwise facilitate delivery to the target cell and increase inhibition of the target gene, e.g., Bif-1 or neuron-specific Bif-1. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

### Dosage and Administration

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

In one aspect, the methods described herein provide a method for treating a neurological disease or disorder (e.g., Alzheimer's disease, Parkinson's disease, vascular dementia, among others) in a subject. In one embodiment, the subject can be a mammal. In another embodiment, the mammal can be a human, although the approach is effective with respect to all mammals. The method comprises administering to the subject an effective amount of a pharmaceutical composition comprising a Bif-1 polypeptide or a nucleic acid encoding a Bif-1 polypeptide, in a pharmaceutically acceptable carrier. In some embodiments, the method comprises administering to the subject an effective amount of a pharmaceutical composition comprising an inhibitor of Bif-1, for example, a binding protein, such as an antibody or a peptide. In other embodiments, the inhibitor of Bif-1 comprises a small molecule or an RNA interference molecule (e.g., siRNA, shRNA etc.).

The dosage range for the agent depends upon the potency, and includes amounts large enough to produce the desired effect, e.g., neuroprotection. The dosage should not be so large as to cause unacceptable adverse side effects. Generally, the dosage will vary with the type of inhibitor (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges from 0.001mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 mg/kg body weight to 30 m/kg body weight. Alternatively, the dose range will be titrated to maintain serum levels between 5 μg/mL and 30 m/mL.

Administration of the doses recited above can be repeated for a limited period of time. In some embodiments, the doses are given once a day, or multiple times a day, for example but not limited to three times a day. In another embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in at least one symptom of a neurological disease or disorder (see “Efficacy Measurement” below). Such effective amounts can be gauged in clinical trials as well as animal studies for a given agent.

Agents useful in the methods and compositions described herein can be administered directly to the brain (e.g., intracerebral implantation, intracerebroventricular (ICV) infusion, and convection enhanced diffusion (CED)), into the spinal cord, or to peripheral nerve endings. It is also contemplated herein that the agents can also be delivered intravenously (by bolus or continuous infusion), orally, by inhalation, intranasally, intraperitoneally, intramuscularly, subcutaneously, intracavity, and can be delivered by peristaltic means, if desired, or by other means known by those skilled in the art. In one embodiment it is preferred that the agents for the methods described herein are administered directly to a neuron (e.g., during surgery or by direct injection). The agent can be administered systemically, if so desired.

The composition(s) described herein comprising an agent that enhances Bif-1 activity and/or expression in neurons, can be administered into the epidural space of the spinal cord (e.g., similar to epidural anesthesia), or directly into the cerebrospinal fluid. One of skill in the art will appreciate that the dose of the agent administered into the epidural space will need to be higher than when administered directly to the cerebrospinal fluid (CSF). Delivery of agents to the CSF or the epidural space is well within the abilities of one of ordinary skill in the art.

In another embodiment, the compositions as contemplated herein are administered such that the agents come into contact with a subject's nervous system. In one embodiment, the active agents are administered by introduction into the cerebrospinal fluid of the subject. In certain aspects, the peptide composition is introduced into a cerebral ventricle, the lumbar area, or the cistema magna. In another aspect, the composition is introduced locally, such as into the site of nerve or cord injury, into a site of pain or neural degeneration, or intraocularly to contact neuroretinal cells. In one embodiment, the composition described herein is administered to the subject in the period from the time of, for example, an injury to the CNS up to about 100 hours after the injury has occurred, for example within 24, 12, or 6 hours from the time of injury.

In another embodiment of the invention, the composition is administered into a subject intrathecally. As used herein, the term “intrathecal administration” is intended to include delivering a polypeptide or peptide composition directly into the cerebrospinal fluid of a subject, by techniques including lateral cerebroventricular injection through a burrhole or cistemal or lumbar puncture or the like (described in Lazorthes et al., 1991, and Ommaya A. K., 1984, the contents of which are incorporated herein by reference). The term “lumbar region” is intended to include the area between the third and fourth lumbar (lower back) vertebrae. The term “cistema magna” is intended to include the area where the skull ends and the spinal cord begins at the back of the head. The term “cerebral ventricle” is intended to include the cavities in the brain that are continuous with the central canal of the spinal cord. Administration of an active compound to any of the above mentioned sites can be achieved by direct injection of the active compound formulation or by the use of infusion pumps. Implantable or external pumps and catheter may be used.

An additional means of administration to intracranial tissue involves application of compositions of the invention to the olfactory epithelium, with subsequent transmission to the olfactory bulb and transport to more proximal portions of the brain. Such administration can be by nebulized or aerosolized preparations. In a further embodiment, ophthalmic compositions are used to prevent or reduce damage to retinal and optic nerve head tissues, as well as to enhance functional recovery after damage to ocular tissues. Ophthalmic conditions that may be treated include, but are not limited to, retinopathies (including diabetic retinopathy and retrolental fibroplasia), macular degeneration, ocular ischemia, and glaucoma. Other conditions to be treated with the methods described herein include damage associated with injuries to ophthalmic tissues, such as ischemia reperfusion injuries, photochemical injuries, and injuries associated with ocular surgery, particularly injuries to the retina or optic nerve head by exposure to light or surgical instruments. The ophthalmic compositions can also be used as an adjunct to ophthalmic surgery, such as by vitreal or subconjunctival injection following ophthalmic surgery. The peptide compositions can be used for acute treatment of temporary conditions, or can be administered chronically, especially in the case of degenerative disease. The ophthalmic peptide compositions can also be used prophylactically, especially prior to ocular surgery or noninvasive ophthalmic procedures or other types of surgery.

In one embodiment, the active compound is administered to a subject for an extended period of time. Sustained contact with the peptide composition can be achieved by, for example, repeated administration of the peptide composition over a period of time, such as one week, several weeks, one month or longer. More preferably, the pharmaceutically acceptable formulation used to administer the active compound provides sustained delivery, such as “slow release” of the active compound to a subject. For example, the formulation can deliver the agent or composition for at least one, two, three, or four weeks after the pharmaceutically acceptable formulation is administered to the subject. Preferably, a subject to be treated in accordance with the methods described herein is treated with the active composition for at least 30 days (either by repeated administration or by use of a sustained delivery system, or both).

As used herein, the term “sustained delivery” is intended to include continual delivery of the composition in vivo over a period of time following administration, preferably at least several days, a week, several weeks, one month or longer. Sustained delivery of the active compound can be demonstrated by, for example, the continued therapeutic effect of the composition over time (such as sustained delivery of the agents can be demonstrated by continued improvement in cognition by a subject).

Preferred approaches for sustained delivery include use of a polymeric capsule, a minipump to deliver the formulation, a biodegradable implant, or implanted transgenic autologous cells (as described in U.S. Pat. No. 6,214,622). Implantable infusion pump systems (such as Infusaid; see such as Zierski, J. et al., 1988; Kanoff, R. B., 1994) and osmotic pumps (sold by Alza Corporation) are available in the art. Another mode of administration is via an implantable, externally programmable infusion pump. Suitable infusion pump systems and reservoir systems are also described in U.S. Pat. No. 5,368,562 by Blomquist and U.S. Pat. No. 4,731,058 by Doan, developed by Pharmacia Deltec Inc.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., lesion). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

Delivery Across the Blood Brain Barrier

The blood brain barrier (BBB) is a system-wide membrane barrier that separates the circulating blood from the brain extracellular fluid, and can prevent the uptake of certain circulating drugs, protein therapeutics, RNAi drugs, and gene medicines in the brain. The blood brain barrier comprises endothelial tight junctions surrounding the capillaries that do not exist in the rest of the circulatory system. Endothelial cells of the blood brain barrier restrict the diffusion of microscopic objects (e.g., bacteria) and large or hydrophilic molecules into the cerebrospinal fluid (CSF), while allowing the diffusion of small hydrophobic molecules (O₂, CO₂, hormones). Cells of the barrier actively transport metabolic products such as glucose across the barrier with specific proteins.

Drugs or genes can be delivered to the human brain for the treatment of neurological disease either (a) by injecting the drug or gene directly into the brain, thus bypassing the BBB, or (b) by injecting the drug or gene into the bloodstream so that the drug or gene enters the brain via the transvascular route across the BBB.

Other methods for delivery of agents through the blood brain barrier include, but are not limited to, disruption of the blood brain barrier by osmotic means, biochemical disruption using vasoactive substances such as bradykinin, or by localized exposure to high-intensity focused ultrasound (HIFU). Other methods used to pass compositions through the BBB can employ endogenous transport systems, including carrier-mediated transporters such as glucose and amino acid carriers, receptor-mediated transcytosis for insulin or transferrin, liposomes, nanotechnology, use of peptides, and the blocking of active efflux transporters such as p-glycoprotein. Mannitol can be used in bypassing the BBB.

Methods for delivery of a Bif-1 polypeptide, or a nucleic acid encoding a Bif-1 polypeptide behind the BBB include intracerebral implantation (such as with needles) and convection-enhanced distribution. The Bif-1 polypeptide or nucleic acid encoding the Bif-1 polypeptide can also be delivered directly to the brain (by e.g., intracranial injection), the spinal cord, or a peripheral nerve ending. In another embodiment, the Bif-1 polypeptide, or nucleic acid encoding a Bif-1 polypeptide are injected directly into any blood vessel, such as a vein, artery, venule or arteriole, via e.g., hydrodynamic injection or catheterization.

### Pharmaceutical Compositions

Provided herein are compositions that are useful for treating and preventing neurological cell damage and/or neuronal cell death. In one embodiment, the composition is a pharmaceutical composition. The composition can comprise a therapeutically or prophylactically effective amount of a Bif-1 polypeptide, polynucleotide, or a recombinant virus expressing Bif-1.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The present invention includes, but is not limited to, therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

A pharmaceutical composition can contain DNA encoding one or more of the Bif-1 polypeptides, such that the polypeptide is generated in situ. As noted above, the DNA may be present within any of a variety of delivery systems known to those of ordinary skill in the art, including nucleic acid expression systems, bacterial and viral expression systems. Numerous gene delivery techniques are well known in the art, such as those described by Rolland, 1998, Crit. Rev. Therap. Drug Carrier Systems 15:143-198, and references cited therein. Appropriate nucleic acid expression systems contain the necessary DNA sequences for expression in the patient (such as a suitable promoter and terminating signal. In a preferred embodiment, the DNA can be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., 1989, Proc. Natl. Acad. Sci. USA 86:317-321; Flexner et al., 1989, Ann. My Acad. Sci. 569:86-103; Flexner et al., 1990, Vaccine 8:17-21; U.S. Pat. Nos.4,603,112, 4,769,330, and 5,017,487; WO 89/01973; U.S. Pat. No. 4,777,127; GB 2,200,651; EP 0,345,242; WO 91102805; Berkner, 1988, Biotechniques 6:616-627; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art. The DNA can also be “naked,” as described, for example, in Ulmer et al., 1993, Science 259:1745-1749 and reviewed by Cohen, 1993, Science 259:1691-1692. The uptake of naked DNA can be increased by coating the DNA onto biodegradable beads, which are efficiently transported into the cells.

While any suitable carrier known to those of ordinary skill in the art can be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) can also be employed as carriers for the pharmaceutical compositions of this invention. Suitable biodegradable microspheres are disclosed, for example, in U.S. Pat. Nos. 4,897,268 and 5,075,109. Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions as described herein can be formulated as a lyophilizate. Compounds can also be encapsulated within liposomes using well known technology. The compositions described herein can be administered as part of a sustained release formulation (i.e., a formulation such as a capsule or sponge that effects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide dispersed in a carrier matrix and/or contained within a reservoir surrounded by a rate controlling membrane. Carriers for use within such formulations are biocompatible, and can also be biodegradable; preferably the formulation provides a relatively constant level of active component release. The amount of active compound contained within a sustained release formulation depends upon the site of implantation, the rate and expected duration of release and the nature of the condition to be treated or prevented.

### Efficacy Measurement

The efficacy of a given treatment for a neurological disease or disorder (e.g., Alzheimer's disease, Parkinson's disease, dementia, among others) can be determined by the skilled clinician. However, a treatment is considered “effective treatment,” as the term is used herein, if any one or all of the signs or symptoms of the neurological disease is/are altered in a beneficial manner (e.g., improved cognitive function and/or memory, reduced amyloid beta protein accumulation, reduced neuronal cell death, improved neuronal cell function etc.), other clinically accepted symptoms or markers of disease are improved, or even ameliorated, e.g., by at least 10% following treatment with an agent comprising a Bif-1 polypeptide or a nucleic acid encoding a Bif-1 polypeptide. Efficacy can also be measured by failure of an individual to worsen as assessed by stabilization of the neurological disease, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the neurological disease; or (2) relieving the disease, e.g., causing regression of symptoms; and (3) preventing or reducing the likelihood of the development of the neurological disease, or preventing secondary issues associated with the neurological disease (e.g., injury, accidents, etc.).

An effective amount for the treatment of a disease means that amount which, when administered to a mammal in need thereof, is sufficient to result in effective treatment as that term is defined herein, for that disease. Efficacy of an agent can be determined by assessing physical indicators of the neurological disease, such as e.g., improved cognitive function and/or memory, reduced amyloid beta protein accumulation, reduced neuronal cell death, improved neuronal cell function etc.

Clinically, an effective dose of an agent that increases Bif-1 activity and/or expression as described herein, or effective regimen, is a combination of dose and dosing that provides for an improvement in the symptoms associated with the particular neuronal or neurodegenerative disease, a non-limiting example of which is Parkinson's disease. Parkinson's disease can be clinically assessed by the United Parkinson's Disease Rating Scale (UPDRS), or the use of surrogate markers. For example, the motor abilities of a Parkinson's patient may improve, where motor symptoms may include motor fluctuations, dyskinesias, off-period dystonia, freezing, and falls. Alternatively, improvement can be assessed by imaging, e.g. by monitoring of dopamine uptake, or striatal neuron function. The standard tool for tracking Parkinson's disease progress and response to therapy is the United Parkinson's Disease Rating Scale (UPDRS). The UPDRS is subdivided into three scales including cognitive and mood aspects, motor aspects, and activities of daily living (ADL). A lower score indicates a better condition than a higher score. The UPDRS is readily available, e.g. see Fahn S, Elton R, Members of the UPDRS Development Committee. In: Fahn S, Marsden C D, Caine D B, Goldstein M, eds. Recent Developments in Parkinson's Disease, Vol 2. Florham Park, N.J. Macmillan Health Care Information 1987, pp 15 3-163, 293-304.

Further clinical tests for assessing neuroprotection can be used in the clinical setting by those of skill in the art of medicine. The treatment of a neurodegeneration as a result of brain injury can be monitored by employing a variety of neurological measurements. For example, a therapeutic response can be monitored by determining if, for example, there is an improvement in the subjects a) maximum daily Glasgow Coma Score; b) duration of coma; 3) daily intracranial pressure (ICP)—therapeutic intensity levels; 4) extent of cerebral edema/mass effect measured on serial CT scans; and, 5) duration of ventilator support. A brief description of each of these measurements is provided below.

The Glasgow Coma Score (index GCS) is a reflection of the depth of impaired consciousness and is best obtained following initial resuscitation (oxygenation, rehydration and support of blood pressure) but prior to use of sedating drugs, neuromuscular blocking agents, or intubation.

The ICP of patients with severe brain injury is often monitored with an intracranial pressure device. Monitoring ICP can provide a measure of cerebral edema. However, inherent variability and analysis complexities due to therapeutic intervention exist. To adjust for these interventions a therapeutic intensity scale was developed. This scale, known as the Therapeutic Intensity Level (TIL), measures treatment aggressiveness for elevated ICPs (Allolio et al. (1995) European Journal of Endocrinology 133(6): 696-700; Adashi et al. (1996) Reproductive endocrinology, surgery, and technology Philadelphia: Lippincott-Raven; and, Beers et al. eds. (1999) The Merck Manual of Diagnosis and Therapy. 17th ed., Merck Sharp & Dohme Research Laboratories, Rahway, N.J.).

The extent of cerebral edema and mass effect can be determined by CT scans. For example, the volume of focal lesions can be measured. Mass lesions, either high-density or mixed-density abnormalities, are evaluated by measuring the area of the abnormality as a region of interest, multiplying the area by the slice thickness, and summing these volumes for contiguous slices showing the same lesion. Each lesion is measured three times, and the mean volume entered. This technique has been shown to be reliable (Garcia-Estrada et al. (1993) Brain Res 628(1-2): 271-8). Intracerebral lesions can be further characterized by location (frontal, temporal, parietal, occipital, basal ganglia, or any combination).

The Functional Independence Measure (FIM) can be used to assess physical and cognitive disability. It contains 18 items in the following domains: self-care, sphincter control, mobility, locomotion, communication, and social cognition (Baulieu (1997) Mult Scler 3(2):105-12). The FIM has demonstrated reliability and validity as an outcome measure following moderate and severe TBI (Jung-Testas et al. (1994) J Steroid Biochem Mol Biol 48(1):145-54).

The Sickness Impact Profile is one method for measuring self-perceived health status (Schumacher et al. (1995) Ciba Found Symp 191: p. 90-112 and Koenig et al. (1995) Science 268(5216):1500-3). It consists of 136 questions divided into 12 categories: sleep and rest, eating, work, home management, recreation and pastimes, ambulation, mobility, body care and movement, social interaction, alertness, behavior, emotional behavior, and communication. It has been widely used across a variety of diseases and injuries, including head injury (Thomas et al. (1999) Spine 24:2134-8). Baseline SIP scores will reflect pre-injury health status, while follow-up scores will examine post-injury functioning.

### Screening Assays

Screening assays as contemplated herein can be used to identify modulators, i.e., candidate or test compounds or agents (e.g., peptides, antibodies, peptidomimetics, small molecules (organic or inorganic) or other drugs) which modulate Bif-1 expression and/or activity.

The term “candidate agent” is used herein to mean any agent that is being examined for ability to modulate the activity or expression of Bif-1. Although the method generally is used as a screening assay to identify previously unknown molecules that can act as a therapeutic agent, the screening described herein can also be used to confirm that an agent known to have such activity, in fact has the activity, for example, in standardizing the activity of the therapeutic agent. A candidate agent can be any type of molecule, including, for example, a peptide, a peptidomimetic, a polynucleotide, or a small organic molecule, that one wishes to examine for the ability to modulate a desired activity, such as, for example, increasing Bif-1 expression and/or activity (e.g., neuroprotective activity). It will be recognized that the methods described herein are readily adaptable to a high throughput format and, therefore, the methods are convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. Nos. 5,622,699; 5,206,347; Scott and Smith, Science 249:386-390, 1992; Markland et al., Gene 109:1319, 1991; each of which is incorporated herein by reference in their entireties); a peptide library (U.S. Pat. No. 5,264,563, which is incorporated herein by reference); a peptidomimetic library (Blondelle et al., Trends Anal. Chem. 14:8392, 1995; a nucleic acid library (O'Connell et al., supra, 1996; Tuerk and Gold, supra, 1990; Gold et al., slpra, 1995; each of which is incorporated herein by reference in their entireties); an oligosaccharide library (York et al., Carb. Res., 285:99128, 1996; Liang et al., Science, 274:1520-1522, 1996; Ding et al., Adv. Expt. Med. Biol., 376:261-269, 1995; each of which is incorporated herein by reference in their entireties); a lipoprotein library (de Kruif et al., FEBS Lett., 399:232-236, 1996, which is incorporated herein by reference in their entireties); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference in their entireties).

Accordingly, the term “agent” as used herein in the context of screening means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any Bif-1 protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof.

In certain embodiments, the candidate agent is a small molecule having a chemical moiety. Such chemical moieties can include, for example, unsubstituted or substituted alkyl, aromatic, or heterocyclyl moieties and typically include at least an amine, carbonyl, hydroxyl or carboxyl group, frequently at least two of the functional chemical groups, including macrolides, leptomycins and related natural products or analogues thereof. Candidate agents can be known to have a desired activity and/or property, or can be selected from a library of diverse compounds. Also included as candidate agents are pharmacologically active drugs, genetically active molecules, etc. Such candidate agents of interest include, for example, chemotherapeutic agents, hormones or hormone antagonists, growth factors or recombinant growth factors and fragments and variants thereof Exemplary of pharmaceutical agents suitable for use with the screening methods described herein are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all of which are incorporated herein by reference in their entireties. Also included are toxins, and biological and chemical warfare agents, for example see Somani, S. M. (Ed.), “Chemical Warfare Agents,” Academic Press, New York, 1992), the contents of which is herein incorporated in its entirety by reference. Candidate agents, such as chemical compounds, can be obtained from a wide variety of sources including libraries of synthetic or natural compounds, such as small molecule compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Synthetic chemistry transformations and protecting group methodologies (protection and deprotection) useful in synthesizing the candidate compounds for use in the screening methods described herein are known in the art and include, for example, those such as described in R. Larock (1989) Comprehensive Organic Transformations, VCH Publishers; T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof, the contents of each of which are herein incorporated in their entireties by reference. Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al (1994) Angew. Chem. Int. Ed. Engl. 33:2061; and Gallop et al. (1994) J. Med. Chem. 37:1233, the contents of each of which are herein incorporated in their entireties by reference. Libraries of candidate agents can also, in some embodiments, be presented in solution (e.g., Houghten (1992), Biotechniques 13:412-421), or on beads (Lam (1991), Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.), the contents of each of which are herein incorporated in their entireties by reference. The test compounds or candidate agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.). The methods described herein further pertain to novel agents identified by the above-described screening assays. With regard to intervention, any treatments which modulate Bif-1 expression and/or activity (e.g., Bif-1 mediated neuroprotective activity) should be considered as candidates for human therapeutic intervention.

In one embodiment, a screening assay is a cell-based assay comprising contacting a cell (e.g., neuron or neurite) in culture with a candidate agent and determining the ability of the candidate agent to modulate (e.g., induce or inhibit) Bif-1 activity and/or expression (e.g., neuron-specific Bif-1 activity and/or expression).

The screening assays described herein can be used alone, or in combination with at least one other screening assay as described herein.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Example 1

### Bif-1 and Regulation of Apoptosis and Mitochondrial Morphology

In the present study, the inventors report that Bif-1 exhibits neuron-specific functions which are opposite to what has been described for non-neuronal cells, in the regulation of apoptosis and mitochondrial morphology. While Bif-1 knockdown in fibroblasts attenuated DNA damage-induced apoptosis and promoted mitochondrial elongation, Bif-1 knockdown in primary mouse cortical neurons increased their sensitivity to p53-dependent apoptosis and promoted mitochondrial fragmentation. Restoration of Bif-1 expression in Bif-1 deficient neurons corrected the mitochondrial phenotype in an isoform-specific manner. The unique Bif-1 functions observed in neurons may be explained by longer, alternatively spliced forms of Bif-1 previously identified as brain specific (13), which the inventors have now demonstrated are neuron specific. Mice lacking Bif-1 were also more sensitive to ischemic damage induced by middle cerebral artery occlusion. These findings indicate that Bif-1 promotes mitochondrial elongation and enhances cell viability in neurons, in contrast to other cell types where it acts as a mitochondrial fragmentation-promoting, pro-apoptotic protein. As such, modulation of the expression and/or activity of Bif-1 can be used as novel cell-specific modifiers of neuronal degeneration.

### Bif-1 Enhances Neuronal Viability

Neuronal apoptosis mediated by p53 is Bax-dependent (14) and involves changes in mitochondrial dynamics (elongation) that are not seen in non-neuronal cells (15, 16). Bax promotes mitochondrial apoptosis and also regulates mitochondrial dynamics (14, 17-19), and Bif-1 has been shown to bind and activate Bax upon apoptosis induction in non-neuronal cells while also promoting mitochondrial fragmentation (2, 3). Here, the inventors sought to determine if Bif-1 also plays a role in mediating a p53-Bax apoptotic pathway in neurons.

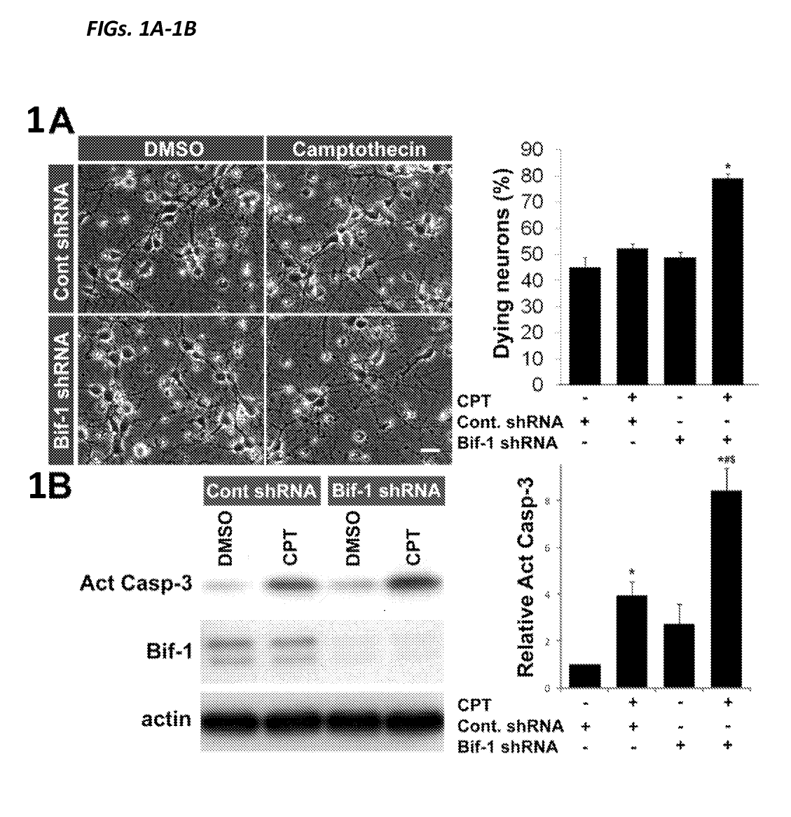

Camptothecin (CPT) is a topoisomerase I inhibitor that causes DNA damage and activates p53 to induce apoptosis in a number of cell types, including neurons (14-16, 20). Treatment with CPT for 12 hr increased morphologically discernible cell death only minimally over DMSO control (FIG. 1A), but did promote a significant increase in caspase-3 activation, indicating that neurons were undergoing apoptosis (FIG. 1B). Under these conditions, however, Bif-1 protein levels remained unchanged in CPT-treated neurons (FIG. 1B blot: 96.6%±7.1% relative to control treated, n=10), contrary to previous studies with non-neuronal cells showing that Bif-1 levels increase during apoptotic conditions (21). shRNA-mediated knockdown was employed to assess what role Bif-1 plays in these neurons undergoing apoptosis. Bif-1 knockdown alone did not immediately cause significant changes in neuronal viability (3.5 days after infection), and elevated activated caspase-3 levels only slightly. Moderate neuron loss was eventually observed with an extended duration (5 days) of infection relative to control shRNA (not shown). A constitutive pro-survival role for Bif-1 became evident when knockdown neurons (3 days after infection) were challenged with CPT. In these knockdown neurons, CPT treatment resulted in not only a further increase in caspase-3 activation (FIG. 1B) but also a significant induction of cell death at 12 hr of treatment, when there was only a small increase in cell death in control shRNA virus-infected neurons (FIG. 1A).

### Bif-1 Promotes Mitochondrial Elongation in Neurons

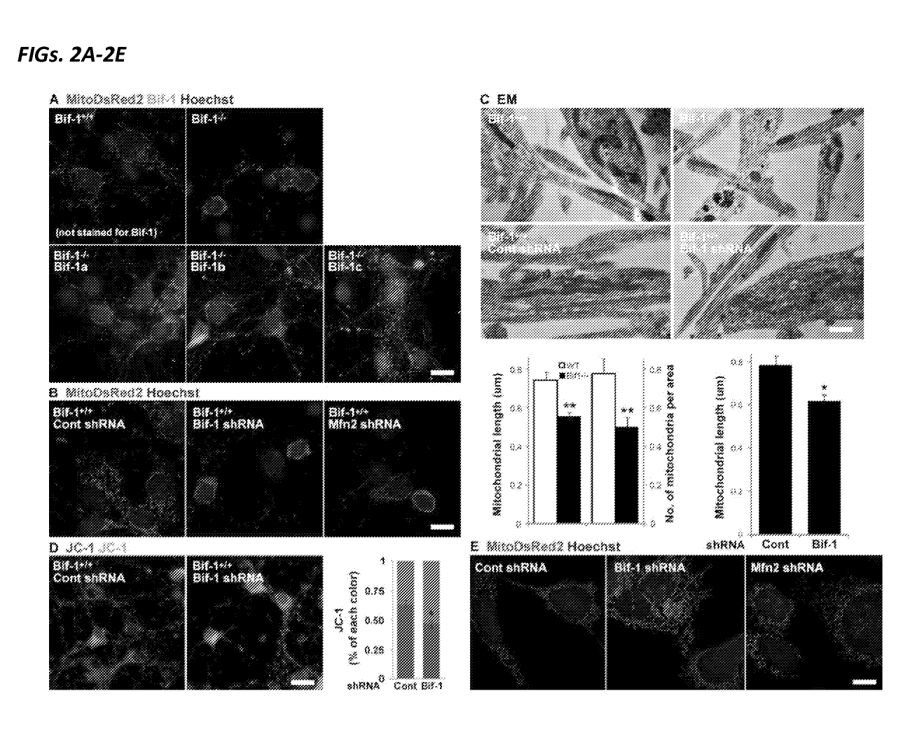

Since Bif-1 exerted a different effect on apoptosis in neurons compared to non-neuronal cell types, the inventors next investigated the influence of Bif-1 on mitochondrial morphology in neurons. Neurons cultured from Bif-1^{-/-} mice displayed greatly reduced numbers of mitochondria in neurites compared to neurons from Bif-1^{+/+} mice, while the mitochondria in the cell bodies appeared smaller and more punctate in Bif-1^{-/-} neurons, as visualized by MitoDsRed2 fluorescence (FIG. 2A).

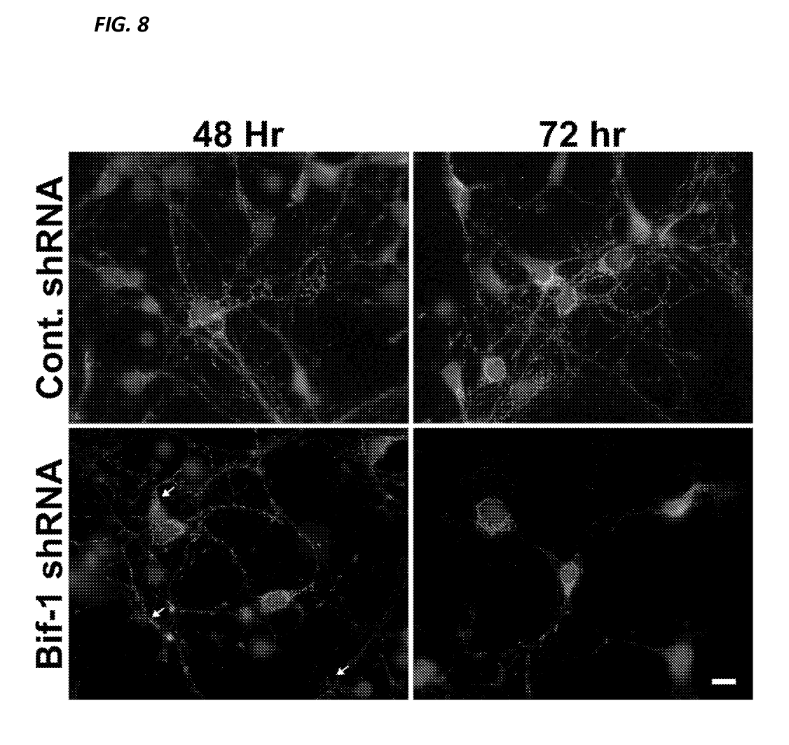

Bif-1 knockdown in wild-type neurons was then used to validate the observations seen in Bif-1^{-/-} neurons. Bif-1 shRNA-infected neurons displayed highly fragmented mitochondria that were almost exclusively perinuclear (FIG. 2B), similar to the phenotype observed with mitofusin-2 (Mfn2) knockdown which is known to promote mitochondrial fission in neurons (22, 23). An intermediate phenotype displaying partial fragmentation was observed at an earlier time point (2 days) when mitochondria are still present in neurites (arrows in FIG. 8). These changes, based on MitoDsRed2 fluorescence, were also validated (not shown) by immunostaining with the outer mitochondrial membrane marker, Tom20 (24). Electron microscopy (EM) analysis of mitochondrial size in neurites revealed smaller mitochondria in both Bif-1 deficient and Bif-1 depleted neurons, and confirmed that there were fewer mitochondria present in Bif-1 deficient neurons (FIG. 2C).

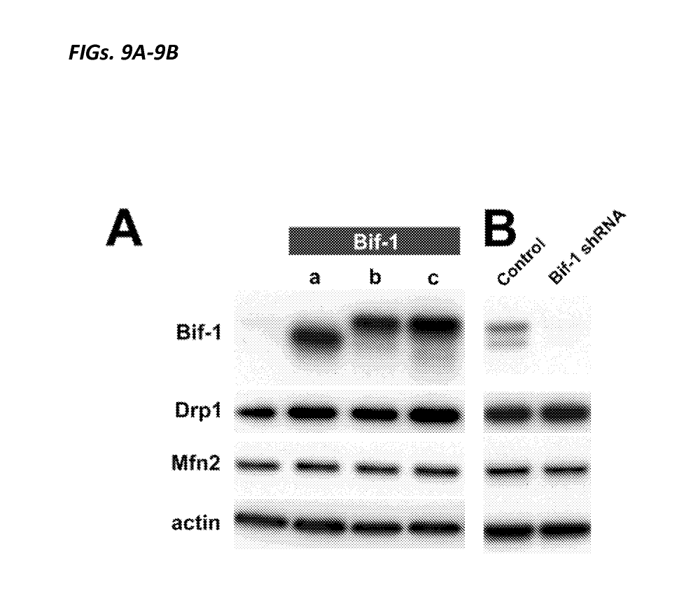

Accumulation of smaller mitochondria is evident in a histogram of mitochondrial size in Bif-1 deficient neurons where there is a 2.5 fold increase in the presence of mitochondria less than 3 microns in length. Bif-1 knockdown in neurons also induced mitochondrial depolarization, as visualized by a shift in JC-1 staining (FIG. 2D), indicating that loss of Bif-1 in neurons compromises mitochondrial bioenergetic function. The effects of Bif-1 knockdown on mitochondrial morphology in neurons do not appear to be mediated by alterations in other major mitochondrial fission/fusion proteins responsible for regulation of neuronal mitochondrial length (22), as the inventors did not observe any change in Drp1 or Mfn2 (FIG. 9).

In contrast, Bif-1 knockdown in mouse embryonic fibroblasts resulted in more elongated, interconnected mitochondria (FIG. 2E), consistent with previous results in non-neuronal cells (3, 25), while Mfn2 knockdown again resulted in smaller, fragmented mitochondria (FIG. 2E). Thus, although Mfn2 behaves as a fusion protein both in neurons and fibroblasts, Bif-1 exerts disparate effects on mitochondrial morphology between neurons and non-neuronal cells.

### Neurons Specifically Express Longer, Alternatively Spliced Isoforms of Bif-1

The contrasting effects of Bif-1 knockdown between neurons and non-neuronal cells may be attributable to the expression of unique isoforms in neurons, which was recently demonstrated for Drp1 (22). It has been previously reported that different Bif-1 isoforms exist in brain (13).

Using RT-PCR (FIG. 3A) and Western blot analysis (FIG. 3B), the inventors demonstrated that only neurons and neuroblastoma cells expressed longer isoforms of Bif-1 message and protein. In contrast, all other cell types tested, including fibroblasts, astrocytes and neural progenitor cells, expressed only the shortest isoform, Bif-1a (FIG. 3). While the mRNA sequences for the various isoforms were resolvable (FIG. 3A), protein blots (FIG. 3B) were only able to differentiate between neuron-specific isoforms showing up as a single band (Bif-1b/c) and the ubiquitous isoform (Bif-1a). However, the relative mRNA levels of the Bif-1 isoforms indicate that in both mouse primary neurons (FIG. 3A) and in mouse and human brain tissue samples (not shown), Bif-1b is preferentially expressed over the other neuron-specific forms. Thus, although the larger protein band found in neurons (Bif-1b/c, FIG. 3B) is likely to be predominantly Bif-1b the term “Bif-1b/c” is used throughout this study.

DNA sequence analysis of these differentially spliced isoforms confirmed that the splicing events in neurons involve inclusion/exclusion of exons 6 and 7, located within the N-BAR domain, which is responsible for the membrane binding and curvature function of Bif-1 (13). A diagram depicting the different Bif-1 isoforms is shown in FIG. 3C. The ubiquitously expressed Bif-1a is the shortest, lacking exons 6 and 7, while Bif-1c is the longest, containing both exons. Bif-1b differs from Bif-1c in that it contains a short (39 bp) form of exon 6 (6S) rather than the long (89 bp) form (6L). Curiously, while Bif-1a and Bif-1b were found in multiple sequence databases, Bif-1c was absent; instead, a different isoform (NM_{—}001206651.1 transcript variant 2, which was designate Bif-1d) was found, which contains full length exon 6 but lacks exon 7. The inventors were unable to detect the expression of Bif-1d mRNA, however, in either mouse neurons or whole mouse and human cortical brain homogenate using PCR primers that would specifically amplify this isoform (not shown). Neurons and neuroblastoma cells expressed an additional isoform that contains exon 7 but lacks exon 6; a search of the NCBI database revealed one mRNA transcript that contains this sequence (XM_{—}004752659.1, which we designate Bif-1e). Without wishing to be bound by theory, the presence of neuron-specific isoforms that involve alternative usage of exons 6 and 7 in the N-BAR domain indicate that neuron-specific functions of Bif-1 may involve its modified ability to bind and manipulate membranes.

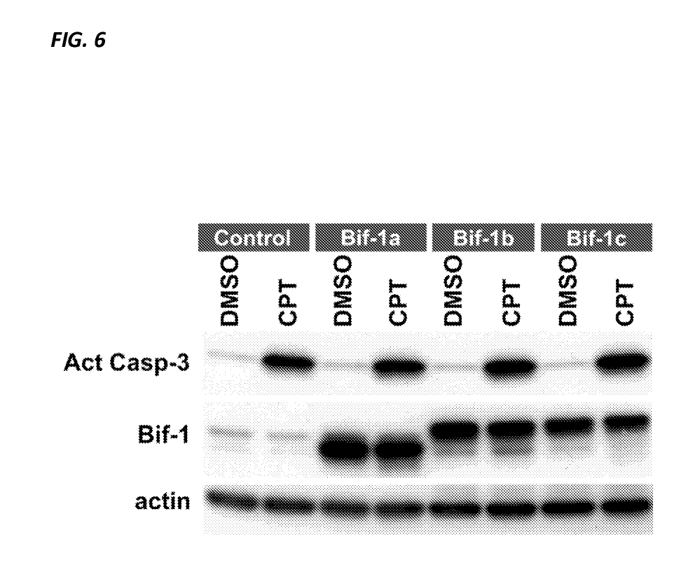

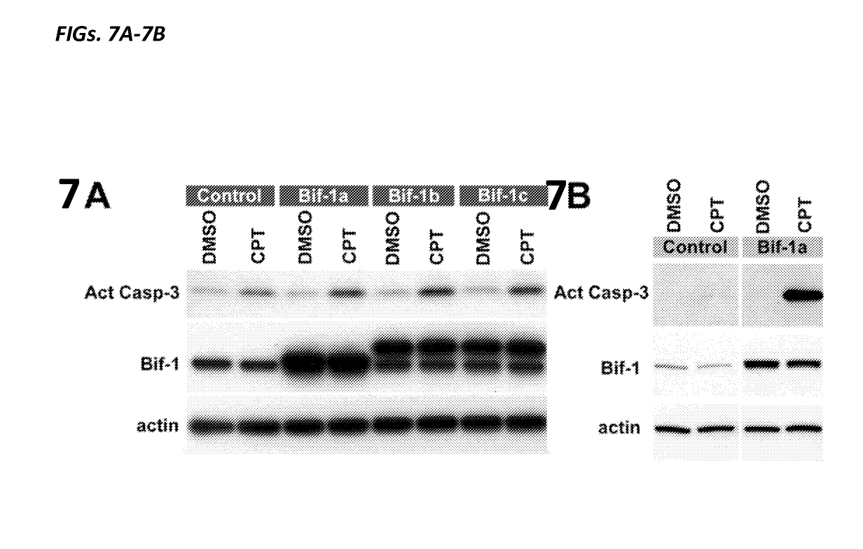

Overexpression of different human Bif-1 isoforms had no effect on CPT-induced caspase-3 activation (FIG. 6) or cell death (not shown). In contrast, Bif-1 overexpression in both mouse embryonic fibroblasts and NIH3T3-fibroblasts augmented CPT-induced apoptosis (FIG. 7), consistent with previous reports of Bif-1 being a pro-apoptotic protein in non-neuronal cells (2). In Bif-1^{-/-} neurons (FIG. 2A), and in neurons treated with Bif-1 shRNA (not shown), overexpression of Bif-1 isoforms partially reversed the fragmented mitochondrial phenotype, with the neuron-specific isoforms Bif-1b and Bif-1c having a more pronounced effect than isoform Bif-1a. The intracellular distribution of overexpressed isoform Bif-1c protein appeared more punctate than the other two isoforms, which only exhibited a diffuse distribution in both the cell body and neurites (FIG. 2A). However, it was rare to observe specific co-localization of any Bif-1 isoform with mitochondrial markers (MitoDsRed2 or Tom20) in neurons.

### Bif-1^{-/-} Mice are More Vulnerable to Ischemic Injury

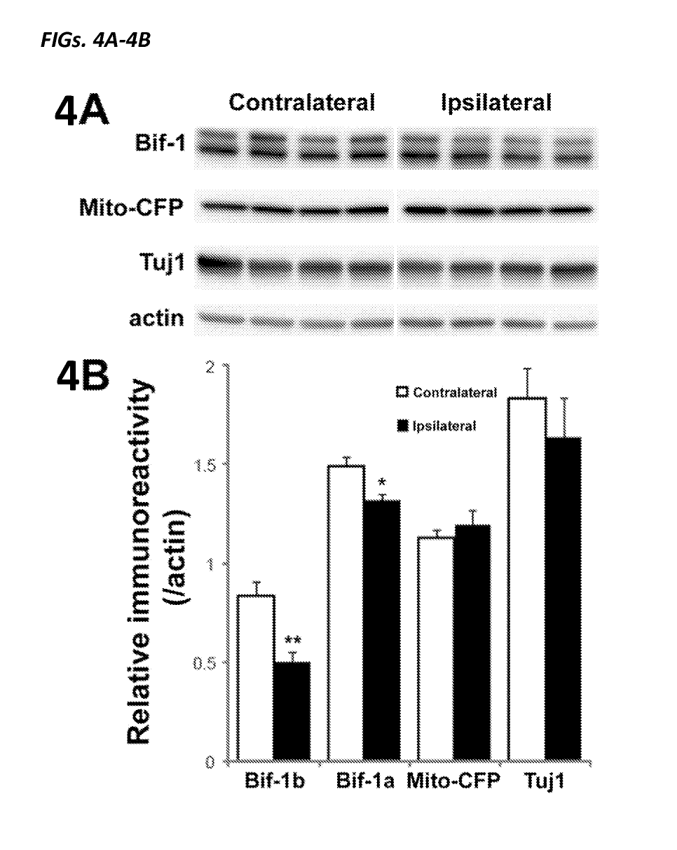

Based upon their in vitro findings the inventors sought to determine, using an in vivo model of neuronal stress, if Bif-1 was in fact required to maintain neuronal viability. Since Bif-1 expression increases under conditions of cell death in non-neuronal cells where Bif-1 is pro-apoptotic (21), it was reasoned that Bif-1 expression is decreased in stressed neurons where Bif-1 is expected to be pro-survival. The inventors employed the middle cerebral artery occlusion (MCAO) model of ischemic injury to test this, using wild-type mice expressing mitochondrial-targeted CFP (Mito-CFP) under the neuron-specific Thy1 promoter (26). Two days post-surgery, cortical tissue dorsal to the infarct, containing the ischemic penumbra, was dissected out and analyzed for Bif-1 protein expression, with the corresponding unaffected contralateral region used as a control. As expected, both Bif-1a and Bif-1b/c expression was decreased, with the neuron-specific isoform showing a greater reduction (FIG. 4). This was not due to neuronal cell loss as there was essentially no decrease in the neuronal specific marker Tuj1 or neuronal expressed Mito-CFP at this time point in the penumbra (FIG. 4).

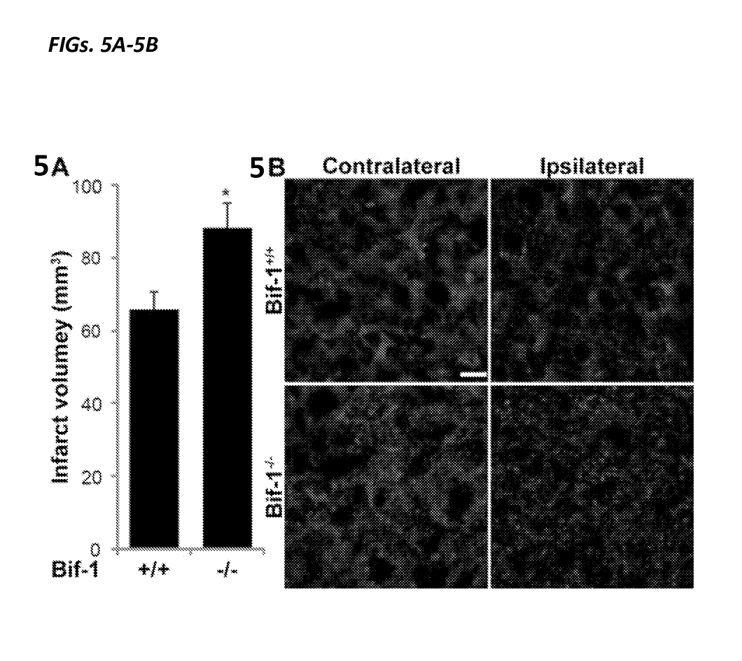

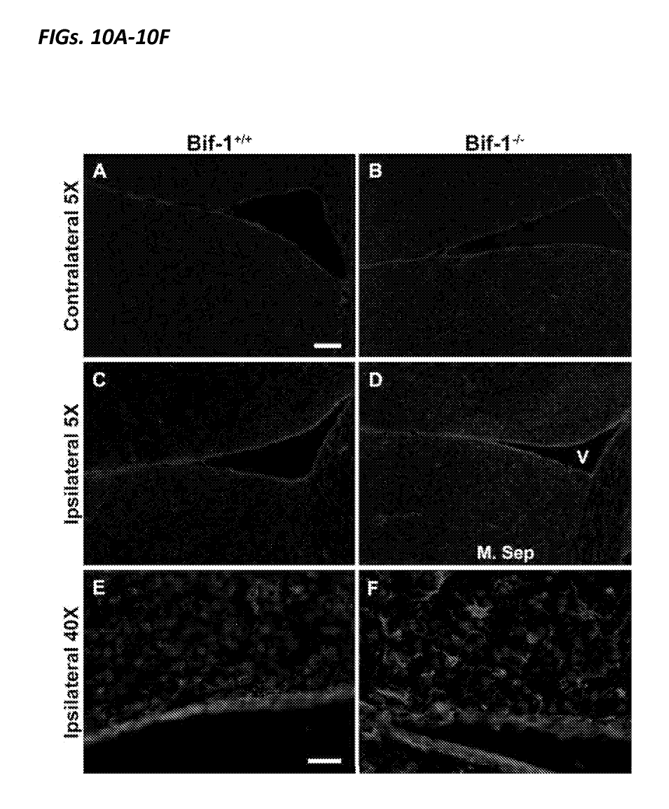

To validate the hypothesis that the loss of Bif-1 sensitizes neurons to stress-induced death, Bif-1^{+/+} and Bif-1^{-/-} mice were subjected to MCAO. At day 2 post-surgery, Bif-1^{-/-} brains displayed on average a 4.6% larger infarct volume as assessed by triphenyl tetrazolium chloride (TTC) staining (FIG. 5A-B), indicating that neurons lacking Bif-1 were more vulnerable to ischemic injury. To address MCAO-induced changes in mitochondrial morphology, the inventors used Bif-1^{+/+} and Bif-1^{-/-} mice expressing Mito-CFP to visualize “neuronal” mitochondria exclusively. Bif-1^{-/-} Mito-CFP mice expressed fainter Mito-CFP signal compared to wild-type throughout the brain. Under uninjured conditions (contralateral side), neuronal mitochondria in Bif-1^{-/-} mice appeared more fragmented in the cerebral cortex (FIG. 5C, upper panels) and striatum (not shown) compared to wild-type mice. After MCAO (ipsilateral side compared to contralateral side), mitochondria in cortical neurons of the penumbra became increasingly fragmented, with much greater fragmentation seen in Bif-1^{-/-} animals (FIG. 5C, lower panels). This correlates well with the increased volume of infarct revealed by TTC staining in Bif-1^{-/-} animals (FIGS. 4A,B) and indicates that neurons lacking Bif-1 suffer additional mitochondrial defects in response to ischemic injury, although it is uncertain if the mitochondrial fragmentation and neuronal death are causally linked. Resting astrocytes on the uninjured contralateral side, as visualized by staining for the astrocyte specific marker glial fibrillary acidic protein (GFAP), appeared similar in number and morphology between Bif-1^{-/-} and Bif-1^{+/+} animals. Astrocytes on the injured ipsilateral side, however, appeared more activated in Bif-1^{-/-} animals based on their overall larger size and number of processes (FIG. 10), consistent with the presence of more extensive underlying neuronal damage. Furthermore, the zone of reactive astrocytes around the infarct was expanded more medially in Bif-1^{-/-} animals (FIG. 10), consistent with their larger infarct volume. Taken together, these data indicate that Bif-1 is required for maintaining neuronal survival and its loss sensitizes neurons to cell death induced by stress such as ischemic injury.

Several studies have shown that Bif-1 is a pro-apoptotic protein and promotes mitochondrial fragmentation (1-3, 21). In the present study, however, it was observed that Bif-1 in neurons has the opposite functions. Knockdown of Bif-1 in mouse postnatal cortical neurons enhanced the apoptotic response to DNA damage and elicited mitochondrial fragmentation. Bif-1 knockout/knockdown neurons in culture contained fragmented mitochondria, particularly in neurites, but restored normal mitochondrial morphology upon Bif-1 overexpression. In a mouse model of stroke, Bif-1 expression was decreased in the penumbra and the stroke outcome was exacerbated in Bif-1^{-/-} animals, manifesting larger infarct volumes, more fragmented mitochondria and a more extensive pattern of astrocytic activation. These findings indicate that, in neurons, Bif-1 has pro-survival functions and promotes mitochondrial elongation. Finally, the inventors showed that neurons express unique, longer isoforms of Bif-1, which may partly explain these distinct Bif-1 functions in neuronal and non-neuronal cells.

### Bif-1 Functions as a Pro-Survival Factor in Neurons

The mechanism by which Bif-1 promotes viability in neurons has not been elucidated, but the pro-apoptotic function in non-neuronal cells is thought to involve its binding to and activation of Bax and Bak (1, 2). Induction of apoptosis leads to Bif-1 association with Bax on mitochondria and a conformational change in Bax (1, 2), as well as an increase in Bif-1 expression (21). Additionally, overexpression of Bif-1 during apoptosis promotes a Bax conformational change and enhances apoptosis (1), whereas knockdown of Bif-1 prevents the activation of both Bax and Bak (2). In non-neuronal cells, p53 can directly bind and activate Bax to trigger apoptosis (27), whereas in neurons, Bax activation may be restricted through p53 transcriptional activation of PUMA (15). In addition, neurons do not express full length Bak, but rather an alternatively spliced, BH3 domain-only form of Bak (28), which may not be subject to regulation by Bif-1. Without wishing to be bound by theory, these key differences in the apoptotic pathways mediated by Bax/Bak in neurons may explain, in part, the opposing actions Bif-1 exerts on cell death and survival between non-neuronal and neuronal cells.

There have been few studies on Bif-1 in neurons. In PC-12 cells, Bif-1 has been shown to be involved in nerve growth factor receptor trafficking, a key regulatory event in neuronal survival and differentiation (29). Bif-1 knockdown resulted in TrkA receptor degradation, attenuation of Erk signaling, and inhibition of neurite outgrowth (29). Without wishing to be bound by theory, it is possible that knockdown of Bif-1 could prevent neurons from receiving pro-survival signals, resulting in enhanced sensitivity to injury. However, the same group demonstrated that Bif-1 knockdown attenuated 1-methyl-4-phenylpyridinium (MPP⁺) and mutant alpha-synuclein-mediated cell death in cultured cortical neurons (30). Depending on the neurotoxic insult, Bif-1 levels or activity can either decrease (such as with stroke; FIG. 4) or increase (30); normalization of Bif-1 function in either direction could then be neuroprotective. Without wishing to be bound by theory, this may explain why overexpression of Bif-1 did not protect against CPT-induced apoptosis in neurons (Supplemental FIG. 1), since CPT did not induce a decrease in Bif-1 (FIG. 1). There have been recent studies demonstrating that Bif-1 levels can either increase or decrease depending on the type of cancer (31, 32), supporting the idea that perturbations in Bif-1 levels or activity in either direction can lead to alterations in cell viability and function. Another possibility relates to the mechanisms of cell death; in Parkinson's disease, abnormally increased autophagy is thought to be a causative factor in neuronal death (30), while in stroke, studies have shown autophagy to be neuroprotective (33). The inventors also examined Bif-1 expression in other neurodegenerative diseases such as Alzheimer's disease to lead to new insights regarding how Bif-1 functions to promote neuronal survival or apoptosis.

In the MCAO model of stroke, Bif-1 levels decreased in the ischemic penumbra (FIG. 4), an area where neurons can either recover or undergo apoptosis (34). The decrease was greater for neuron-specific isoforms (Bif-1b/c) than for Bif-1 a, indicating that this was mainly a neuronal event. This decrease at day 2 post-MCAO occurred with no neuronal death yet taking place, as indicated by the lack of change in the levels of the neuron-specific marker Tuj 1 or neuron-specific Mito-CFP. Along with the larger infarcts observed in Bif-1^{-/-} animals, this drop in Bif-1 expression preceding the potential neuronal death that could ensue in the penumbra points to the possibility that Bif-1 plays a pro-survival role for neurons, and decreased Bif-1 expression leads to increased vulnerability of neurons to apoptotic stress. The inventors also observed enhanced astrocytic activation in response to MCAO in Bif-1^{-/-} animals. Although this likely represents a glial response to greater neuronal damage precipitating in the absence of Bif-1, the possibility exists that Bif-1 also plays a key role in intrinsic glial responses to ischemic conditions that can either counteract or exacerbate neuronal stress. Neuron or glia-specific overexpression or knockout of Bif-1 could address this issue.

Loss of Bif-1 in Neurons Resulted in Fragmented Mitochondria

Bif-1 knockdown/knockout neurons, either in culture (FIG. 2A-B) or in vivo (FIG. 5C), displayed smaller mitochondria, most noticeably in neurites where individual mitochondria can be readily discerned. EM analysis of cultured neurons corroborated these results and further showed the presence of very small (fragmented) mitochondria. Conversely, overexpression of Bif-1 restored a normal mitochondrial phenotype in Bif-1^{-/-} neurons, but in an isoform specific manner with the neuron-specific isoforms, Bif-1b and Bif-1c, being more efficacious than the ubiquitously expressed Bif-1a. Consistent with the failed maintenance of mitochondrial morphology, mitochondrial membrane potential in Bif-1 knockdown neurons was found to be compromised. Thus, in sharp contrast to its function in non-neuronal cells, Bif-1 or, more specifically, neuron-specific Bif-1 isoforms in neurons appear to be required for maintaining the size and bioenergetic function of mitochondria. It comes as no surprise, therefore, that when put under stress (DNA damage, MCAO), Bif-1 knockout/knockdown neurons exhibit an exacerbated apoptotic outcome. These results indicate that the ability of Bif-1 to modulate mitochondrial morphology and bioenergetic capabilities has direct implications for its pro-survival action in neurons.

Interestingly, Bif-1 deficiency also resulted in fewer total mitochondria in neurites, as confirmed by EM (FIG. 2C). If Bif-1 deficiency does indeed result in increased mitochondrial fission, one would expect a larger number of small mitochondria, keeping the overall mitochondrial volume constant, but this was not the case. It is possible that smaller mitochondria resulting from Bif-1 deficiency are selectively targeted for degradation. Although it is unknown why mitochondria in Bif-1 deficient neurons become smaller, it appears that Bif-1 does not influence mitochondrial fusion rates directly (3).

Depletion of Bif-1, both in culture and in vivo resulted, in smaller, fragmented mitochondria and increased sensitivity to neurotoxic stress. While loss of Bif-1 in culture or in vivo resulted in abnormal mitochondrial morphology, without any immediate loss of neuronal viability, neurons exhibited a marked increase in apoptosis (activated caspase-3) or death (TTC stain) upon addition of a stress in the form of CPT or MCAO. This indicates that the mitochondrial dysfunction caused by Bif-1 deficiency resulted in heightened sensitivity to stress. The data show that Bif-1 acts as a pro-survival factor in neurons and is not the pro-apoptotic protein previously reported. Early detection of lowered Bif-1 expression and subsequent restoration may prove useful in mitigating neuronal dysfunction and death resulting from nervous system injury and disease.

### Materials and Methods

### Animals

C57BL/6 mice were obtained from the Jackson laboratory (Bar Harbor, Me.). Bif-1 deficient (Bif-1^{+/-}) mice (35), which had been backcrossed to C57BL/6 16 times, were obtained from Dr. Hong-Gang Wang (Penn State College of Medicine). Bif-1^{+/+} and Bif-1^{-/-} mouse lines were derived and maintained by homozygous mating for less than 2 years for the present study. Mito-CFP mouse line C, which expresses mitochondrial-targeted CFP in neurons under the control of a modified Thy1 promoter (26), was obtained from the Jackson laboratory and backcrossed to C57BL/6 11 times before deriving mito-CFP homozygous animals. Bif-1^{+/+}/mito-CFP^{+/-} mice were obtained by crossing Bif-1^{+/+} and mito-CFP^{+/+} animals, while Bif-1^{-/-}/mito-CFP^{+/-} mice were obtained by crossing Bif-1^{-/-} and Bif-1^{-/-}/mito-CFP^{+/+} animals. All the genotypes were confirmed by PCR using primer sets: TGCCTCAGATGACCACCAGCCACC and TCACCACTGGGTGGAGCCGCT or CTTAGTGAGCTGTCAGGAGAGC and AGGTTCTCATGGGAACAGCGAC for wild-type and CTTAGTGAGCTGTCAGGAGAGC and TCGCCTTCTTGACGAGTTCT for knockout. Experiments with these animals were approved by the University of Washington institutional animal care committee.

### Cell Culture

Primary cultures of postnatal cortical neurons from newborn mice (P0) were prepared as previously described (20). Neurons were maintained in Neurobasal-A (Gibco, Grand Island, N.Y.) supplemented with B-27 (Gibco) and GlutaMAX-I (Gibco). Mouse embryonic fibroblasts (MEF) and NIH3T3 fibroblasts were prepared and/or maintained as previously described (15, 22, 28). When necessary, cultures were first infected with lentivirus at 10 MOI (including MitoDsRed2 for mitochondrial labeling as single or double infection) one day after plating (neurons) or splitting (fibroblasts). Four days after plating/splitting, cells were either harvested/fixed without treatment or treated with camptothecin (5 μM, Sigma, St. Louis, Mo.) for 12 hr before harvesting/fixing, unless otherwise specified. Overall neuronal viability was monitored morphologically, based on phase-contrast microscopy and nuclear morphology (Hoechst 33258 staining), as previously described (14). Other cell types used for Bif-1 expression studies included primary astrocytes, spinal cord neural progenitor cells, and human SH-SY5Y neuroblastoma cells, prepared and/or maintained as previously described (15, 22, 28).

Neuro-2a mouse neuroblastoma cells were obtained from Dr. Sen-itiroh Hakomori (Pacific Northwest Diabetes Research Institute, Seattle, Wash.) and cultured in Eagle's minimal essential medium with 10% fetal bovine serum. Cells were harvested from exponentially growing cultures.

### Plasmid Construction and Lentivirus Production

Production of DNA constructs and lentivirus for EGFP, mitochondrial-targeted DsRed2 (MitoDsRed2), control shRNA, and Mfn2 shRNA has been described (22). Bif-1 shRNA plasmids were obtained from Sigma and one sequence (CCGGCCTACTTAGAACTTCTCAATTCTCGAGAATTGAGAAGTTCTAAGTAGGTTTTT G) validated for efficient Bif-1 knockdown was packaged into lentivirus co-expressing IRES-driven EGFP as an infection marker. The cDNA encoding human Bif-1a (NM_{—}016009.4/NP_{—}057093.1) was obtained from INVITROGEN (Grand Island, N.Y.) and cloned into pCMV-Tag2B (STRATAGENE, Santa Clara, Calif.) using PCR. The nucleotide sequences unique to human Bif-1b and Bif-1c (13) were introduced into the vector using long-primer PCR. All isoforms were FLAG-tagged at the C-terminus. Plasmids were packaged into lentiviral vectors as previously described (22). All of the PCR cloned sequences in these vectors were confirmed by DNA sequence analysis.

### RT-PCR for Bif-1 Isoforms

RNA was isolated from brain homogenate or cell culture lysate, using an RNeasy isolation kit (Qiagen), and reverse transcribed using SuperScript™ II Reverse Transcriptase according to the manufacturer's instructions (INVITROGEN). The cDNAs were subjected to PCR using custom murine (5′-attcaaacatcagccttaaatttcc-3′ & 5′-aagtcatctgggcttctacaaagt-3′) or human (5′-attcaaacgtcagccttaaattttc-3′ & 5′-aagtcatctgggcttctacaaagt-3′) primers designed to amplify the region encompassing exons 6 and 7. Amplification of human and mouse cDNAs with these primers yielded 304, 367, and 415 by products from the Bif1a, Bif1b, and Bif1c isoforms, respectively. The cDNAs were subsequently cloned into pBluescript for sequencing.

### Immunoblotting and Immunofluorescence

Protein extracts for Western blot analysis were prepared as described previously (28). Primary antibodies used were mouse monoclonal Bif-1 (clone 30A882.1.1, 1:500; Imgenex, San Diego, Calif.), mouse monoclonal β-actin (clone AC-15, 1:10,000; Sigma), rabbit polyclonal activated caspase-3 (#9661, 1:1000; Cell Signaling, Danvers, Mass.), mouse monoclonal Drp1 (clone 8/DLP1, 1:1000; BD Biosciences, San Jose, Calif.), rabbit monoclonal Mfn2 (clone NIAR164, 1:2000; Epitomics, Burlingame, Calif.). Horseradish peroxidase-conjugated secondary antibodies (1:2000) were from GE Healthcare (Pittsburgh, Pa.). For quantification, images were scanned and measured for pixel intensity using NIH ImageJ software, and normalized against β-actin values.

For immunofluorescence, cells were cultured on Thermanox plastic coverslips (NALGE NUNC INTERNATIONAL, Rochester, N.Y.), fixed, permeabilized, and processed for immunostaining as described previously (20). Fluorescent microscopic images were captured on an Axiovert 200 inverted microscope (Carl Zeiss Microimaging, Thornwood, N.Y.) equipped with a cooled CCD camera (SensiCam, Cooke Corp, Auburn Hills, Mich.). Primary antibodies used were mouse monoclonal Bif-1 (clone 30A882.1.1, 1:500; Imgenex) and chicken Tuj l (1:500; Ayes, Tigard, Oreg.). The neuronal identity of cells was confirmed by immunostaining for the neuronal marker Tuj1 (not shown). Nuclei were labeled with Hoechst 33258 (2.5 μg/ml). Alexa Fluor dye-conjugated secondary antibodies (1:400) were from INVITROGEN. Fluorescent images of MitoDsRed2 were deconvolved using SLIDEBOOK (INTELLIGENT IMAGING INNOVATIONS, Denver, Colo.) and exported out with the y factor set at 0.7 for better visualization of mitochondrial morphology.

The JC-1 dye (final concentration 1 μM; Invitrogen), diluted into Ca²⁺, Mg²⁺-containing Hanks' balanced salt solution (HBSS(+); Gibco) from 1 mM stock in DMSO, was applied to live neurons for 20 min at 37° C. in a CO₂ incubator. After washing with HBSS(+), fluorescence images were captured on an AXIOVERT 200 inverted microscope. With this method, mitochondrial depolarization is reported as a decrease in the red (590 nm) to green (530 nm) fluorescence intensity ratio. Five images per condition were taken at 32X magnification, and the total pixel intensity of red and green fluorescence was obtained to calculate the ratio of red vs. green.

### Middle Cerebral Artery Occlusion (MCAO) Model of Stroke

25-30 g male Bif-1^{+/+} and Bif-1^{-/-} mice (approximately 12 weeks old) were subjected to MCAO as previously described, with a 45 minute occlusion time (36). Successful occlusion and reperfusion was confirmed using a Laser Doppler Perfusion Monitor (Moor Instruments, Wilmington, Del.). Following 2 days of recovery, brains were removed and nine 1 mm-slices were made for 2,3,5-triphenyltetrazolium chloride (TTC) staining to measure infarct volume (37). From a separate cohort of animals, which express Mito-CFP, cortical tissue dorsal to the infarct was dissected out by reference to TTC-stained adjacent slices, and protein lysates were prepared by sonication in SDS buffer (2% SDS, 10% glycerol, 50 mM Tris-Cl pH 6.8). Some of these Mito-CFP-expressing animals were also perfused with 4% paraformaldehyde 2 days after surgery, and 20 μm coronal sections were obtained using a LEICA CM1850-3-1 cryostat (Buffalo Grove, Ill.) for tissue immunofluorescence. Mounted sections were processed similarly to cell culture immunofluorescence, as previously described (20). Primary antibodies used were rabbit anti-GFAP (1:200; #G2969, Sigma) and rabbit anti-GFP (cross-reacts with Mito-CFP and amplifies its signal, 1:200; #ab6556, ABCAM, Cambridge, Mass.). Immunofluorescent images of Mito-CFP were deconvolved using Slidebook (Intelligent Imaging Innovations, Denver, Colo.) and exported out with the y factor set at 0.7.

### Electron Microscopy

Cortical neurons were plated on Aclar plastic coverslips. Three days after lentiviral infection, cultures were fixed with 2.5% glutaraldehyde in 0.1M cacodylate buffer. Fixed cultures were prepared for electron microscopy as previously described (16). For mitochondrial size measurements, electron micrographs from regions containing only neurites were obtained using a Philips CM10 transmission electron microscope at 20,000× magnification. Mitochondrial length was measured by placing a tip-to-tip line across the longest axis of each mitochondrion using the straight line tool in NIH IMAGEJ. Images with less than 5 neuritic mitochondria were discarded. For the number of mitochondria per unit area, images were taken of random fields containing neurites at 10,000× magnification. The total number of neuritic mitochondria was divided by the neurite area for each random field. Both measurements (mean mitochondrial length and number of mitochondria per unit area) represent 150-200 mitochondria counted for each experimental condition.

### Statistics

Student's t-tests, and two-way ANOVA with Tukey post hoc tests, were used where applicable. Results were considered statistically significant when p≦0.05 using PRISM Software (GRAPHPAD, La Jolla, Calif.).

### REFERENCES

1. Cuddeback S M, et al. (2001) Molecular Cloning and Characterization of Bif-1: A NOVEL Src HOMOLOGY 3 DOMAIN-CONTAINING PROTEIN THAT ASSOCIATES WITH Bax. Journal of Biological Chemistry 276(23):20559-20565.
2. Takahashi Y, et al. (2005) Loss of Bif-1 Suppresses Bax/Bak Conformational Change and Mitochondrial Apoptosis. Molecular and Cellular Biology 25(21):9369-9382.
3. Karbowski M, Jeong S-Y, & Youle R J (2004) Endophilin B1 is required for the maintenance of mitochondrial morphology. The Journal of Cell Biology 166(7):10271039.
4. Schon Eric A & Przedborski S (2011) Mitochondria: The Next (Neurode)Generation. Neuron 70(6):1033-1053.
5. Karbowski M & Youle R J (2003) Dynamics of mitochondrial morphology in healthy cells and during apoptosis. Cell Death Differ 10(8):870-880.
6. Bertholet A M, et al. (2013) OPA1 loss of function affects in vitro neuronal maturation. Brain 136(5):1518-1533.
7. Kageyama Y, et al. (2012) Mitochondrial division ensures the survival of postmitotic neurons by suppressing oxidative damage. The Journal of Cell Biology 197(4):535-551.
8. Delettre C, et al. (2000) Nuclear gene OPA1, encoding a mitochondrial dynamin-related protein, is mutated in dominant optic atrophy. Nat Genet 26(2):207-210.
9. Zuchner S, et al. (2004) Mutations in the mitochondrial GTPase mitofusin 2 cause Charcot-Marie-Tooth neuropathy type 2A. Nat Genet 36(5):449-451.
10. Waterham H R, et al. (2007) A Lethal Defect of Mitochondrial and Peroxisomal Fission. New England Journal of Medicine 356(17):1736-1741.
11. Wang X, et al. (2009) Impaired Balance of Mitochondrial Fission and Fusion in Alzheimer's Disease. The Journal of Neuroscience 29(28):9090-9103.
12. Kim J, et al. (2010) Mitochondrial loss, dysfunction and altered dynamics in Huntington's disease. Human Molecular Genetics 19(20):3919-3935.
13. Modregger J, Schmidt A A, Ritter B, Huttner W B, & Plomann M (2003) Characterization of Endophilin B1b, a Brain-specific Membrane-associated Lysophosphatidic Acid Acyl Transferase with Properties Distinct from Endophilin A1. Journal of Biological Chemistry 278(6):4160-4167.
14. Xiang H, et al. (1998) Bax Involvement in p53-Mediated Neuronal Cell Death. The Journal of Neuroscience 18(4):1363-1373.
15. Uo T, Kinoshita Y, & Morrison R S (2007) Apoptotic Actions of p53 Require Transcriptional Activation of PUMA and Do Not Involve a Direct Mitochondrial/Cytoplasmic Site of Action in Postnatal Cortical Neurons. The Journal of Neuroscience 27(45):12198-12210.
16. Wang D B, et al. (2013) Declines in Drp1 and Parkin Expression Underlie DNA Damage-Induced Changes in Mitochondrial Length and Neuronal Death. The Journal of Neuroscience 33(4):1357-1365.
17. Karbowski M, et al. (2002) Spatial and temporal association of Bax with mitochondrial fission sites, Drp1, and Mfn2 during apoptosis. The Journal of Cell Biology 159(6):931938.
18. Karbowski M, et al. (2004) Quantitation of mitochondrial dynamics by photolabeling of individual organelles shows that mitochondrial fusion is blocked during the Bax activation phase of apoptosis. The Journal of Cell Biology 164(4):493-499.
19. Steckley D, et al. (2007) Puma Is a Dominant Regulator of Oxidative Stress Induced Bax Activation and Neuronal Apoptosis. The Journal of Neuroscience 27(47):12989-12999.
20. Xiang H, et al. (1996) Evidence for p53-Mediated Modulation of Neuronal Viability. The Journal of Neuroscience 16(21):6753-6765.
21. Yang J, et al. (2010) GSK-3β promotes cell survival by modulating Bif-1-dependent autophagy and cell death. Journal of Cell Science 123(6):861-870.
22. Uo T, et al. (2009) Drp1 levels constitutively regulate mitochondrial dynamics and cell survival in cortical neurons. Experimental Neurology 218(2):274-285.
23. Pham A H, Meng S, Chu Q N, & Chan D C (2012) Loss of Mfn2 results in progressive, retrograde degeneration of dopaminergic neurons in the nigrostriatal circuit. Human Molecular Genetics 21(22):4817-4826.
24. de Brito O M & Scorrano L (2008) Mitofusin 2 tethers endoplasmic reticulum to mitochondria. Nature 456(7222):605-610.
25. Takahashi Y, et al. (2013) Bif-1 haploinsufficiency promotes chromosomal instability and accelerates Myc-driven lymphomagenesis via suppression of mitophagy. Blood 121(9):1622-1632.
26. Misgeld T, Kerschensteiner M, Bareyre F M, Burgess R W, & Lichtman J W (2007)

Imaging axonal transport of mitochondria in vivo. Nat Meth 4(7):559-561.
27. Chipuk J E, et al. (2004) Direct Activation of Bax by p53 Mediates Mitochondrial Membrane Permeabilization and Apoptosis. Science 303(5660):1010-1014.
28. Uo T, Kinoshita Y, & Morrison R S (2005) Neurons Exclusively Express N-Bak, a BH3 Domain-only Bak Isoform That Promotes Neuronal Apoptosis. Journal of Biological Chemistry 280(10):9065-9073.
29. Wan J, et al. (2008) Endophilin B1 as a Novel Regulator of Nerve Growth Factor/TrkA Trafficking and Neurite Outgrowth. The Journal of Neuroscience 28(36):9002-9012.
30. Wong A S L, et al. (2011) CdkS-mediated phosphorylation of endophilin B1 is required for induced autophagy in models of Parkinson's disease. Nat Cell Biol 13(5):568-579.
31. Coppola D, et al. (2008) Down-regulation of Bax-interacting factor-1 in colorectal adenocarcinoma. Cancer 113(10):2665-2670.
32. Fan R, et al. (2012) Bif-1 is overexpressed in hepatocellular carcinoma and correlates with shortened patient survival. Oncology letters 3(4):851-854.
33. Papadakis M, et al. (2013) Tscl (hamartin) confers neuroprotection against ischemia by inducing autophagy. Nat Med 19(3):351-357.
34. del Zoppo G J, Sharp F R, Heiss W-D, & Albers G W (2011) Heterogeneity in the penumbra. J Cereb Blood Flow Metab 31(9):1836-1851.
35. Takahashi Y, et al. (2007) Bif-1 interacts with Beclin 1 through UVRAG and regulates autophagy and tumorigenesis. Nat Cell Biol 9(10):1142-1151.
36. Baltan S, Bachleda A, Morrison R, & Murphy S (2011) Expression of Histone Deacetylases in Cellular Compartments of the Mouse Brain and the Effects of Ischemia. (Translated from English) Transl. Stroke Res. 2(3):411-423 (in English).
37. Gibson C L, Bath P M W, & Murphy S P (2005) G-CSF reduces infarct volume and improves functional outcome after transient focal cerebral ischemia in mice. J Cereb Blood Flow Metab 25(4):431-439.

### Example 2

### Bif-1 in Amyloid Beta Toxicity

The neuron specific form of the Bif-1 protein (Bif-1b) is significantly reduced in extracts of cerebral cortical tissue prepared from patients with severe dementia (Braak stage V-V) compared to extracts made from age-matched control samples. There is a trend of lower Bif-1b in patient tissue samples with stage Braak III-IV dementia, but the difference from age-matched control samples is not significant. Since the Bif-1 isoform that is lost in extracts from Braak stage V-VI is only expressed in neurons we determined if the loss of this Bif-1 isoform was due to the loss of neurons in these brain samples. To rule out that possibility we performed stereological counts of MAP2⁺ neurons and determined that Bif-1b protein levels do not correlate with neuronal cell counts demonstrating that the loss of Bif-1b does not occur as a result of losing neurons and must therefore precede neuronal cell death. Bif-1b loss is also observed in symptomatic AD mice (10 months of age) but not asymptomatic mice (1.5 months of age) expressing mutant APP and PS1, a mouse model of human AD. Importantly, there is no documented neuronal loss in the mouse model based on stereological counting. Thus, loss of Bif-1b does not seem to be dependent on neuronal loss in both human samples and in a mouse model of AD. Loss of the Bif-1b protein was also observed in extracts prepared from synaptosomes purified from human parietal cortex. Although synapses are lost in patients with advanced dementia this procedure extracts any intact synapses remaining in these patient's tissue samples. Thus, the loss of the Bif-1b protein has been validated in a separate preparation of human AD tissue.

To further delineate a role for the loss of Bif-1 in the pathogenesis of AD, we crossed AD mice (APPswe/PS1dE9) with Bif-1 knockout mice to create an AD+/Bif-1−/− mouse. The AD+/Bif-1+/+ mice showed a reduction in lifespan when compared to wild-type mice (AD-/Bif-1+/+) or Bif-1 null mice (Bif-1−/−). However, AD+ mice on a null background showed a dramatic decline in viability. The reason for the decline in survival is not known but it might be related to increased seizure frequency or intensity as AD+ mice reportedly show more seizure activity than wild-type mice. The absence of Bif-1 was found to impair cognition based on results from the Morris water maze. AD+/Bif-1-null mice took longer to learn the task and did not remember where the platform was compared to wild-type mice so they spent less time in the correct zone (where the platform should have been). This was also true for Bif-1 null mice at one year of age independently of their expressing the AD phenotype. In other words, just the absence of Bif-1 reduced learning and memory in mice at one year of age indicating that Bif-1 was required for maintaining normal cognition. Importantly, Bif-1 null mice did not show evidence of motor impairment based on their performance on the rotarod or the grip strength test. Thus, the cognitive impairment seen in one year old Bif-1−/− mice or six month old AD+/Bif-1−/− mice did not reflect a general decline in health or wellbeing of the mice. We also observed that the Bif-1-null condition dramatically promotes the formation of β-amyloid plaques which is associated with astrocytic activation (GFAP expression), a sign of a neuroinflammatory glial response. This was observed in both the cerebral cortex and the hippocampus. Quantitation showed a significant increase in both the number of amyloid plaques and the size of amyloid plaques. We conclude from these human and mouse studies that:
Loss of Bif-1 eventually results in cognitive decline on its own
Loss of Bif-1 hastens AD-related cognitive decline and mortality
Loss of Bif-1 increases amyloid burden and astrogliosis in AD mice.

Accordingly, it is contemplated herein that one or more agents that increase Bif-1 expression and/or activity can be used to prevent cognitive decline, reduce amyloid burden, reduce astrogliosis, and improve cognitive functions, such as learning and memory in a subject (e.g., a human).

### Example 3

### Bif-1 Inhibition

The inventors have shown that reducing Bif-1 enhances sensitivity to stress irrespective of the stress and thus far several different forms of stress reduce Bif-1 protein expression including stroke (ischemic injury) and Abeta toxicity. Even DNA damage, which does not reduce Bif-1 levels is enhanced when the inventors first reduced Bif-1 via shRNA. The addition of rotenone to cultured neurons was used to reduce Bif-1 protein levels and induced cell death. In certain conditions (injury or disease) it is contemplated herein that Bif-1 plays a role in excessive autophagy leading to cell death. The inventors investigated the role of Bif-1 in a mouse model of Leighs syndrome wherein the mice express a mutation in NDUFS4 (made by Dr. Richard Palmiter) that impairs complex I activity. The inventors observed a 50-60% drop in Bif-1 levels in the olfactory bulb for example well before there is evidence of cell death in that structure. Thus, the use of a Bif-1 inhibitor as described herein is contemplated for use in treating neurological diseases and disorders associated with Bif-1 mediated autophagy.

1. A method of treating a neurological disease or disorder, the method comprising:
administering a therapeutically effective amount of a composition comprising a Bif-1 polypeptide or a nucleic acid encoding a Bif-1 polypeptide to a subject having a neurological disease or disorder.

2-41. (canceled)

42. The method of claim 1, wherein the Bif-1 polypeptide is a neuron-specific Bif-1 polypeptide.

43. The method of claim 1, wherein the Bif-1 polypeptide comprises Bif-1b or Bif-1c.

44. The method of claim 1, wherein the neurological disease or disorder is selected from the group consisting of: Alzheimer's disease, Parkinson's disease, dementia, multiple sclerosis, amyotrophic lateral sclerosis (ALS), blood brain barrier permeability, vascular dementia, and neurodegenerative disease.

45. The method of claim 1, wherein the neurological disease or disorder comprises ischemic injury, hemorrhage, hypoxic injury or apoptosis.

46. The method of claim 1, wherein the Bif-1 polypeptide or nucleic acid encoding a Bif-1 polypeptide is administered to the brain, the spinal cord, or to a peripheral nerve ending.

47. The method of claim 1, wherein the Bif-1 polypeptide or nucleic acid encoding a Bif-1 polypeptide is administered systemically.

48. The method of claim 1, further comprising, prior to administering said polypeptide or nucleic acid, the step of measuring the amount of Bif-1 polypeptide in a sample from said subject, wherein the Bif-1 polypeptide or nucleic acid is only administered if the measured level of Bif-1 polypeptide is reduced relative to a reference amount.

49. A method for predicting sensitivity of a subject to neurological damage, the method comprising:
(a) measuring the amount of a Bif-1 polypeptide or fragment thereof in a subject,
(b) comparing the amount of the Bif-1 polypeptide or fragment thereof to a reference value, wherein a decrease in the amount of the Bif-1 polypeptide or fragment thereof indicates that the subject has an increased sensitivity to neurological damage.

50. The method of claim 49, wherein the neurological damage is selected from the group consisting of ischemic injury, hypoxic injury and neuronal cell death.

51. The method of claim 49, wherein the amount of Bif-1 polypeptide or fragment thereof is measured in a biological sample obtained from the subject.

52. The method of claim 51, wherein the biological sample comprises cerebral spinal fluid, urine, saliva, blood, plasma, biopsy sample or a tumor sample.

53. The method of claim 49, wherein the Bif-1 polypeptide comprises Bif-1b or Bif-1c.

54. The method of claim 49, wherein the Bif-1 polypeptide comprises a neuron-specific Bif-1 polypeptide.

55. The method of claim 49, wherein the Bif-1 polypeptide or fragment thereof are detected using magnetic resonance imaging (MRI), positron emission tomography (PET), CT scan, and nuclear magnetic resonance imaging (NMR).

56. An assay comprising:
(a) administering to a subject an agent that binds to a Bif-1 polypeptide or a fragment thereof or that binds to an mRNA encoding a Bif-1 polypeptide;
(b) detecting the amount of the agent bound to the Bif-1 polypeptide or fragment thereof or to the Bif-1 mRNA;
(c) comparing the amount of bound Bif-1 polypeptide or Bif-1 mRNA to a reference value, wherein a decrease in the amount of bound Bif-1 polypeptide or mRNA compared to the reference value indicates that the subject has an increased risk of neurological damage.

57. The assay of claim 56, wherein the agent comprises a detectable moiety.

58. The assay of claim 56, wherein the Bif-1 polypeptide comprises Bif-1b or Bif-1c.

59. The assay of claim 56, wherein the Bif-1 polypeptide comprises a neuron-specific Bif-1 polypeptide.

60. The assay of claim 56, wherein the bound agent is detected using magnetic resonance imaging (MRI), positron emission tomography (PET), CT scan, and nuclear magnetic resonance imaging (NMR).


SEQUENCE LISTING (21 sequences)

SEQ ID NO: 1 - protein, 365 aa, Homo sapiens
MNIMDFNVKKLAADAGTFLSRAVQFTEEKLGQAEKTELDAHLENLLSKAECTKIWTEKIMKQTEVLLQPNPNARIEEFVYEKLDRKAPSRINNPELLGQYMIDAGTEFGPGTAYGNALIKCGETQKRIGTADRELIQTSALNFLTPLRNFIEGDYKTIAKERKLLQNKRLDLDAAKTRLKKAKAAETRNSSEQELRITQSEFDRQAEITRLLLEGISSTHAHHLRCLNDFVEAQMTYYAQCYQYMLDLQKQLGSFPSNYLSNNNQTSVTPVPSVLPNAIGSSAMASTSGLVITSPSNLSDLKECSGSRKARVLYDYDAANSTELSLLADEVITVFSVVGMDSDWLMGERGNQKGKVPITYLELLN

SEQ ID NO: 2 - protein, 386 aa, Homo sapiens
MNIMDFNVKKLAADAGTFLSRAVQFTEEKLGQAEKTELDAHLENLLSKAECTKIWTEKIMKQTEVLLQPNPNARIEEFVYEKLDRKAPSRINNPELLGQYMIDAGTEFGPGTAYGNALIKCGETQKRIGTADRELIQTSALNFLTPLRNFIEGDYKTIAKERKLLQNKRLDLDAAKTRLKKAKAAETRNSQLNSARLEGDNIMIWAEEVTKSEQELRITQSEFDRQAEITRLLLEGISSTHAHHLRCLNDFVEAQMTYYAQCYQYMLDLQKQLGSFPSNYLSNNNQTSVTPVPSVLPNAIGSSAMASTSGLVITSPSNLSDLKECSGSRKARVLYDYDAANSTELSLLADEVITVFSVVGMDSDWLMGERGNQKGKVPITYLELLN

SEQ ID NO: 3 - protein, 402 aa, Homo sapiens
MNIMDFNVKKLAADAGTFLSRAVQFTEEKLGQAEKTELDAHLENLLSKAECTKIWTEKIMKQTEVLLQPNPNARIEEFVYEKLDRKAPSRINNPELLGQYMIDAGTEFGPGTAYGNALIKCGETQKRIGTADRELIQTSALNFLTPLRNFIEGDYKTIAKERKLLQNKRLDLDAAKTRLKKAKAAETRNSQLNSARLEGDNIMVNFSYMLNFLHVKWLKIWAEEVTKSEQELRITQSEFDRQAEITRLLLEGISSTHAHHLRCLNDFVEAQMTYYAQCYQYMLDLQKQLGSFPSNYLSNNNQTSVTPVPSVLPNAIGSSAMASTSGLVITSPSNLSDLKECSGSRKARVLYDYDAANSTELSLLADEVITVFSVVGMDSDWLMGERGNQKGKVPITYLELLN

SEQ ID NO: 4 - protein, 394 aa, Homo sapiens
MNIMDFNVKKLAADAGTFLSRAVQFTEEKLGQAEKTELDAHLENLLSKAECTKIWTEKIMKQTEVLLQPNPNARIEEFVYEKLDRKAPSRINNPELLGQYMIDAGTEFGPGTAYGNALIKCGETQKRIGTADRELIQTSALNFLTPLRNFIEGDYKTIAKERKLLQNKRLDLDAAKTRLKKAKAAETRNSQLNSARLEGDNIMVNFSYMLNFLHVKWLKSEQELRITQSEFDRQAEITRLLLEGISSTHAHHLRCLNDFVEAQMTYYAQCYQYMLDLQKQLGSFPSNYLSNNNQTSVTPVPSVLPNAIGSSAMASTSGLVITSPSNLSDLKECSGSRKARVLYDYDAANSTELSLLADEVITVFSVVGMDSDWLMGERGNQKGKVPITYLELLN

SEQ ID NO: 5 - protein, 373 aa, Homo sapiens
MNIMDFNVKKLAADAGTFLSRAVQFTEEKLGQAEKTELDAHLENLLSKAECTKIWTEKIMKQTEVLLQPNPNARIEEFVYEKLDRKAPSRINNPELLGQYMIDAGTEFGPGTAYGNALIKCGETQKRIGTADRELIQTSALNFLTPLRNFIEGDYKTIAKERKLLQNKRLDLDAAKTRLKKAKAAETRNSIWAEEVTKSEQELRITQSEFDRQAEITRLLLEGISSTHAHHLRCLNDFVEAQMTYYAQCYQYMLDLQKQLGSFPSNYLSNNNQTSVTPVPSVLPNAIGSSAMASTSGLVITSPSNLSDLKECSGSRKARVLYDYDAANSTELSLLADEVITVFSVVGMDSDWLMGERGNQKGKVPITYLELLN

SEQ ID NO: 6 - DNA, 50615 bp, Artificial Sequence
  Description of Artificial Sequence Synthetic polynucleotide
tactttcaactgaatttatatgtatctatatatagatatatatagatatatagatacatatatatgtatttttgatatagatttttttaaagatataaaaaattgaaatacttatttttcaacagggcacatttcatttatcagtctctaatgcatttacggagaactaaacgacccagttctagctaatgagatgtaaaccaaagtttactacgtggggtttatgaaaaagctgttttcttgatttaaggagtaagaagtagactcagttggcatgtacttttgcctttacttttcatcctttttctgccttaaaaacatatttaatgcctagaattacaggagccatcttgagacattaggatgaaagctacatgataagaatagaaaaacaaaaacacagaagactgcctacctctgaacttcttagagggtgagaaaataaatcagtatttgggtaagtcactacagctaggtttctgttacttgagctgaacataagcagtccttaatcaatacagactagataacatgaaaactcttctactacaaatacttagaaatgctgggtaaaatgcagcaaaataaattaattaattaataataatgcatacttgagcttacaaaaaaaaagacagggaaatattcaggtggcaaaaataaaaaaagaaactggacaccagggtcttgaatacagaatatgaccctgtctggagaggagaaaggattgaatgaggatggtgcttgaatgtttaaatccatgaggtgataaatggattttaaaaatgtgatatatgtatacaatggaaaactattcagcctctaaaaagaaggaaatcctgtcatttgtgacaacatagatgaaccttaaagacattatgttaagtgaaataagccaggcccagaaagatgaatattgcatgatctcacttaaatatggaatctaaaaaagttgaactcaatagaagcaaagagtggaatggtggttagcaagggctgggagagagtgagggagctggatgacgctggtcaaaggatatacaatttcagttacataggaggaataagttcaagagatctattgtacaacatagtgactatagttaacaacatgttgtattcttcaaaatcactaagaacagactttaagtgttctcaccataaaaataagtatgtgacgtaatacatatgttaagtcactcaatttagccattccaagatgtatacatattacaaatcaggttgtatacaatacagtatatacaattatcatttgtcaattttcattaattttttttttaagatggagtttcgctcttgttgcccaagctggaatgcaatggcgtgatctcagctcactgcaacctcctcggtttaagtgattctcctgcctcagcctcccgagtacctgggattacaagcacccaccaccacacccggctaatttttgtatttttagtagagacagggtttcaccatgttggccaggctggtcttgaactcctgacctcaagttatccacccacctcagcctcccaaagtgctgggattataggcatgagccaccgcgcctggcctttataatttttaaaaatacacggtaacttgtgataagaccttgggcccttggtgcaaacagaaatcagttaactgtgagcacaccctgtcccccaacttcatgacctcacacacacacataagttaaaaacccatgaaattctgtaccctcaatgaaagggtagactagatagaagcttttcttctggcatagtaagaagcttttctgtcaaggcacaagctctgaaagagaaaaaaacaaggaagatctctctcccttgagaatttgaatcatagtttgtactcccacaaatctgggttcaaatttataatatctgaatgatcttagaacttctaagccaagaaagaaattcatgtaaaaattgctcactagccagtgataaccttaggggtctctgacagaagaaacatttttttctgtatcactaaaaatgagccataaaaaaaaaagctaaaaggaatcaatgtacaaagcaaaaggaatcaatccacaaacaaaaaggtcatcaaatacacaaatagaagtattagacccctaaatgcaaaaggatttatctatatgatatcatttatgcagtttaaaaatataacatagtagtaccattcatagatacatataaattcagtgaaagtacaaaaacataacagatattaatcataccaatttaaaaatagtggctaactctggggaaggaatagtgtcaagaaaagcctgaataattgatccagactgaaggagaattgagcaacaagctaactggatgcagcacatgatctttcttggctgggacgtttttgctgtaaagacattggaataattggcaaagtttgagtggggtttctaggcaaagggtacacagaagtttttgtactattcttgcaacagttttcttagtttgtaaattgtttcaagttaaaaaatttgtttttaaaaagaggatgaaaatgaaaaataggaaacagtttcagagaagaaattgccaggatagaccaattggtggtgggaaataagaggaaataatcaaaggggactcagatttctggcttaggtgatttggtgtgtatgattcaatttatcaatgtaggaaacccaagaaaagaagaattttcaggaaaagcaaactataacagacttgccactggaaatattgaatctatggataaatagttggaaatttaggctgggggtgatggtgtcacaaaagagcttagcactgtaaacatcttgatttgattacttttttcacttactaaatcatttgggaattcactatgttgcctgtgtaaaccataaaatatataaaaataaataggaaacggacactgcccttaagggactgatatctggtaagagaagtagaatcagtctgtaggcaaagggtagagactgatggagcaagttctagagggtgacaaattagttctcaaccttggtcacacattaagaacacctagaggaactcctaaaacatcccaatgcccagtccacactctgggactggatccaggcatcagtggttgcttaaactctccaggtgatgctgatgtgcagccaagattaagggagcagtggctctactcagaacacctgagacacctattcctttgaaacatttggcaatgatgcaggaaagagtgtggagagaacaggaggtggaaataaggaaatttggttgaagaggcttctgccaaacagagtgatgatttatattttctctgtaaagaaagcgaatcagctgggtgcggtggctcacacctgtcatcccagcactttgggaggctgtggcaggcggatcacttgaggccaggagttcgagaccagcctggcccacatggtgaaactctgtctctactaaagatataaaaaattagccgggcgtggtgatgcatgcttgtaatcccagctacttggaaggctgaggcaggagaatccttgaacccaggaggcagaggttgcagtgagctgagatcacaccattgcactccagcctgggtgacacagcaaaactctgtctcaaaaaaaaaagtgtcaaaggctaagaataaggatatatgaaaataaggtaagtcttttatggagaatggggaaacaggattacagctacagagggtgcaaaagaaagaagtaacattgttgttttcccaagactgggaagattgaaacacttgaggatgaggattcagggacaccactgtgtccaccattaaagaatttcccctttaatggtggacacggtggtgtgatccttttgtagagacgagcataggttatggcaaatatacctactttaaaaacagtgctctgggaagagagtaagtgcttttccacataaagatctaaaatagacaataaatgagaaatagggttttttattatatattcatttttccatatttctggaaaatgaaacagacatggagtatttcaaagctgggagagatcattatattggagttaccttagaaagagggggctgtttctgccaccacaacaaagaatgaaacaacacaaatatttacgttttcttcttgtcctcataatgcttttactgcactcgtctttcaacctttaacatctactgcatctgtgactgttgtttcagtaaaacacggaagcttgaatgtaagctcatttcattgcctgctgcacaaccacacaaaacagcattttcttattcttgaaatatgatagcaacgtctccttccatgcttgtacctgttttaattcctttcaagcagaggctgtatcgtgaaagatgcttaagaagttacttggaacgacaggatttggaacggatgtcacatcatattctcatttgttattaaaattagtcacgcttaattgttaaggactatcctgtgtcgctcgccatctttgtgtcctccacctggcactttgctttcacacagtaggcacatagcgttgaggtcccggaaggttgtcaagtaaccggccaggagcttgagggcaggacattgtctccgacacttgattcagtgcctggcacacaggaggtattcaattatgctgaatgaatgagcaaccggctcggtgggggacagtaaacccaaccctattccgcgaacgtttgctgagcggcatccaggcctcagggaccaagtgtgtcgcgcggtgccataccgtgaccgggacctgttatttctaatgtccagatattgtaattctatgctgtaactcctatatttcctcttaaaacaaaaagagaaatgtgggtcgtgttcgctctgggttctcgttaagtgcaagcatacagaggcgccgagaccccgcggctcgccctcgccgcttcctccacaggagccgggcggagctcccggcgcgccccggggctgcgccgccgcaatcctcaaggggtcgctgttcccgcgataacctcgagaaaggctccgccccgcggcccgcgcttgtttttcccttgggacccgggtccacacggcggggtcgcccgtccatctccggctcgcccgcggggcccatcgtcgacgttagcggccgttctccgagccgactgacccatccttggcgctgccgccgcgcgcttgttctcctccctcgccccgccttcatcctccccgttcacggaaacgacagctgcggctgcggggctggcgccgcctccctccacctaccacgtctgccctcgccgctctagccctgcgccccagcccggccgcggcacctccgcctcgccgccgctaggtcggccggctccgcccggctgccgcctaggatgaatatcatggacttcaacgtgaagaagctggcggccgacgcaggcaccttcctcagtcgcgccgtgcaggtaccctggtgctggggggaaaaggggtggcggcgcccgggaaggttgagggaggggaccgcagctcgacgcggcgcccccgggcctcgccgaccacccagcgggcctgctgcagccagaggcctgggagatgggcgcggcctggtcccctccccgggcccggcggcggatttctgccccgctcctgcccaaccgccgctccctgggaagcgaggcggggtggggacggggccgggagcttcctcccacccctggggaacactaaggctgcactgtccgcctttctgataccacgacgtcagtgctcctgtggcctccaccccggggctggcggaggcttgcgagtcctttgcggtaaccgagacacaggccattcttgcactaccccccaccctttcctttcgtctctcaccctcccctcggagtccaagaagggagtttccttttcctgtccttcaacccagagagctgccaacacagttccctccggccctctttctccctgaaaggactgtcctgggcttggcagcccgcgggggaaatggagatcgccagttggcgttgagactttgccaagaccacaagaaacctagggttggggggttttcactctttttcaaaatgggaagcaccagctgtagatcaaggagagatgcggggatattgttatggctgtagtcacagtagtatgttttaaatatactttaaggggaaaagttgactacctctcgatattctacaatcaaggaagtgtcagtactaaggaacttgggccgtttcccacagtcatcacccatgttatgccaactcagtaaatctgtgaacagatgtaaatttcacaacatgaattataaacaatctggtgtttacggtcacggtagcaacaacattctcttttgtaaaaagaaaacaaaaataattttctaatatgcataccaaattggtgctgaaatgtaaatctgttctgtggctttactactttccttgcctttgccccaattttgttggcacagttgatcctacattgcactatccctggcaaaagagtagtagagctagagcaaggtaatagctgcctatattatgaaaaacctgtcaagcactttgtaatgcatttgacatattatctccagtcttttccataactttgccagatagatgttattaaccccttttatatatgtgaggaagcagccttactgaggttaagaaacttgcacaactagtaaataaccaggtgctgtctgacctagtactcccagctgctttttcttccgggataagcattccagatttaagatagctagaataatagaatgctgtggcctagagagaccttaaaaatcatctggttcaatctgagcatttccatatcaacaagcaagtaacactttagagatggtaaattccttctagcttttgatttctaagttctaaaatctgcatggtgagtaataagctagctgccttaacccttttcttcccttccttccctaaaatatgatatattgtagacgttgagcctttatagttaaaataatgttttgatttaaattcactagcttctaatagttcaaagacaaggtgcaagaagaatgagtgattgagggtgtgaaatgaattttgatgtgtgagacacttgtacacttttaacattgactgcaatagaggactagagcccttggtatttttgtttgtcctagtgctcattacctataattctagatgcttatgaggatttgacactgtataatgctactcttttaaatacagttaaatatgtttcagacgtataactcacaccccttaaagaggacacatttactagaatttagtctgatttaaattgtggatagactcaatttaaaaaattaaatctagcaaagctctcaaaatgtgtacttcccaaagaattttaattccttgtttcaaattctttaggaaaaagttttaaattttatgctaatctgttagccatcaaataattttcttttcagttttaaatatttaagtgtctactgcatgtctgatgtctgcttgtggctactgtggacactggtgatacagcagtgaataaaatcagcaaagtgtctgccctcctgaagcttacattgtagcggaagataagcttgtaaatacatcaggtggtgataaaaatcatggccagaagaatagaataacgcagagatggtggttgttaatatctttattagtattttctaacattagtatcgtgtatagataggatggtcagggcaagtctttctaataggatgaaattcaagagacctcaaggaagtgacagacctagttttttttggaataaaagtatttcaggcagcaggaagaagtacaaagactgaagtttcaggggcattaaggagaccaatgaaggagctgtggccataatacacgtaaaaggcgattgtaacttagattacagtggtagcagtgaaggttccaaattatccagtattggctgttggattggctgtgaggtatgagagaaagatgagttaaggatgactccaaggttttggcttgagccgtttattaagggaaaaattttaggagaaacagacttctttaatgtgtctgtaattgttagagatacatactgaaatgtttatggataaacttatatcatatctgagatttggtttttacagggtatttttttttttttttttttttgagatggagtttggctcttgttgcccaggctggagtgcaatggcgcgatctcagctcattgcaaccttcgcctccccgattcaagccaatctcctgcctccgtctttcaagtaactggaattacaggcaggctccaccacgcccagctaattttgcatttttagtagagatggggtttcaccatgttggtcaggctggtcttgaactcctgacctcaggatccacctgcttcggcctcccaaagtgctgggattacaggtgtgagccaccacaccagttgagattagcgtttgaatatttcaaaagaaaaaaacagaagtcgctgaaacaacagcaacggaatgttgatagttgtggaaggttggttatgggttcatgaggtttcattatactgttatacttttgtatgtttgacattttccataatacttttttaaagataaaatttgtaagaaaaaaaggatgcttctaaaatagaacatgtgaaatttggaatgctgtgtggtagttttcaacaaaacactgctttctcatctcagtatttatttcatcactgagttcctacattaaattgcattgacatcagtataatgccaaagtaccaagtttcatgctaagaatagcttaagagctatgccaatatctctttataagcagtaagaacagtagagagagaaacattcacgtttataaataataattttcattcaaatcaaattgtaccgtaaggaatatgccattctttggttggccaacgccatacattgttttccattttacttaaaaatttctaagttaaaaattaaacatgtttcttgttttaaaaaaaggtcagctcaaactagtaaagcacatagatgcgtcccatcaccactccacttccctccttttaaaatttgtttataagccttggtactttaaaaagaatgttagactagaatgtataaatgagtaggaatgtttattcttaactagcccatttgggtgcttgtttttaagtacctcaagatggtaaaatacaaaagtatcaggtaattcacccttgcttaattgttattttactagagttttaaatgccatttattcttagaatccatctttttagtccaaacttcttagttgagcattaaatactcccttacaatctggtcctttctctcacttatataaacactgtattccaaattattttttaatttccaaaatatcttctacttattttaggcttttgttcatatggttagactcctcttaagtgtcctgtctgcctattctttcctttccttaagatacaactttattcatctccaaaatggagagagtaaaaaaaattttaaaataaaagtaaaataaactttttttaaagatatagcttcaaatccatctcagctgataaggcttcttagaccacaagagcttgctgtgaaatcgtgaatcattttcaattccaaatgatgaggtggcacttttcttctaatgaactgttatgttagatctcttggaactctaaagttctgttactctgactttctttaataagtttgtatgtgctaaggcatcaactctgtttcttattcccctcttacaacatctaacatggttcatcttttatatcattttagccaggcaaattaccatccatatgtgaagtttaatctttgaagggcaacaataagcatgttaaataacagtatctttattagtattttctaacattgtttgcggaccttgaagttaactaataccagtataaattgagtacctcctaggtgcttggcgttgaaagatacaaagctgaatgagaccctgttcctgccctcagagacttcataataatagtctcaaacttgtcataaatcttatttgtactaaatagccattttgtgaataactttatttgataaatctttcaagtggataaaggaacaagagatgtgtttttaactcagaattcttattgaagcaaaaccttgctcaccatcattaaaataagagcaaagactttctaaaaagaatttttcaaatcaaattacttagggagggagttcattttaattttctaacatttcctctaaactgattattaaagtcatactatgaaattcgtgaacaccagctattttctaaggaatatagttcttaaaatatagttttgagcttggccttgatctcccatttatttcagcaagtgtcatggtgtgtctaaggatgtaaagatgaatatgatgttgccgttcaatagctcctgtcagaccctccctggagtttcagtttggccaaacaactctctcaagcacttctgaaatcctatttcttttacagaattcacaccgtgacaacttcaccatgtttcttaaattctaataaataaaatatgtcacattcgaaatccatacattttcttgttatataaaaactctacatgtttagtgttacttaaaactgttttcacatcctttggtgctatttgcattactttagtaaaatagatagaagatagtttgttaacctgatgttaaaatttacacttatgtactacatgagtaaggtaattagaaaatctataaaactcttttaaattcatcatatttcttgaaagaattttttttttttttttgagacggagtctcgccctgttgcccaggctggagtgcagtggcacgatcttggctcaccgcaatctccgtttcccaggttcaagctatcctccttcctcagcccctctagtagctgggattacagacacgcgccaccgtgcccggctaaattttgtatttttagtagagacggggtttcaccatgttggccaggctagtctcgaactcctgatctcaggtgatccacccgcctcagcctcccaaagtgctgggattacaggcatgagccaccgtgcctggcctttcttgaaagtatttttaaggtaatgacagagctacaaattaatcctaaactcttaaccacacccaactgatgtactcctagttgctttgctactatatttaaaatgatcaaacacctaagtcaattactttaatattctttggagaccagcttactgaaagaacctcaacacagttaatatatttgaagcaaaactccagcacagtgaagtaacagtaataacagctgaaattcctaaatgggccaaagaggaaagtaaagtaatgcagcatgcactaaaccaagcgatacatatttaaatagttatgaatgagtttagaacagtggttctcagtcttggctgcacttaaaatgcttggggaatctttataaatgccaatatatgagctacaccccagtccaattaaattagaatcttaggggttggatcagaccagacatcagtgttttctaaagctccctcaagtaattctaaagaacagccaaaattgggaaccaatggagaaagatgcaccttacagtttactaatacattattcagaatctgtgttacatattttccaagaaatttaaattacttttagagtattaccctttagtaatactggtgattagagtttgcattaaatagatgtctttgatcacacacccattttatttgtagtcttttttttttctaaaagtagagtttggaatccaggctccttatttaataatgtgggttatatgcctatcctgtagtaaaagaaggattacagtgcctggtataatgtagccgactcacagacaattctttccgcaaagcaatatttagagaaaatatataaaaggggattaaacttttattcttatgccactttgccatattacaaatgaaatatgaaaaaattaggtatcaaattatattttagaaaacattgagtttaatgacttactaagaagatgcttatttacagtttgccttttattttaggttgtttacacaaaagttcttaattattaaactatactgccatctattcattaaaagatatgcttaaaatcaactagtttctcattaatacctattatcaagagattacaaaatcagtaatgatttttgacttgaagatgttgaaaactaaaatttttatgggttaatttaaaattttttggtaactaggaaagggatctttttttaaaagaaagaattaataggtcttttgcccttctgaaaaggtgccattattataattttgtttgagtcaaggcagtattctcctagtagatcataaaatccttgagggcaagaagtacatcagatgctgatcatcttgctgtagaagccataaatatttttgattagtaatttaaatattaatttagttgagatgactcctagtgtctgtagtagacgcattatttacagtgctttagagaaaatgtattaaatattgtatttgttatcttaataaagtgtacttctgattaattatggaaaaatccttttgcttccaccaatgataaaatttgggaaatattatgtcttttacaattatatatttaagtaataagatttaaatttataatttcaattttaaaatatgatagcattagcaatgtccttattgtgtgtcttcctcccttttattcattcatgataggatttagtttactaattttggtgaataattattgaatatcttgtagaaagcagaagtgatctgggggagaaagctgtccagagtgctgcgaacagacataggagaaaggaagcacagaatatgattggataatagtaatagaaatagaaaatactaattttggaaatcagcaagtatttactggaagggtagtatagcatgataagactatgactcaaacagatgtgagattgaatcccaacaatgacaccttggacaaagtgtttaatcaccctgagtctcaatttattaatctctacaattattcttactttattttaagattctttcgagtatttgttgtagaatgcacagcttagttaatggaatttttgaacatacactaagccttgacacagaagtatgagaaagatgtaaatggatgtgctgaaatgtgaaacatagaaacattgtcatgtgaccctacagtactgctattttctaaacaggttttctgtaattagactgtctcactcatgtgtaagggttccatgtatcttctgaactaaaatagttaaaaaaaaaaaacctactaattgtgtgtgtttgtagaatctttatcagttaacatattttctttcttcctcagactcatacaaataaatcacttggtgcaaatacccatctttgctctgttatactttcatactcttttcaaaaagaatatatatttattgagatacctgggaggtagtatcatgagagtcacaaagaaaatgttaaataattaagcaaacaggcctttttacctgaagattgaagtgttttattttccaaataattaactttctaattgctgattttcctgaaatactgtcgggagaaaacttgccctgaaccccagtttgtatcagataaattcagtgactattttattttatatttatttattttattattaacttattaattagagatagggtcttgctgtgtcacccagaatggagtgcagtggagtgcagctgggactacaggtgtgcaccaccatacctggctaattttttggatttttttgtagagacgtggtttcgccatgttgcctaggctggtctagaactcctgagctcaagtgatctgcctgcctcagcctcccaaagtgctgacattacaggcgggggcccctgctcctggccataagtactgattttgaattattgatctgttgatttttctattaatctattcttggttattaactataagttaaggatttccacagagaaattaatatttaatatgtgtgagggtgatgctaatgagcttaaagaaattgtgcaactggaagataaatcatcaggactatacactctttccttagatcttaacatagttcatatcattcagtcacaggtcgtttgttacctactcagagatgctgtttctctaactacccgcatctcacactcctcaaactcctgctcccctttctaggcccaagttgcccatccctcccccactgaagccttccctgattctcctgctctccctcttcctctccctgaagaatgaattctttctctgtacttggatttgtatgtcactcagaaacaaaccaaccatgttggtccgctttaccatcttcatatctttatatccttattatcagattatttatttatctattgtctgtctttgccattaaaatgtaagcttcctaagaacagaaaccttatatgttttgttggctgctgaaacccatgtgtttagaagagtgcctagcacatatgtaggtgcttaataagtatttgttgaaggaataaacaaaatgaacaaacaaatggataaacttgcttattaaagggaaatttatgcatgcagtttcaatttttaaggggctaagtgaattttttttagatcactctttataggtgatgttcacctgagtgaaaggtattccctttcttcccttcctattggtcactttatctgggagttattttatctagccattggtggtagactttgaacatgtaaaagtctagtaggaggtctgttgcttagaatcttcagggatctgtaaagactacggggttctctgttaagaatgaggttgcatagtttatccagctggccttgggatcagggtttgcttttgctcagtgcccagtcctccagctacactgcaagtttatgggcttaggcaatagctttgtaactgctatttttggttctacccactaggtcaggggctggctatagaccattctcaaaaagacccaaggctttacaaaagctatttaagctgttatttctagcccctaaacaggagcctcagtggagattgtaaccagaagtctaagaatagaattctagaggatccttcagcttaaggtgttgataataacctgaaagtattaaaccaagaagaaggatgctgaaagaaacaataaaactcagaaaatagagtagcaaacgagatttttgagaaacgaaaggagagaaaggcaaggtgaacactgttttgtcagccagaagtgagtcattactgagcacagaattaatgctagtgatatacctgtgttaagatgggaggaagccacatattctagaggaaaatgatgcatgataaacagcttgagtcatcacctctaatgtttaattaaaagtatccagaaggctgtagatctacacagaatctttttactgctttattattatataggcagtgctaccttttctcaaataccagtatattcaataataaaaattgattcattgagtacgtttaaccacattttatgacctttaaaagatgtcttcatagtatgaataggatatgttccaaaagttagtctataaataaattgctggaacatatcctcagcagcttaaatgacaggttttatggtatgttctgggaaactatttatctaactaggtgtgattaaagtgctaataagacataaaccacaataaacattaacagctctttcaaaacttaataattacaaggcaccaaacaaagtgatttgcatacacaatcatacttaatcccccaaacactataaaatatgtattgtctctcttttataatagagggtactaaagaaaaaatattcaatacattgctcagctcacacagcaaggaacaaagttaagatttgaattcagattcaactgactcaaagctcttactctttaccgttatcctagtctacctaggataggtagttgaaatagttgcatgctttatataccaagaagtatacttaggttttctgtagaattcacaccatacattttagcagaactaagggtatgtgatgactgtaaggtagggtcatccacaaaagaagtatggataaattaccataggagcaaaaaaaagtattcataagcaatggcttacaaagctggagcatggttatttgtatgtttaggattgtttagctttgaatttgctttttacttattgatatggttccctaaaatttatttgcagtatatttaatttttgttttacaatcaataacagttcacagaagaaaagcttggccaggctgagaagacagaattggatgctcacttagagaacctccttagcaaagctgaatgtaccaaaatatggacagaaaaaataatgaaacaaactgaagtgttattgcagccaaatccaagtaagaaactctacctcttgtgtaccttaagtactattagtgggtttttaaatgttaaaaaaaaaagttttaatggccatctgaaaacataggatttttttattgtagcttcaaggtcagtgggtttttttgtgtgtgcttttgtgaagaggaagtgataaacagctatgtacctaaccaattttaaaatgatatttttcagtcttttatagaatccttgtaaaaaatctcaattttaggcagtgactttgaattttaatttatgagcagctgtgtatataaatatgttttcctttttcatctagggaagaattgtccctagataattaagacaaagttatatagttgcttaagtaggtaccattttttatgtttaagtttttggtgtgtttttttgtttgtttgtttgtttgtttgtttgttttttgagacagagtctcgccctgtcgcccatgctggagtgcaatggtgtgatcttggctcactgcaacctccacctcccaggttgaagcaattcttctgcctcagcctcccaagtagctgggattacaggcatgtgccaccacgcccagctaatttttttttgtgtgtgtgtctttagtagagatagggtttcaccatgttgtccagcctagtctcgaactcctgacctcgtgatccacctgcctcggcctcccaaagtactgggatgacagccataagccactgcgccccgcctatgtttaagttttacattttcacataaattatttcatttcatcttcagacaactatgagattttcagataagtcaactcaataatcttgcagttaaggtgcataattgtttagtgaatgaaactcatgagttagcatcttgtcaaatgtagcatagctaattagaagtggaaattggactagaaccgggtgtatgaacatccagtcttttcactgtgagctttgcatatcaaaactgaagaacaaaggtacaatgtatgctttccttcttttttccccaacttgtttgggaccaagttgtgatatgacatatgcttacttttagatactaacttaaacttcaagcactatataaatattagcagttctctatcattttagttaaatagtagtgactttttaaatacaccttttggttgaaaaacttaactgttgagttctgtgctcagtacctgggtaacaggttcatgtgtaccccaaacttcagcatcattcagtatacccaggtagcaaatctgcatgtgtactccttgaactaaaataaaagttggaaataaaataagctttttgcactttgagtgagtcctaatgtctttggacaccatgtaatattttttattcatttaaggccatttaaattctacactctggcacttttattaaaagtaatagacatttttccccttgttctaaacatatttttctgaatgggaaataagctcattcacaactggtaaacagggaaataaaatacaatttcctcctgcccctgttttgtgccaggaagtcgtaataattgtttttgttttttgtttgtgctgtggtttttttgttttgttttgttttgttttgtttgagacagagtcttgctgtgtcacccaggctggagtgcagtggtgtgatcttggttcactgcaacctccacctcctgggttcaagcgattttcctgcctctgcctgctgagagtagctgggattacaggtgtccaccaccatacccggctaatttttgtatttttagtagagacagggtttctccatgttggccaggctggtctcgaactcccgacctcaggtaatctgcccgcctcagcctcccaaagcgctgggattacacgtgtgagccaccatgcccagcctgtgctctggttattaagttacataggttttgagcagccaaacctaacactccttttcttgtaattcctatgttgttttcaatgtatgatcttctggaaatgggtttttcaaaaatacgttatgcagaaatacatattacattgtagcagagttgcctgcatatttaagattttattataacctattgaaattaaacatttattagtattccaaatattttattaactgaacgaagtcagcataagattccattttaaacttttggccagataggtggaaaagcagtcaaaattaagaatcagagtcaaaacacaaagctgtttttttttttttttgagacagagtctcactctttcacctgggctggagtgcagtggcacaatctctgcttactgcaagatctgcctcctgggttcaagtgattgtcctgcctcagcttcccgagtagttgggattacaggcatgcgctaccacacctagctaatttttgtatttttttttttagtagagacggggtttcaccatgtcagccaggctggtctcaaactcctgacatcaagtggtctacctgccttggcctcccaaagtgctgggattacaggcatgtaccaccacaccgggccaaaatgaagttctgaaatttaactagtatcttctaaatggttatgttttaaaaagctctttttggttttaactatttaaaggtatttaatttctgctttaaaacttctgattttgtactagaatctaccacaaaagcaagtatcattgctatactctattttatgtattataactaagccttttaccaactcctgaaatgtttctgttactggttcagctcattttttaaattttcagtgcactgtgtgaaacttccgcatgcgtaggaatctatgtgaggtacttagagggaacctatttgacttagcttcaatttatgcttaatcgttgtttctggaaacttaagccattttttcacttgtcctgaaatacctctaggttggacagcattgtaaatgttaatatctttggtcttaagcattttgctaaccaagtgtttattcagcttttgtttttttcccctttccatttattctaatgttctattattgctttaacctttcgtattttaaacttatttttaatgatgaaatcttttgagtgaatgcatgctcagagatatacttgtatactttaaacataaactaaagccaaggcatcatattgctgatctgtctgtagactattccaagcctggattgttctatgccttaagcgcctgtgtctatatttagactctaggcaagaaaaagagagagcaaatgctcatgctaaagctgtcagatgtaagtctttggttcttccaggtactttatatctgtattttcctagccagaaatcctgattttgaaattatgaatgtacattcagagtaaaaattagagttaaaatgctacaaacagggcaaagacaccttcaataaatattaagtgaattaatcagtccttttctggaacttaaaatcttgtagaatactcaaataataattttattttattttaaaatgcttttgtgccaagatattttatcaaatttctaaaatataattttttagttatataatagtctaacataaaatttaaaaaattatttattaagatatgtgcttgaatgaatgctaactttaggagatgataaatggctgctgatgggggaaaaaaacaatcttctaattagttttgctttttagaaatcattggtaattttaacctttctcctagatgccaggatagaagaatttgtttatgagaaactggatagaaaagctccaagtcgtataaacaacccagaacttttgggacaatatatgattgatgcagggactgagtttggcccaggaacagcttatggtaagtgaaatgcaaaaagttctaataagggatatctttatgtttagatttattagagatgtcattaacatattagtaggccgggtgcagtggctcacacctgtaatcccagcactttgggtggttgaggtgggcggatcatgaggtcaggagatcgagactatcctggctaacatggtgaaaccccgtctctactaaaaataacaaaaaattagctgggtgtggtggcagacacctgtagtcccagctacttgggaggctgaggcaggagaatggcgtgaaccttggacgtggagcttgcagtgagccaagatcacaccactgcactccagcctgggccacagaacgagactctgtctcaaaaaaaaaaaaaaaaaaaaaacatagtagtagaattggttttaacttcaaatttatgttaaaaatttttgatgaagtgagaaatataggcaaatatatacagttaaatgtaatatttaatatattcaagcatatatttagttattcagagaattcacatcttttttagttctaaaaagtttaatgaattcttcacttgttctcccagtgatcttacaatacctatgttataatacttgtgatattgcaattattgtttgcatatgtctcttcatttttaaaaaagctttcttagggcttattttatgttaaaggtacttggcacgtagttcttgaacaaatcagtgcttttgttataattttgcagtaatgtcagtaaccaaaaaatattaacaattgttatctctggatatttggattacatttcatttctgtttttctcttatgtatttcaaattttctatagccgtggtttagttacactcgctttattagttgctgtacagactttaaatgtatagccctgaagctcaaagttacatacacatctttattgtcactctaattttctcataacatgagactgttacagatttcagtagcttcttttcctgttgtttatgtctaataataattctctttatacatctgcaagatactggtctttggatatgaaatggtaggtagattaaattaatttctacacatagttctgatttacagaatggtttataaattctaggattaaatgaattttaagctttagtagttatctttatgtcagaaagaaataacctaaatattgttgaatgccactaatccattgacaggcttttttaggtacacaactttgaaacttctacattgtgttcttcaaaatctaatttttaagtttgaaaatgcttagaacttcaaaataaaattatttctgaattttactgttaatttttagaaaaatgttatacattaaacagatgttttattgcctacttgtgccaagctcagttagatgctgggatcataaaggtatgtgaaaggaacccagcagccccttccttcaccaggctgaagaaggttttagcagagcatgaacattagtaaaggcttctagattttcttcccttttaaatctgcttaatctgtttgcacctctagtaattctagtgaatgtaccctggttattagaattgggctttgtgaacctattaccttattagtaattaatactaaaaattgagtatcacaggcatgggctaagcattatatagtatatatatttacatattaattctcatttatatcctgcttttaagataaattatattaccttcattttaaagacaaggaaacaggtttggagaagttaaataattaatccacagtcacagctagtatagtaattcaaatgaagatctgtctgtatccagtgacggagctcttaactactatctgtattaccttccttatcagtcagatcaaagcttatcctaacatgaagggattaagatccttagagctaaagcttattagaagtaaaactaggagtggaaccctgatcttggggcttcctttctttctactctactttgtactgtacttgccctatttaagtatcagagtatatgtattgcaaaagcatggattttttaacttaagatatcactatgaactattacttatagatacttcagatgataactgcaaggatgtagaaacaggacaaattgtgacttgatctcttggaagcattatacttttataagtataaccacagtcacccctcgatatccatgggggattggatccaggcccgccaccctgaggataccaaaatccatggatactcaggtgtcatataaaatggcatagtgtttgcatataacctatgcacatcctccctcgtactttaaatcatctgaatgtaaatgctgtgtaaatagttgttatgctgtattttttaggaaataatgacaagggagaaaagtctgtacatgttcagtacagaggcaaccatcttttttttttttttttttttttaatagtgacaggccctgctgtgttgcccaggctggccttaaactcctgggctcaaacgatcctcctccctcagctttggagtagctgggactgcagacatatgccaccactgtgcctggctcctcagccatccttttttttttttttctgaatgtttttgatcgtggttggttgaatccatggatgcagaacccatagatacagagggccgactgtgaccgtaaatattttggggtatatttgccctctttagtcccattggtagaattggtacaattatctatgctacaataggaagttcatctttgtctccttcatttagacacatactcttaaatttacttttaaaataaaactaccttaattacagttaattctatttggttttactttagcaagcaagttacagctaacttttaaacagaccaaattgctgatttatctttacatgattttattttctggtattaccagacaatgatttttaaaaacatttttcctttgcaggtaatgcccttattaaatgtggagaaacccaaaaaagaattggaacagcagacagagaactgattcaaacgtcagccttaaattttcttactcctttaagaaactttatagaaggagattacaaaacaattgctgtgagttgaaaaatgtcccctttatttagtaaaatcatttagatatatatactttttaatgaataatttggcctcttaatgactgtgtagatgtagtggattacaaaattaaataatctaggtcagtgcatgttcaccagaacacaaaattaaaccaaaataaaagactactgaacatttactccatttgtgtgtcctacagtgttattgagatgcctttatcccttttcattcattaccaacgctaaaatctatagagaggttcctggaatgattatgaaaatatggtctaaaatatcaccatagtatatataccgtgtagctcttcattttttaggattttaacttattttcttagtgatagagaatgaaaattttctaagagttttacaaagctgctcttataaatacttcaaactttaatttgcagatttcctattacaatatattatagaaactttgaatatgttcacaatggatttgtctctgtataaacaaatatttggcctgagacagaagaactatcaagttaaagtatgatttaaaagatattagcagccgggcgtgatggctaacgcctgtaatcctagcactttgggaggtcaaggtgggtggatcacttgaggtcaggagtttgaaaccagcctggccaacatggtgaaaccccgtctctactaaaaatacaaaaaaattagctgggcatggtggcaggtgcctgtaattccagctacttgggaagctgaggtaggagaattgcttgaaccctggaggcggaggttgcagtgagccaagatcgtgccactgcactccagcctgggggacagagtgagactccatctcaaaaaaagtgaaataaaataaaataaaataaaaattagcttatcagtgcaatgaaatttgacatatggaatctaagattaaagcaaactgaattataataaacttacatactatacagaagcttgaaattccctgaagaattctaagatactatactaatgtttattcattattctgtaagtttgaaattccattttggagtggttgggttgcatttggtgctatgacacattaagtgcagcccacccaatccttgaggtaggacaccaggcgtcaaggaacccttgccccaggcgtatacttccttctttctaggcactttctggataatctgtaacaatttgaaagtcagtctggccactaatgacgttttgggctgcacaattccttgtaaagatagactgtcttgtggaatgtttggcaacatttctggcttctggctagcagactccaataacatccctccaccaattgtgacaaccaaaaaggtattgccaaatgttcccgaggcagggacaggatcataaaatattaaaactgatattcctttgtttccatcattgcctgccatctttcccagacacttacaatgtttcaattatttattttaaaatttaaaacagtaacaagggggtgggggggggcagatacatgaatggtaactttatagccaattttgtagtcatagatctttatcaagtgatacatatgtttactagggaataatactttaaatgtgtatgctcttaacagaattttagcatctttagcccttattttttcttttctatatttatagaaagaaaggaaactattgcaaaataagagactggatttggatgctgcaaaaacgagactaaaaaaggcaaaagctgcagaaactagaaattcagtaagtaaatagaaaaatatttttgattaataactttaagatttgtaaaatattttgctaataaaccatgaaagatacagactattaactttagagtcttgtttttgtgctttctaaacaaatttatcagaacttaatacatccaaatcactgcaaagtagtcatatcaaaaaggtgatattcatggccaggcacagtggcgcatgcctgctatcccagcactttaggaggctgaggcaggcagatggcttgagcccaggagtttgagaccagcctgggcaacatggcacagccccgtctctctacagaaagtacaaaaatgaacgggcatggtggcacccacctgtagtcccagctactcaggaggctgagatgggagaatctcttgagtgtgggaggtggaggttgcagtgagccaagtttgcgccactgcactccagcctgggcaacagagccagaccctgtctttaaaaaaaaaaaaaaaaaaatatatatatatatatatataaaatatataatatatatgtgtgtgtatatatataaaatatataatacatatgtgtatatatataaaatatatatatgtgtgtatatacacacacacacgtgatatttagctatatagctgctgctaaaaaacaaaaattatggtagctcctaatgtgtcattttttttaaatatcgtcagtggtcgcaaatatttttggagatagatttattataattggaaatagcagaaaagatagtaattaagatgagactcaagtttgagaatacttgtgtgtgacttaaaaattagatcagttccctttccacacactgctcaaactaactaaaagaatgttcaccaagaatagttccaaaaccattcaaaccaacagtggcagtatggactttcaggcaacgatgttgaatgcgaggaaactcatttggataaataaattctagtatatattccagaaaacctcagaatattgagtaggtatatattaaatataattaaatttttctttaaagatttatttataagctttttagttaatccaagttatactgtctatattattccagaatttgaaaatatcagatttgaattggaataactttaaaatagtataaaataaaccaagtatgaatttgttagaagaagctaacaagttaatacttaatttgagctatggtatgatagtacccctgccttctgtttccaagtcactaccactgccaacatcaagaatcattgcgttaatgagcatattaccacagtgctggtactgtcttatagttctcttgtttgggagataaaaaggttgaaaattggtttagagtgaaatttaaaactgattaacttggtattggatagtaatgtcttacgagactaccctaataccttgtatgagcagaataagttatttttgtctatggtttttttcttttttggagatactgtattagttactgctagagaagagctatctttgataagacattaattccaaatttactggcattttcacttcacagtttatcattacatcattatcacaaacaacaaaacttccctaaacagactatctgcagcatcagaactttgaaaatgaacactatagatagcaggagaccttgaattcttttattagtttctagttgctaataattaatttgaaaagtaaattataaactagtttttaaagatagaaatttcatactcaggaaaaaaggatcatttggaaatagatattttaactgggacagaatgtctttattcaagatgcctatatgtgcccagtgataattgtggaataaaaatactgttaccaagtttcagtacaaaactttgtttaaaaagcactattacaggaatgaccgtctgcttttctacctggaaaagttccagaaagcacacacatatagatttaacagtgtacaaagtcctttaatatttctaaattattttaccactgctattaaaatatatagaacctaaatataatagtagaccaattttgagagatgtttcctttttgagatatctagaaaagatcttggcaatttttctctgctgcgtatttttgaggataaaggttttttttctatgggaatttatgtttaaagtaaaagatgatacaaatgattttattcagttcagcaattttatttatacacagctattctagaacttacgtattacatacagcaaaatttaactatagccaaatactatttttttagtctagacaggaagttcgatttcctgattttaattgtatttttcccctagataaccaacacatttttcttcaaactttatagcagttaagtctgaaatactttgggaagaaaaagttctcttagtttattattacataaagcctggaggtattcctgtatttggtgatcctttttttcccccctgtatttctttgcatttacttgaagaatcactaggagtttgctttttttttcctttctttgatacatttcctggttcctagttggcaactgaagtggttaatctgaccttagaattactttgacttcacgtctttataaatttccatggattgttttgcaactagttactctgctagtaaggtttgggatctatccatattgacactttttaagtaatttacttactagtttcttgtgactctttttaagttctgtgtttactaaatgctaattttgtgcaaggtactgtactgaggagttcatagataaatgatgtaaaatctctgccgccctcagttcacaatctggtacaacagatcatactttttgatgatttagccctatcagtaaagaatttctgcttgtatctaatattgctgttttatcagttagcatattgaagaatggtaaagcaacatcaaaggcatttctcaaaaatataccaattaagcagaaagaaagcccactttaatgtttcacaatgaacaaaaatacagttttgaaaaatagattgtgtatatgataaaatataccagagaagcaagaacaaaaataattacaggttgaatatcccaaatccaaaaacctgaaatctgaaatgctttaaaatatgaagttctttgagtgcctttatgacgctcaaaagaaatgcttattggagcttttcactttagggttccttagccagtaagtataatgcaaatattccaaaaccagaaatctgaaacacttctggtcccaggcattttgcctaagggatacaaaactgtaatgcatttcaattataaggcacctacatttacgaatccaattttagtctaaatctaaacttaaaaattattttacagttttagtaatgttaaagataaaaatcagctgatttcaaagctctgcagtatttagtggtttagacctctgttaattttgggacatccaaatttgatggaaaatatttctttaacatcatatagcaaacttaagcatttccacagcagtgtaaatctttaatctgtaaactcaataaatttagagcaccatcagtatagcctttaagaacacatgtttgaatttaaggtttactagaaatgtaaggattgacatgcctttaagacttcagagtatgccaagacctggctgaggctgattgaaagagaatgtggttgaaggcatcataaattcataaaatagaaatgatctttgaataggaaaatttggggttaggtaggactttcttagtgaaaatttaataaaaattgtaggtaactggcagttttaaaaatgatacattttccctaaatgattctcatttaaagtcaagcatcaacatattttgttttgaaaatttgaattttggggaaaaaaatgaataggcaccactatatgcagtttcacagaacaataaatgtagtgggggttttttaatagaaaaaaatatttgattggctcttttacttgtttaatgtgttatttattgtgaagaagtaatttgtttcactgtgttcattggtataatgtgttctttgtctcttactatgcacttaagcaactaaactcagctcgccttgaaggagataacattatggtaaatttctcttacatgctcaacttcctgcatgtaaaatggctgaaggtttgttggcctactgtgttttctacatcttgaataaagtgccttctcttgctattagccgcttgtaaaattttgaaataattggaaccatgtattcatttatctttaacaacaaaaatataaggaaaaaatactagctgaagtaatactggaactttaaaagaaattgtatgttctgattgccaaatatagtgtttatgccaaatacagattttagaatatgaatttatacataaatatatagtgcctttttgcactgactaaaaggtgctaatttaagctggcatttatattttcacttttggcaaagaaaattaacaaaatatacttaaagaaaggtgtaaggtaaacacattgcctttgctgctcagtagcttcaagatttaccttattttcacccaaactcaacatagaattaattttttctgccatttcaggccaagacagaccccattaggttaggtactcctgtaatggccaggcttattttccacccagtgctaactcacttaagaaagttttggtggaatggtgatagccttgggaaagaatagagaatgcagattgttttatggtatccaatgtatatgttgagcttagtttgagaaaatttttgagctattagaagttttttattttagtaaaatagaaagttgtaaaatctgaacatagccaacaatttgtgcacattaaccaataaaaagtatatagtgtgttttattttatttctagatatattacttcattagaagtgtcaagggatctgcagtacagctaatacattggcaattaattggctgctttcattcactagcattaaaccaaaagcaggaaagtaataggaaagattattgtacagaatatataaaatgcatttttgtttttgtttttttttaaatggtaattactgaagccctggctgaattagggcttaccttagatagcaagagaatgcattaatgtgtttccattgtcctatctataggtaaatgaaaagcgtgaggatatttcagtcattgctaatttaaaatataaagcatagcttttctttttgtctgaggtaaaaacataattctaaacaattaacaggactttgtttcattaatcatttgtgtttactattaatgtaatcttatagtaatatatatttacagctggtcattttatatttattatatctccagcttattgtttctctccaaaaagactgtttcagcagccattaatccttgctttgcttcatttcctaaaatgttagaatttatacctaaatgagaaatttgaaatcatttagtccaaatctctaattttccagatgagaatactggagctaagaaaggttaggtgttctcaatttgggaaaactgtagatttaacataagattttaaaattgatagcaatgatacatataaaagtcatactttcacagtatgttaagctgtaccgatttagttactactgtgtttaatctataaatatgattctgaaaaagagtatgccatttaaacaaatatttatatttttctatactaagttgtagaaaataaattccaactttcatgctttatatttttctctaatttttaatgctggctagatttgggcagaggaggtgacaaaagtaagtaaattattaaatacagaactatgaaacaagaatttagttgacgttgattcagtaaatatgtattgggcaacaacccatgggtccagtcacagtctgtgttagtataccttataaaatctgaaaatgagggatatgttcaggtctctttagctaatagaagctccaccaagcagttgaacaatgctgcagagatgtggtttgcacacacactgataaatcttggatacctgttatactaagtggtaatcttacagttccttactatagaaggcaggcgaaatgcttgttaatgatttttttaataacaatttttaaaatgtaaaatatagttaacaccatgtcctgtatcttcagagaatgggatatataatatttcgcacaaatgaattttttttttttgagatggagttttgctcttgtcgcccaggctggagtgcagtggcacagtcacagctcactgcaacctccaccttctgggttcaaccgattctcctgcctcagcctcccaagtagctgggattacaggtgcccgccaccacgcctggctaattttttgtatgtttagtagcggcagttttttcaccatgttggccaggctggtctcgaactcctgaactcaggtgatcaacccacctttgcctcccaaagtgctggcattacaggcgtgagccaccgtacccggccacagatgaattttttaggtaactgaagcttgttctctccagcaccaaagctaatttttcccagtcacattagttgaaaatatctgtaaaaatgtattttactgctgcattaaatataggatagcaagtatttgattttactacagttaaggtcattattatgaatatttatcatactgttaacagttggatttgttgtttttcgactatgttgtctcttccctcagtatcagactgttgcttctagaagctgtctgtatgactgctgctagcagttctgtctgtgttaccatttctaatcttctgtgggctgggaaagcaagtaattaaacaagaatttaagggataaaattataaactatagtacatgcagacttgtttcaaatgcttttttaagtcttcagaagcatttttcatttacttcttatgtttaggcctaccaaactctgacatccacatgaggataacactggggactgaggacaggtgatgattttggcccctgacaatagctttataaactttaacttttattttctggctttgtaggattgattttacagtctgttttatgcataggttatagagctgttgattctgtgacttggatctccagtatgaaattcttaccttatgctcttatcccccctttgtgagtctgttcatcagtttagttacggttccacactggcttctgatttctctctgaacttctagcatgaaaaggggcagagcaacacagaagagaatacaggttgaacaaaaaaactagaatgtcttcctctttggcataaagatatgccagaaaatttctgacttgattttgcagtctcagcaagtttaggaaataattgatacaatacatgggaagaaactcatgaaacatattatatacatatatataaaaatctaaagaaacctatcaaaatggatttcaggggtttttttcttctacatatctcacttctgacatttttataattaaaaatgtataaatgaccaggagcagtggctcacacctgtaatcccagcactttggaaggccgaggtgggcagatcacctgaggtcaggagttcaaaaccagcctgaccagcatggtgaaaccctgtctctactaaaaatacaaaattagccaggcatggtgtcccagctacttgggaggctgaggcaggagaatcccttgaatttgggaggtggagtttgcagtcagccaggatcagccgttgcactccaagctaggcaacaagagcaaaactccatctcaaaaaataaataaatgaaaatgtataaatgttaagactttacattcctggagtattttaagattttaagccatatgaatagtagtcaacactgatcaagatagtcacagcaaatgcctcagaagcattaccaaaatttagggcttacagatacccaatcttgatatcgtgggccaacagattcttgcccatcttccagttacattttagaaaaaactcttctggcttacctaacctatgttatgctatcatgtagtctagtagcagttacttttagcagtagaaggtcttctccctagagattttttagacgacagaatatcagccctttcttatctatctttctaatgtggatcattttcttccgaagtaaattgattatgctaagcctaatttaaattcctttagcctctcagatcagttattatgatcaattatatatgccagaagtatagatgatttgggacatttgactgtttgaagccacccaatatgcactaaaacttgttcaagaaatgagatgcatttttccctatcatctaccctctctaaagtctagattgtacagaaggacctcctaagctttgtaacatggtgcctttcttcatgattaaattttactcaaacctagaagcctgatccctcatccttaatgagatcatttgatattttttattcagtgtacttagcattaaaatattatttctacttcagtctcaatcttcccggtcaccttaatctgctacctagtttacttgcttattaccttacctagaaataatgtcagttagtaggatttttctgccatttctactaaaggccattaaattaaaccgtactttgacaatcaaagacctatatggaactaacctagtttatttctgaagtgcatttatttttggtttgctgtatttacttctgttactcaaagccttttgtattcaaccaagcagaacagttaatagtgcatatgtaatattgatgtgattggttttccttagtgctctccttaggaagaacattgcttctttttttcttctgggacttttttctacttcataccagcttccagatgaagcttactttgtccctgctatcaaaacaccatgggttctggttttagtcaagtaaatttaatttcaacttctgacttactaattcaagcttatttccgtaagtccaagctagaggtcaattattagtcccctctgctaggtttgttgccagtgggctgttacagcaactgattatctactttgctgctacttccttgtagactggtttactagggtaactattgccaccaaaggatgaggagagggagatgaggcctgaaagaaaaaagttcataccttgttgggttttttttgtttttcgttttttttacttttattacatcttggaaatgtcatgaatcatttgcataggtggaaaatgctatttgtgctggagaggtagtcaaacctatcattataatgatgatgtaaccaatgataattcccaatgggagaaaagctaactattctgagtaagctgtgaggttttatttctctttcccacttattaacaaccaactctgactaaaggtttgagaagatgttttttgccactatttgctatttttagatcttgttgatttttcagtgaactgttttgtcgcagcagtaagcctcctttatcagatggttgaaaccagtagtggcatactgttatttacatctcttcccagctccacagtgtgtgacttcacgttagtagctggaaatcagctgtggtgggagtatttatatcatgaaaatctggatatgagagcttttttttttatttcttggggaagaatttaacagcacactactgattgaagttcgatcataacttaaaaagctttttaaaaccctagtgtatcttaatagatagtattctgcttaagacattttttatgcatagtttgaaaagaaaatttgaaagaaagacatttaaaatgtaggacaaagaatcctttgaatgaacctaaaaactggtgctagcaacaagtgtttggcaactaaacgcaaggtgaaaatcctatggcttcactctaaagtcttttgcatttgagactgaagactctttaacttcaaagtaaaaactgtagtaataattgagaatttttgcagcaatattactttatacttgtaaaactggtcatcttaaaattttgtaaaatagtaaaatttagcaaaattgatactagaaaatttatgaaaatatcttggctttctcttagtagtttttgtaggctcttaagagtgtatcattaagctttagcgtaacagattcttttcaatcaagtactggaatcagttgtccaggtggagtaatacagaaaagctaaggtctagaaaccaggggaccattttagatacaaagcagaattgcggaattgccagattataacttgaggtttgttcgggggattttttttaaccatctaaaataaatgtatataagatggactgagtcttctaaagagattctaaaactatattcttacttaagtctgaacaggaattaagaataactcaaagtgaatttgatcgtcaagcagagattaccagacttctgctagagggaatcagcagtacacatgtgagtattcattcattggaaattcatttggaacaagactttggtagtcctaaggcaattttgggaggaaaaactgaacttttcagtcattcatcaaggaaatttaaattgttctactttccaatatatccgaataaatattttaaattgttgtttacatcttatcaccgagttctcccaccctcattaaatatacatgttgttgtttattggctaagcagagtttgctagtgtactattcacgaaggccaatcttaataagccttgtagtaaaatgttagatactggtgagtctacctcagtacaatattgtggtctgtctcccccttgcatttgccatttaagttgactatacagctaagaactaactataattttccttcacaggcccatcaccttcgctgtctgaatgactttgtagaagcccagatgacttactatgcacagtgttaccagtatatgttggacctccagaaacaactgggaaggtgataatttacttttcacatgtaaagaggagaaattcagatttttgcttatgaactaatactaaggatgaagattagagcaattaaaggttcttactagctttcttagaaatagtttcttttttttaaggaaagtaattatcagttttctgaggtaacagaatattttgagcttttctggcacagaggcatgttaacagggatgagaaaaagtgaataccttttcagtgctgtaaggttaaaagtgtttcaagttcacttttctcctgctactgaatactttttgctattggttgaggaagatataattaaattgacagttacccttaaagataaactaatttatacttattcattactcatctgtttactatattgaaaaaaagttttagaatatttcgtaagacctcatatctaccgatgtgattattttgagtatgttaatatacttcaaagcaaaatgacagtatgtaaaacacaaagctcaatcactcattcatcacgtatttattgactatctgccatgtgccaagcactgttacatattgtggggtaatatcagcaaacaaaacaggcaaagatgcttgcctttatggagctcacattataaggtgacactgaaaataaaataatgtttagggcaaacatggtctctgctctcacaacacttagcctaggtagaaatacaaagcaagccgttggtagacaggccatgtggtaggcagaaaagcagaaggtgcctggtattgtgaaggcatctaactgtgactgaggtacatctagcagagtttcccaggtgaagttatcttggtaggaataatctagatgaagaaagggacagggatgaatattctaagcaaggaagaatatgtttataaaaaagccagaagaaagagagtagggaaagttcggggaactgatagtagtccattcaggctggagcatagtgttcaagaatggaatggtagatgaatctagaggagttaagcagggtcataccatgaatggcttttaaagtaagttgttgtaaggagtttggaatttatatattgaggaaccattaaagcaggggagcgatatgtagcatttgaaggatgaatatcaaaaaatcgtttcaaggaataaggtaaataaagtcactttggtgtttgtttctgcacattaacacttcaaaacttggcctagaataaaattgagcacgtccatgaaaccaattttgaacctcaaaaatgaatggtagcctaatcattagttattttaatagaatatacaggagcatttacataactatatatttgaggttttattagtggtgttaatatctcaaggcagaacagcactttatcttcacatcattataactattttaagcccaagaagccttagtacttttttccactttatcctgaaactctggaagtgcggcataggggttgcttatactcgcattctacttaaagccacactatgcttaggacttggccatttaaacaaaatccagttgttgcgttattactgctctttgagataagctccagcaatacaaagtaaaaccagtgctactattctagaaaagaaagtctagcttgtctctccaccctcaattatattattgctaatgaccaatgacattggaggaagttttatctctttaatttgttgcaggttatgctttttaagataatacatagaaatattcaaatatagaacttgacaaatcatccagcttcacactaacacattcaaaaactatttatgaagaagagaaccagattgtattgttaatagttgcaataacagtaccaacaggtagtagaagtaactgaataacagccatcactttggttgagcttaagttgtgcataggtgctcttccagacacctcatgtgtattattaactttataatcctcaccaccaaatgaggtaggtactattattatccatattttaaagatgagaaagcagaagtactagtcacaagattagcaatttttttaataattggaaggcttttgcatttcaagctccagtggcttaattgacatttttatagaatgagtaattgctatgattttttaacgtatcaataaaagttcttagaaggtaaaaatggcctttcagttcattttttaaatctttaactctgcaaaattgacataggtacacttcagtggctaatcaaaggctaaaatcatactagtagatttggaagtaaaatatggttagagaactaaaaaagccatgtgtatatacatattcatataaatagagctttttcttatcttcagaaatgtatgtatttaatccctactgaagaaccgagttcactgaggaaaggatactaggacaggtggtcttaattttagagaggaaaagctatatttaaaactatttcaccctttatccatcagagaaacatttgcttttcatttttaaaatgtactgttccagatgtgggggatatagtatataaataaaataatcaaaattcctgccttcctggaatctacattctatatgggggcagaagggagcagaaaagcaatatgtatataaattatatagtatattagaaggtgaaaatggtgtaccggaaaatatagcaggaaactactttgatgggagtagagaaagacgtgagaaagcacacatgcaggcatctgagagaagatgctccaggtagagggtgcagcaagtacaaaaaccctgagggcatgacatagctggtatggtcaaggaacagcaaggagggcttggctagagacgagtagaggaaacggggagccaaaacatatagggcttcgactttggtctttactcagagatgaggcgcctttggaaattttgagctgaagagtgacataattttaggctttaacagaatcattctggctgccacgttgaggataggtgttgggtggaagctgactgatctgtttcaaacttattgcaataatgtagtcagtaattcagtcagaagatggcagtggcttggatcagggtaattgtggtaggtaggatggaagtggtcaggtcctgtgggcctgtgggtgttgtttgttttttgtttgtttgttttgttttgttttgttttgtttttttgagacagagtcttgctctgttgcccaggctggagtgcagtggcgcaatctcggctcactgcaagctcctccatctgggtttatgccattctcctgcctcagccacccgagtagcggggactacaggcacccaccaccacacccagctaattttttgtatttttagtagagacagggtttcaccgtgttagccaggatggtctcgatctcctgaccttgtgatctgcccgcctcggcctcccaaagtgctgggattacaggcgtgactgtgggtgtattttagacttaaagccagaaggatttgcttgacagactgtatgtatattggacacaaagaggtcaagaattattgcaaggttttgttttgttttgttttagagacagagtttcactcttccagtccaaggtggagtacaatggcgcaacttcggctcactgcaacctctgcctcccgggttcaagagattctcctgcctcagcctcccaagtagctaggattacaggtgtgcgccaccatgcccagctaattttttgcatttttagtagaaaaggggtttcaccatgttagccaggctggtcttgaactcctggcctcaggctatctgcccgcctcagcctctcaaagtgctgggattataggcgtgagccaccacgtccatctgcaagtttttgacctaagtaactgaaagaatggggagttgtagatagttaaaatggggaatgccagaggaggcaaagatttcagggatggaggtctgggaagtatgtcagttatattagaagcccagttttaaatatgttaagtttgagatgtctcttaaacatttaagtggaggtatccaattttaggatatgcaggtctggacttggattgggggcagggtgaattagataattagagagttgtcagcatatcaaagatatttaaatctgtgagactagataaaccctcaaaggagtaagtatagagaaaaggagaggatcaaggactgatccctgaagtactctagcatagagagttgaggaacgaggcaaggaagatgataacgagtgaggcagtgaggtaaaatggaaactaagagtatggcctggaagtcaagtgaacatgtgtgaaagtgggagagaatggtcactgcctaggtcaagtgagaggactacgaattgaccattggatttaggaacattaaagtcattgctaaccttgacgagtagttttggtgaaacagtaggcgcaaagtaatgtttgtaatataagagactcggaggagaggaattgtgaccgtaagaatacgtaaatctttcaaggggttttggtgtaaaggggaacagaggaatgaggcagtagtcggaggagaaagtggggtctagcaattttttgcttgttttttaagataggagaaataagggcatgtttgtaagctgatgggaatgatacactaggtggagaaaatatgataatgtgggagggaaagggaagaattgctagagtgataccctagagaggcaaaaaaggatgagattttgtgcacatatggaggaattagcatcaggagcatagacagctcattaaagagtttcctggccaggcgcagtggctcatgcctgtaatcccagcactttgggagatcaaggtgggcagatcacttgagctcaggagtttgagattagcctggtcaatatggtgaaacgctgtctctacaaaaaaatacaaaaattaactgggtgtggaggtgcatgtctgtagtcccagctactcaggaagctgaggcgagagaattgcttgcttaagcccaggaagcagaggttgcagtgagccaagattgaaccactgcactacagcctgggcaaaaggaatgaactcctgtatcaaaaaaaaaaaaaggagttcctgattgttcttttttcctctcagttaaaaggaagcaagctcatcaactgagtgtaatggttatggggagaactgctgaaattttgaggatgggcaagagggtatgaaatagtcatcaggggaatgaaagtgaaagtgaatggagtagagaaatgtagtatcattgccaggtggcatgtggacccacttgaggttcctagtcataaattgaatgtaaaacaaagcttttctgtgtttctcctgttaaagtagatgcatggatactgaataggtaaataggtttttaccaaatcagtacaatgaagtgagtggcataagaaatgagagcatatctaagagttattataataattaactatggacatgaagaaggtgttggtaggatcatagaccctggtaggcttgttgatgtgagagcacttgaaagaaaaaggcgatggtcaaagaatgggatagtagaaactattattattggaagggttgtggttactggaattggtaagatctagatgtgactgtgggaataggtggctgaactaggatcaggtatgaagtcactagaagagaagagttcgaggatctaagaggctaaagggttggaagaattgtccatttggatattaaagtcaccaagaattaagtaagagataatgtcagtgagccaggtgataaaatcttctagaaatgaaagtaagggaatgaggcatctgtaggtgattgcagtaagagatactgggtatctctttttttaaagagttgggaatgatacaatagatggagaaaatgtaatatgggaaggaaagataatatgataaaatgtaatatgataaaatatgataatgtgggaggaaaagatgatgtgctatagttgaataacatgaagttgaaggattggctagaattgtttagggaaaagagtctaaaaacagtgatgaagaacataagactcctccattgacctctaggttcagtgataaaagaggagtatgggagataaacgctagcactggaggatgaagggaaggtttagagaagaggttaaggatacagggaattttgctgatgactgaaatgatttccagatggtatagtgctcgtgaagagaaaagaggacttttctagtttaatgaatttattaaaactttgaatttttgtagatcattttttagcttagaaatggttgaattagaatgatgtcagaccagctattttgtactgttttatatttggtattattggggaaactagccttaaaataaactgaaaattctaattatcttcaaattatttatgagcacattaatcacaaaaatcactaatttctaatagaaaatttttctaaaaaatagcacaaatgaacttgtaaaatttgtcctattctaccattgagtcttcaatttcattgagataattagcagtcatattatttgaatgtctaccttctcactatgtgtatgtccttaagggaatagtatcatttcatcttacgtcttctgcttctcatagtcatgctgaacatgtagatacaagtaaataatacttacatgtaaatgacaataaatattttatttgaataggaaaatgtgattggaccagtttatgaaaaaaaggtttagttattcaaatatttctcaaagaattaaattaagaaagcctcatacttgtagtatctaatatttaagtagtttttcatcaatatttttatttagtaatttataaagaaaagattaagaaaacattctaatggaagttttttggggaaaaaagtatagccattaattttaagttaatattataaggacagttttgaaacagactttcaaggatttatatttgaataccacctgaattgtctctagtataattggcccaagagggaaaaggaagatagatatactttgtcattaacgtattattaatatctataaatcttttccaatgtgaaggttggtagaaagtaaacagcgccaccgagtggtggtattaaggagctttagtcacttggaaataaataattcgctgctttcttttcaacagttttccatccaattatcttagtaacaacaatcagacttctgtgacacctgtaccatcagttttaccaaatgcgattggttcttctgccatggcttcaacaagtggcctagtaatcacctctccttccaacctcagtgaccttaaggagtgtagtggcagcagaaaggccagggttctctatgattatgatgcagcaaacagtactgaattatcacttctggcagatgaggtgagtattgtgggtatgagaaggaaaatatatcacgaattgacataccttctaatattataacaaaatgcaaattcctcattagttatttggaaatggtttcatagattacagatagttatattaacataaaattttaattaatattctttaatagaatatcatagaaatatttaaaacttttaactgccaaataacttgaagatgtataaaactccctccaataagatcctttatctaggccagacatggtggcttacacctgtaatctcagcacttttgggaggccaaggcagtgggatcacttgaggccaggagttcaagaccagcctgggcaacatagcgacacctcatctctacaaaaaatatgaaaaattagctgggcatggtggtgtgcgcctgtagtcccagctactcaggaggctaaggtgggaggatcgtttgaacacgaaaggtcaaggctgcagtgagccgtgatcatgctgctgcactacagcctgggcaacagagtgagaccctgcctcaaaaaactaataataataaataaatcctttgtttttcagcacaattgataataacgtttaaagtccttttggtgaataatttaaacaaattaaatacaaatctgattggttttctacttgtttatttttttaatgtagttaataactggaagtattttattttgcaggtgatcactgtgttcagtgttgttggaatggattcagactggctaatgggggaaaggggaaaccagaagggcaaggtgccaattacctacttagaactgctcaattaagtaggtggactatggaaaggttgcccatcatgactttgtatttatatacaattaactctaaataaagcaggttaagtatcttccatgttaatgtgttaagagactgaaaataccagccatcagaaactggcctttctgccaataaagttgcatggtaaatatttcattacagaatttatgttagagctttcatgccaagaatgttttcttacaaaattctctttttattgaggtttcactaataagcagcttctacttttgagcctcaacttaaagcagaactgttttctactggatttttcattaacagcaagctttttcttttatgtaaaataaatctattgtgaattgatatcagcgactcatttattaggtataaataatagccaaagaataaaattaaaatttattttaaactttcggtcttaaaaagagctgtaggatataaacctcttttgtgaaaagtagaattttctgaatgctttcatagacaaaaacgcatttaaactatgtttacctggttttccaacagaaatcaaatgattttaatattatgttttgatgggttattttcagagttgtttggttttttaaacactactgaatggtttcttttaaatttaagaacaactagttcttgaaaagcaataccgtatttctctgataaatttgtagttaaggttctagaaaatctaggacaaatttacttcattaaacataaagtttttaagattattcagttggcacaatttaatgcataattgggattggatccatttactgtactgtggtacagtaaagagaatgtgttcttattgaacagcagatttgtatacaaatacggaataatgcaactacattataaattgtatacgaacaaaataaattgggtcactctgagcttactattaaaacattgaaaatttattaatgaacacaggctatcttgatgaggatctctgcttatgaaacacatactaatgaaattattagtgacacattggaagctagaaaattgctgttggggccattgctataaatcattgtttccatgttaagacttctgttctaattcattcattcaacaaacatttgttgagggccagatactgtgctggtcaactggggatactgtcctgaacaatatgcacacagttcttgcccctgcagagcttaaagaccattaaaggatacagaaaattaagcaattatcctatagtaggattcgtggtgtgatggggaaaatacaaggtactgtatttgggagtcatgtaactccagttttgggagtgagagagggtctggggaagaactgttccagataaaaagatgtgtaaccaagatagcatagcacattctgacaactaaagcaatttactctaacttaagcatgaagtacaaaagtatagttcatttattgagggaatgttaagctattaaaatgatactttttggtacttttaaaaaaatgaatacttaaacaccgttttaaaattttgtgttaagagctcctaataagaacttggtaaacatgttgacaatttaactcacagtctaaactttaaaagtagtagttacccttttatttctgtgccatgagacaaattcttctgcagttttatagttgtacaagatgttttgactcactctcaattgccatattcttaaagaaaccctgaaaagctgctcaagctggtcagttttctgtaaaaccaagtaaatgaaattttctctcccctcagaaaggaagttctacagcctagagtaccatctattgtgttttataagtccagatttaggctttgaaagatcataccaaaacttcagatcgacttaaacagcaactatgaattaggtttatgggaaatgtagtaatacaggggaaagaatatcggtaatggagtgaaatggtcctgggttcaaatccagtcttcctcacatattgtgctctttaagcttcaaatgagatgtataactgccttttccttaccagtaaaatgaggacaataattatgacaccatagggtggttgtgaggattagcaaatgttatcttgtatttcaggtgcctttcagggaaaggcttggcacatagtaaacacatcctgtaaatatcagtttccccttgttagcccctgtagcatcctgacctaccaagtctataggccttgcatatcactgtgcaccagagatacctcttttacatctgggctctgttttctctctatcaatcctatcctccaccctctttaaatcagaccccaggtttaaccttcccacagtcagtataaatcatttttcagaactttcagctttccatcaagtgaaagttgattttagttacccagtaatgtacagaaaccttcaataatgttaagcttcaaaggcttctggtacgtttgtctaccacagaaccaccacaatatatatgcttgttcttaggtttattgctttttattttaatggggtttggttgtttgggttttattttggttttggcttattttaaagtcaagtttagtttttactcaggtaaaggcctgaagtttcttgggcgtggaagaaaaagcttgggaagcctattaattcatggtgcagttcagaattgtttaaataagctctgtgccactgagcttaatctgaaagtatagtgacatgtgaaggatgcacatatgaatgaaaaactaaaataattgtaggctcttctataaatgaaaaaaaaagtgattggattgtgtctacctttttcctcaaagtaatctaacctaaacggtcatggtgttaaaactttttatggtgctaatcagaaaaattgatgttgcaagaaatgaggtctcaaaaatgggaatgtgcctcctcactctgcgtcacatgggaaataaatcctgaggccaggatcaccagtggggtagagtcatcttcatagtctcctgttatcctccccctgaacaaagaacaaagtgagactgggaagctagtgttggggaaacatttctgggataaactgtggagtggagaagacaatggagggcaaggcaaaaacccacagacttgtttacccatagaggccccactcacctccatgctgacagaattccagaaaaattccttccctatctcccaaacacctgaaattctgaaatattgccctactatttagcactttaagcttcagtgaggtgctgtaatggagagatagcttcatagtttagcctatagccaactttgcctcaagacctccagataagatgaagtggcttttacctcatgtgagtctcaaacgctggagagagagttccacaaccacccctgccctttctcccatattacgctcattagaaagtctactcagggctgggtgccgtggctcatgcctgtaatcccagcactttggaggctgaggcgggtggatcatctgaggtcaggagttcgagaccagcctggccaacatggggaaaccctgtctctactgaaaatataaaaattagccgggcatggtggcgtgcacctgtaattccagctactcgggaggctgaggcaggagaatcacttgaaccccagaggcagaggctacagtgagccaagattgcaccactgcactccaacctgggtgatagaataagactcttgtctcaaggaaagaaaaaatgtctactcagtagagcagtgattggcaaagtgtgagaccccagaccggcaatattggcatctcctggagacttgtttaaaatgctgattactattttcaagaactttctaccttcatttttacttattttttgttttcaagggctcatggaatcagtcttccatgtgacagctttttaaatattttaagacaaaaactgagtcttctaatcttaattgtgttctagcagtagtcattgtaattatagttatttttataatttatttgtttagtgtatatctgtcactctagaatgtaggctccctgagataaaaactataattcttttgatggcagttacattcccaactcttagcacaagatctaggacatagtatgaactcaataaatttttgttgaatgaaagaaaaatgcaaatttataagccttttcagagacctactgaatcaaactctgagagtggagcccagcaatctgttgtaaaagctgtttgagtaatactgttgcactctaagcttgagaaccactgcaatagaactaagagaacattcatttcaccttcaggcctattctctgacatcacttgaaaaatcctctctgccttctgtaacccttaaccaacaacagtgccctgtccatgtaggggaaacttctgggctgtattacaagtgggtaggaaggtataaggatatcatagtgggaagtattgcttagaatgccagtacttgtttgctgtggaaggtttctaggaatggattgcttagtctaaagtaagcactaccaccatccacgttttcactcaacacagtgccttttaattggtgactaacatcaaaacaccttgacaaaccgttaaaacagtatcagaaaccatgtgtacatccctgtgcctcttagattgctaagttctgctctcattcttacttcaaaggacactttttaaagaattgttaatgcttttttataatacaagtcatagccctgttggcaaaaagaaaaaagattcagtgagaaagaaaaatcactgataattccatcatccagggagaaccactgttatttaagtatatggccttccagacttttctgtgcatttatagatatttttaatgggatcaaagtgtacatactgttttgtaactttttttcactcaacaataccatgactgtcttgctgtgtcaagtatatttctacatctcttaatggctgcatgatattccatcatatgtgccacattttattaaactactgttcaatttttaggtttccaactttcattaataaagtgctttaaagaacattgtgttgctaaatctcgtatgtgttcatattttctcacgataaatgctttgaagtggaattgctagtgaagagggtatgccagtttttaaaagcttttgttactgccatgttgccccctagaaaactctaagcaatttatacttttatcagcagtatatgagagaatactgtttcttttggagggcttccaactagaatgttcttggagtctttggtttaaaaataaagggtaagagggagataaaaagatcctgcaaaattaaagtccagtcttaaatgtttcttaaattatgacattcatttactgttcttccctctttatcatatattttgtattactttggaaatgtttgtagtactagttgcctcttgcttttattcagtctcctggaagcattattacctttctgacatttgcaaagacagtagaagaacaatcttaagtagacttgaatcttgaggtatctttgatgcaggtgtactgttgatggatttagggaatgtagccgtagtggggaaagtaggattttggaaggtaggcagaacagtggctctcccacttgttttcctggccttttgtcagtcattaaacctctcccactttgagcctgcttactcatctctagaattagaatactataaacctaccttgtagcattgaaaggattaaccatattatgaagagtggctggcacctagtggataatattatggcttactaggatttatagatgagcatgttagagcatttgctattgtcaacctctggagtaaatatcactgccattgccaaagaaaatcttccttgaaagctgtcacagtgaatgagcattgtcttgtatatccttatacctacgcaatcctcattcttatggaatctggctcattttttccagtcaggtattcaagtaatgtaaccagataaaactctcagacaagatgtacagtggaagatttcaacattaccatgcttaacaacagtgtacgaaagtggtaatcacagtctttttaagtggtaaaccttgccaaactgccccttctgatactcccccacccttggcccacagctgatctcagtgcttcggggggctgtacctctatgtgagagcctgacataggacaggtgactcagctcagtaacagtattgttaattagctaagtctcagtagcatctccaggactggagatcacagaattgattcatccatagtgagagaaatgtatttaaagaagtatctgaattataaaagttgctcagcagtattcaaaaagatagatgtcccagggttgagctgcagtcatcatagcaaaaccccacagaccaacccattttaatatttggatgaaccataataaatttaaattaacatctgttgattataactcaaactgctcaaccaataaagaatgagtagtaagaggacttaagggggttgtaagtaaggaaggctatacaggaagcttcaggcttacgagaacaagacgaatagctaattggaggtgagatggactaccattataagcaacttttctgagagtctgatgcaccagggtttagggcacatgtttctgctcagtctcatgttctagtaaagttctctgaagcttgagctgtagtaggcagttcccaacaaccaggcattttgatacctgcttgccttttctatgtattttcttgtgttcagcttcacctgaagatcctcatttgtcaagaataagctcaaactctcattcctccatgaagagattctggattccgcaggcagaattgcatacacacacacaactatcacatgcattttgccttggatcacaattacttgccaacttgaaggacaggattttgtgtcccctgacacctagcacacctaaagctgagttttggcaagctcatagcttgcttgcagcattgtatttgcaaaaaaatctattaatgattcaaatttggtgctattatgtgtatttgccctaagaaacattggctgtttaggacacca

SEQ ID NO: 7 - DNA, 6382 bp, Artificial Sequence
  Description of Artificial Sequence Synthetic polynucleotide
gcgcttgtttttcccttgggacccgggtccacacggcggggtcgcccgtccatctccggctcgcccgcggggcccatcgtcgacgttagcggccgttctccgagccgactgacccatccttggcgctgccgccgcgcgcttgttctcctccctcgccccgccttcatcctccccgttcacggaaacgacagctgcggctgcggggctggcgccgcctccctccacctaccacgtctgccctcgccgctctagccctgcgccccagcccggccgcggcacctccgcctcgccgccgctaggtcggccggctccgcccggctgccgcctaggatgaatatcatggacttcaacgtgaagaagctggcggccgacgcaggcaccttcctcagtcgcgccgtgcagttcacagaagaaaagcttggccaggctgagaagacagaattggatgctcacttagagaacctccttagcaaagctgaatgtaccaaaatatggacagaaaaaataatgaaacaaactgaagtgttattgcagccaaatccaaatgccaggatagaagaatttgtttatgagaaactggatagaaaagctccaagtcgtataaacaacccagaacttttgggacaatatatgattgatgcagggactgagtttggcccaggaacagcttatggtaatgcccttattaaatgtggagaaacccaaaaaagaattggaacagcagacagagaactgattcaaacgtcagccttaaattttcttactcctttaagaaactttatagaaggagattacaaaacaattgctaaagaaaggaaactattgcaaaataagagactggatttggatgctgcaaaaacgagactaaaaaaggcaaaagctgcagaaactagaaattcatctgaacaggaattaagaataactcaaagtgaatttgatcgtcaagcagagattaccagacttctgctagagggaatcagcagtacacatgcccatcaccttcgctgtctgaatgactttgtagaagcccagatgacttactatgcacagtgttaccagtatatgttggacctccagaaacaactgggaagttttccatccaattatcttagtaacaacaatcagacttctgtgacacctgtaccatcagttttaccaaatgcgattggttcttctgccatggcttcaacaagtggcctagtaatcacctctccttccaacctcagtgaccttaaggagtgtagtggcagcagaaaggccagggttctctatgattatgatgcagcaaacagtactgaattatcacttctggcagatgaggtgatcactgtgttcagtgttgttggaatggattcagactggctaatgggggaaaggggaaaccagaagggcaaggtgccaattacctacttagaactgctcaattaagtaggtggactatggaaaggttgcccatcatgactttgtatttatatacaattaactctaaataaagcaggttaagtatcttccatgttaatgtgttaagagactgaaaataccagccatcagaaactggcctttctgccaataaagttgcatggtaaatatttcattacagaatttatgttagagctttcatgccaagaatgttttcttacaaaattctctttttattgaggtttcactaataagcagcttctacttttgagcctcaacttaaagcagaactgttttctactggatttttcattaacagcaagctttttcttttatgtaaaataaatctattgtgaattgatatcagcgactcatttattaggtataaataatagccaaagaataaaattaaaatttattttaaactttcggtcttaaaaagagctgtaggatataaacctcttttgtgaaaagtagaattttctgaatgctttcatagacaaaaacgcatttaaactatgtttacctggttttccaacagaaatcaaatgattttaatattatgttttgatgggttattttcagagttgtttggttttttaaacactactgaatggtttcttttaaatttaagaacaactagttcttgaaaagcaataccgtatttctctgataaatttgtagttaaggttctagaaaatctaggacaaatttacttcattaaacataaagtttttaagattattcagttggcacaatttaatgcataattgggattggatccatttactgtactgtggtacagtaaagagaatgtgttcttattgaacagcagatttgtatacaaatacggaataatgcaactacattataaattgtatacgaacaaaataaattgggtcactctgagcttactattaaaacattgaaaatttattaatgaacacaggctatcttgatgaggatctctgcttatgaaacacatactaatgaaattattagtgacacattggaagctagaaaattgctgttggggccattgctataaatcattgtttccatgttaagacttctgttctaattcattcattcaacaaacatttgttgagggccagatactgtgctggtcaactggggatactgtcctgaacaatatgcacacagttcttgcccctgcagagcttaaagaccattaaaggatacagaaaattaagcaattatcctatagtaggattcgtggtgtgatggggaaaatacaaggtactgtatttgggagtcatgtaactccagttttgggagtgagagagggtctggggaagaactgttccagataaaaagatgtgtaaccaagatagcatagcacattctgacaactaaagcaatttactctaacttaagcatgaagtacaaaagtatagttcatttattgagggaatgttaagctattaaaatgatactttttggtacttttaaaaaaatgaatacttaaacaccgttttaaaattttgtgttaagagctcctaataagaacttggtaaacatgttgacaatttaactcacagtctaaactttaaaagtagtagttacccttttatttctgtgccatgagacaaattcttctgcagttttatagttgtacaagatgttttgactcactctcaattgccatattcttaaagaaaccctgaaaagctgctcaagctggtcagttttctgtaaaaccaagtaaatgaaattttctctcccctcagaaaggaagttctacagcctagagtaccatctattgtgttttataagtccagatttaggctttgaaagatcataccaaaacttcagatcgacttaaacagcaactatgaattaggtttatgggaaatgtagtaatacaggggaaagaatatcggtaatggagtgaaatggtcctgggttcaaatccagtcttcctcacatattgtgctctttaagcttcaaatgagatgtataactgccttttccttaccagtaaaatgaggacaataattatgacaccatagggtggttgtgaggattagcaaatgttatcttgtatttcaggtgcctttcagggaaaggcttggcacatagtaaacacatcctgtaaatatcagtttccccttgttagcccctgtagcatcctgacctaccaagtctataggccttgcatatcactgtgcaccagagatacctcttttacatctgggctctgttttctctctatcaatcctatcctccaccctctttaaatcagaccccaggtttaaccttcccacagtcagtataaatcatttttcagaactttcagctttccatcaagtgaaagttgattttagttacccagtaatgtacagaaaccttcaataatgttaagcttcaaaggcttctggtacgtttgtctaccacagaaccaccacaatatatatgcttgttcttaggtttattgctttttattttaatggggtttggttgtttgggttttattttggttttggcttattttaaagtcaagtttagtttttactcaggtaaaggcctgaagtttcttgggcgtggaagaaaaagcttgggaagcctattaattcatggtgcagttcagaattgtttaaataagctctgtgccactgagcttaatctgaaagtatagtgacatgtgaaggatgcacatatgaatgaaaaactaaaataattgtaggctcttctataaatgaaaaaaaaagtgattggattgtgtctacctttttcctcaaagtaatctaacctaaacggtcatggtgttaaaactttttatggtgctaatcagaaaaattgatgttgcaagaaatgaggtctcaaaaatgggaatgtgcctcctcactctgcgtcacatgggaaataaatcctgaggccaggatcaccagtggggtagagtcatcttcatagtctcctgttatcctccccctgaacaaagaacaaagtgagactgggaagctagtgttggggaaacatttctgggataaactgtggagtggagaagacaatggagggcaaggcaaaaacccacagacttgtttacccatagaggccccactcacctccatgctgacagaattccagaaaaattccttccctatctcccaaacacctgaaattctgaaatattgccctactatttagcactttaagcttcagtgaggtgctgtaatggagagatagcttcatagtttagcctatagccaactttgcctcaagacctccagataagatgaagtggcttttacctcatgtgagtctcaaacgctggagagagagttccacaaccacccctgccctttctcccatattacgctcattagaaagtctactcagggctgggtgccgtggctcatgcctgtaatcccagcactttggaggctgaggcgggtggatcatctgaggtcaggagttcgagaccagcctggccaacatggggaaaccctgtctctactgaaaatataaaaattagccgggcatggtggcgtgcacctgtaattccagctactcgggaggctgaggcaggagaatcacttgaaccccagaggcagaggctacagtgagccaagattgcaccactgcactccaacctgggtgatagaataagactcttgtctcaaggaaagaaaaaatgtctactcagtagagcagtgattggcaaagtgtgagaccccagaccggcaatattggcatctcctggagacttgtttaaaatgctgattactattttcaagaactttctaccttcatttttacttattttttgttttcaagggctcatggaatcagtcttccatgtgacagctttttaaatattttaagacaaaaactgagtcttctaatcttaattgtgttctagcagtagtcattgtaattatagttatttttataatttatttgtttagtgtatatctgtcactctagaatgtaggctccctgagataaaaactataattcttttgatggcagttacattcccaactcttagcacaagatctaggacatagtatgaactcaataaatttttgttgaatgaaagaaaaatgcaaatttataagccttttcagagacctactgaatcaaactctgagagtggagcccagcaatctgttgtaaaagctgtttgagtaatactgttgcactctaagcttgagaaccactgcaatagaactaagagaacattcatttcaccttcaggcctattctctgacatcacttgaaaaatcctctctgccttctgtaacccttaaccaacaacagtgccctgtccatgtaggggaaacttctgggctgtattacaagtgggtaggaaggtataaggatatcatagtgggaagtattgcttagaatgccagtacttgtttgctgtggaaggtttctaggaatggattgcttagtctaaagtaagcactaccaccatccacgttttcactcaacacagtgccttttaattggtgactaacatcaaaacaccttgacaaaccgttaaaacagtatcagaaaccatgtgtacatccctgtgcctcttagattgctaagttctgctctcattcttacttcaaaggacactttttaaagaattgttaatgcttttttataatacaagtcatagccctgttggcaaaaagaaaaaagattcagtgagaaagaaaaatcactgataattccatcatccagggagaaccactgttatttaagtatatggccttccagacttttctgtgcatttatagatatttttaatgggatcaaagtgtacatactgttttgtaactttttttcactcaacaataccatgactgtcttgctgtgtcaagtatatttctacatctcttaatggctgcatgatattccatcatatgtgccacattttattaaactactgttcaatttttaggtttccaactttcattaataaagtgctttaaagaacaaaaaa

SEQ ID NO: 8 - DNA, 6469 bp, Artificial Sequence
  Description of Artificial Sequence Synthetic polynucleotide
gcgcttgtttttcccttgggacccgggtccacacggcggggtcgcccgtccatctccggctcgcccgcggggcccatcgtcgacgttagcggccgttctccgagccgactgacccatccttggcgctgccgccgcgcgcttgttctcctccctcgccccgccttcatcctccccgttcacggaaacgacagctgcggctgcggggctggcgccgcctccctccacctaccacgtctgccctcgccgctctagccctgcgccccagcccggccgcggcacctccgcctcgccgccgctaggtcggccggctccgcccggctgccgcctaggatgaatatcatggacttcaacgtgaagaagctggcggccgacgcaggcaccttcctcagtcgcgccgtgcagttcacagaagaaaagcttggccaggctgagaagacagaattggatgctcacttagagaacctccttagcaaagctgaatgtaccaaaatatggacagaaaaaataatgaaacaaactgaagtgttattgcagccaaatccaaatgccaggatagaagaatttgtttatgagaaactggatagaaaagctccaagtcgtataaacaacccagaacttttgggacaatatatgattgatgcagggactgagtttggcccaggaacagcttatggtaatgcccttattaaatgtggagaaacccaaaaaagaattggaacagcagacagagaactgattcaaacgtcagccttaaattttcttactcctttaagaaactttatagaaggagattacaaaacaattgctaaagaaaggaaactattgcaaaataagagactggatttggatgctgcaaaaacgagactaaaaaaggcaaaagctgcagaaactagaaattcacaactaaactcagctcgccttgaaggagataacattatggtaaatttctcttacatgctcaacttcctgcatgtaaaatggctgaagtctgaacaggaattaagaataactcaaagtgaatttgatcgtcaagcagagattaccagacttctgctagagggaatcagcagtacacatgcccatcaccttcgctgtctgaatgactttgtagaagcccagatgacttactatgcacagtgttaccagtatatgttggacctccagaaacaactgggaagttttccatccaattatcttagtaacaacaatcagacttctgtgacacctgtaccatcagttttaccaaatgcgattggttcttctgccatggcttcaacaagtggcctagtaatcacctctccttccaacctcagtgaccttaaggagtgtagtggcagcagaaaggccagggttctctatgattatgatgcagcaaacagtactgaattatcacttctggcagatgaggtgatcactgtgttcagtgttgttggaatggattcagactggctaatgggggaaaggggaaaccagaagggcaaggtgccaattacctacttagaactgctcaattaagtaggtggactatggaaaggttgcccatcatgactttgtatttatatacaattaactctaaataaagcaggttaagtatcttccatgttaatgtgttaagagactgaaaataccagccatcagaaactggcctttctgccaataaagttgcatggtaaatatttcattacagaatttatgttagagctttcatgccaagaatgttttcttacaaaattctctttttattgaggtttcactaataagcagcttctacttttgagcctcaacttaaagcagaactgttttctactggatttttcattaacagcaagctttttcttttatgtaaaataaatctattgtgaattgatatcagcgactcatttattaggtataaataatagccaaagaataaaattaaaatttattttaaactttcggtcttaaaaagagctgtaggatataaacctcttttgtgaaaagtagaattttctgaatgctttcatagacaaaaacgcatttaaactatgtttacctggttttccaacagaaatcaaatgattttaatattatgttttgatgggttattttcagagttgtttggttttttaaacactactgaatggtttcttttaaatttaagaacaactagttcttgaaaagcaataccgtatttctctgataaatttgtagttaaggttctagaaaatctaggacaaatttacttcattaaacataaagtttttaagattattcagttggcacaatttaatgcataattgggattggatccatttactgtactgtggtacagtaaagagaatgtgttcttattgaacagcagatttgtatacaaatacggaataatgcaactacattataaattgtatacgaacaaaataaattgggtcactctgagcttactattaaaacattgaaaatttattaatgaacacaggctatcttgatgaggatctctgcttatgaaacacatactaatgaaattattagtgacacattggaagctagaaaattgctgttggggccattgctataaatcattgtttccatgttaagacttctgttctaattcattcattcaacaaacatttgttgagggccagatactgtgctggtcaactggggatactgtcctgaacaatatgcacacagttcttgcccctgcagagcttaaagaccattaaaggatacagaaaattaagcaattatcctatagtaggattcgtggtgtgatggggaaaatacaaggtactgtatttgggagtcatgtaactccagttttgggagtgagagagggtctggggaagaactgttccagataaaaagatgtgtaaccaagatagcatagcacattctgacaactaaagcaatttactctaacttaagcatgaagtacaaaagtatagttcatttattgagggaatgttaagctattaaaatgatactttttggtacttttaaaaaaatgaatacttaaacaccgttttaaaattttgtgttaagagctcctaataagaacttggtaaacatgttgacaatttaactcacagtctaaactttaaaagtagtagttacccttttatttctgtgccatgagacaaattcttctgcagttttatagttgtacaagatgttttgactcactctcaattgccatattcttaaagaaaccctgaaaagctgctcaagctggtcagttttctgtaaaaccaagtaaatgaaattttctctcccctcagaaaggaagttctacagcctagagtaccatctattgtgttttataagtccagatttaggctttgaaagatcataccaaaacttcagatcgacttaaacagcaactatgaattaggtttatgggaaatgtagtaatacaggggaaagaatatcggtaatggagtgaaatggtcctgggttcaaatccagtcttcctcacatattgtgctctttaagcttcaaatgagatgtataactgccttttccttaccagtaaaatgaggacaataattatgacaccatagggtggttgtgaggattagcaaatgttatcttgtatttcaggtgcctttcagggaaaggcttggcacatagtaaacacatcctgtaaatatcagtttccccttgttagcccctgtagcatcctgacctaccaagtctataggccttgcatatcactgtgcaccagagatacctcttttacatctgggctctgttttctctctatcaatcctatcctccaccctctttaaatcagaccccaggtttaaccttcccacagtcagtataaatcatttttcagaactttcagctttccatcaagtgaaagttgattttagttacccagtaatgtacagaaaccttcaataatgttaagcttcaaaggcttctggtacgtttgtctaccacagaaccaccacaatatatatgcttgttcttaggtttattgctttttattttaatggggtttggttgtttgggttttattttggttttggcttattttaaagtcaagtttagtttttactcaggtaaaggcctgaagtttcttgggcgtggaagaaaaagcttgggaagcctattaattcatggtgcagttcagaattgtttaaataagctctgtgccactgagcttaatctgaaagtatagtgacatgtgaaggatgcacatatgaatgaaaaactaaaataattgtaggctcttctataaatgaaaaaaaaagtgattggattgtgtctacctttttcctcaaagtaatctaacctaaacggtcatggtgttaaaactttttatggtgctaatcagaaaaattgatgttgcaagaaatgaggtctcaaaaatgggaatgtgcctcctcactctgcgtcacatgggaaataaatcctgaggccaggatcaccagtggggtagagtcatcttcatagtctcctgttatcctccccctgaacaaagaacaaagtgagactgggaagctagtgttggggaaacatttctgggataaactgtggagtggagaagacaatggagggcaaggcaaaaacccacagacttgtttacccatagaggccccactcacctccatgctgacagaattccagaaaaattccttccctatctcccaaacacctgaaattctgaaatattgccctactatttagcactttaagcttcagtgaggtgctgtaatggagagatagcttcatagtttagcctatagccaactttgcctcaagacctccagataagatgaagtggcttttacctcatgtgagtctcaaacgctggagagagagttccacaaccacccctgccctttctcccatattacgctcattagaaagtctactcagggctgggtgccgtggctcatgcctgtaatcccagcactttggaggctgaggcgggtggatcatctgaggtcaggagttcgagaccagcctggccaacatggggaaaccctgtctctactgaaaatataaaaattagccgggcatggtggcgtgcacctgtaattccagctactcgggaggctgaggcaggagaatcacttgaaccccagaggcagaggctacagtgagccaagattgcaccactgcactccaacctgggtgatagaataagactcttgtctcaaggaaagaaaaaatgtctactcagtagagcagtgattggcaaagtgtgagaccccagaccggcaatattggcatctcctggagacttgtttaaaatgctgattactattttcaagaactttctaccttcatttttacttattttttgttttcaagggctcatggaatcagtcttccatgtgacagctttttaaatattttaagacaaaaactgagtcttctaatcttaattgtgttctagcagtagtcattgtaattatagttatttttataatttatttgtttagtgtatatctgtcactctagaatgtaggctccctgagataaaaactataattcttttgatggcagttacattcccaactcttagcacaagatctaggacatagtatgaactcaataaatttttgttgaatgaaagaaaaatgcaaatttataagccttttcagagacctactgaatcaaactctgagagtggagcccagcaatctgttgtaaaagctgtttgagtaatactgttgcactctaagcttgagaaccactgcaatagaactaagagaacattcatttcaccttcaggcctattctctgacatcacttgaaaaatcctctctgccttctgtaacccttaaccaacaacagtgccctgtccatgtaggggaaacttctgggctgtattacaagtgggtaggaaggtataaggatatcatagtgggaagtattgcttagaatgccagtacttgtttgctgtggaaggtttctaggaatggattgcttagtctaaagtaagcactaccaccatccacgttttcactcaacacagtgccttttaattggtgactaacatcaaaacaccttgacaaaccgttaaaacagtatcagaaaccatgtgtacatccctgtgcctcttagattgctaagttctgctctcattcttacttcaaaggacactttttaaagaattgttaatgcttttttataatacaagtcatagccctgttggcaaaaagaaaaaagattcagtgagaaagaaaaatcactgataattccatcatccagggagaaccactgttatttaagtatatggccttccagacttttctgtgcatttatagatatttttaatgggatcaaagtgtacatactgttttgtaactttttttcactcaacaataccatgactgtcttgctgtgtcaagtatatttctacatctcttaatggctgcatgatattccatcatatgtgccacattttattaaactactgttcaatttttaggtttccaactttcattaataaagtgctttaaagaacaaaaaa

SEQ ID NO: 9 - DNA, 6445 bp, Artificial Sequence
  Description of Artificial Sequence Synthetic polynucleotide
gcgcttgtttttcccttgggacccgggtccacacggcggggtcgcccgtccatctccggctcgcccgcggggcccatcgtcgacgttagcggccgttctccgagccgactgacccatccttggcgctgccgccgcgcgcttgttctcctccctcgccccgccttcatcctccccgttcacggaaacgacagctgcggctgcggggctggcgccgcctccctccacctaccacgtctgccctcgccgctctagccctgcgccccagcccggccgcggcacctccgcctcgccgccgctaggtcggccggctccgcccggctgccgcctaggatgaatatcatggacttcaacgtgaagaagctggcggccgacgcaggcaccttcctcagtcgcgccgtgcagttcacagaagaaaagcttggccaggctgagaagacagaattggatgctcacttagagaacctccttagcaaagctgaatgtaccaaaatatggacagaaaaaataatgaaacaaactgaagtgttattgcagccaaatccaaatgccaggatagaagaatttgtttatgagaaactggatagaaaagctccaagtcgtataaacaacccagaacttttgggacaatatatgattgatgcagggactgagtttggcccaggaacagcttatggtaatgcccttattaaatgtggagaaacccaaaaaagaattggaacagcagacagagaactgattcaaacgtcagccttaaattttcttactcctttaagaaactttatagaaggagattacaaaacaattgctaaagaaaggaaactattgcaaaataagagactggatttggatgctgcaaaaacgagactaaaaaaggcaaaagctgcagaaactagaaattcacaactaaactcagctcgccttgaaggagataacattatgatttgggcagaggaggtgacaaaatctgaacaggaattaagaataactcaaagtgaatttgatcgtcaagcagagattaccagacttctgctagagggaatcagcagtacacatgcccatcaccttcgctgtctgaatgactttgtagaagcccagatgacttactatgcacagtgttaccagtatatgttggacctccagaaacaactgggaagttttccatccaattatcttagtaacaacaatcagacttctgtgacacctgtaccatcagttttaccaaatgcgattggttcttctgccatggcttcaacaagtggcctagtaatcacctctccttccaacctcagtgaccttaaggagtgtagtggcagcagaaaggccagggttctctatgattatgatgcagcaaacagtactgaattatcacttctggcagatgaggtgatcactgtgttcagtgttgttggaatggattcagactggctaatgggggaaaggggaaaccagaagggcaaggtgccaattacctacttagaactgctcaattaagtaggtggactatggaaaggttgcccatcatgactttgtatttatatacaattaactctaaataaagcaggttaagtatcttccatgttaatgtgttaagagactgaaaataccagccatcagaaactggcctttctgccaataaagttgcatggtaaatatttcattacagaatttatgttagagctttcatgccaagaatgttttcttacaaaattctctttttattgaggtttcactaataagcagcttctacttttgagcctcaacttaaagcagaactgttttctactggatttttcattaacagcaagctttttcttttatgtaaaataaatctattgtgaattgatatcagcgactcatttattaggtataaataatagccaaagaataaaattaaaatttattttaaactttcggtcttaaaaagagctgtaggatataaacctcttttgtgaaaagtagaattttctgaatgctttcatagacaaaaacgcatttaaactatgtttacctggttttccaacagaaatcaaatgattttaatattatgttttgatgggttattttcagagttgtttggttttttaaacactactgaatggtttcttttaaatttaagaacaactagttcttgaaaagcaataccgtatttctctgataaatttgtagttaaggttctagaaaatctaggacaaatttacttcattaaacataaagtttttaagattattcagttggcacaatttaatgcataattgggattggatccatttactgtactgtggtacagtaaagagaatgtgttcttattgaacagcagatttgtatacaaatacggaataatgcaactacattataaattgtatacgaacaaaataaattgggtcactctgagcttactattaaaacattgaaaatttattaatgaacacaggctatcttgatgaggatctctgcttatgaaacacatactaatgaaattattagtgacacattggaagctagaaaattgctgttggggccattgctataaatcattgtttccatgttaagacttctgttctaattcattcattcaacaaacatttgttgagggccagatactgtgctggtcaactggggatactgtcctgaacaatatgcacacagttcttgcccctgcagagcttaaagaccattaaaggatacagaaaattaagcaattatcctatagtaggattcgtggtgtgatggggaaaatacaaggtactgtatttgggagtcatgtaactccagttttgggagtgagagagggtctggggaagaactgttccagataaaaagatgtgtaaccaagatagcatagcacattctgacaactaaagcaatttactctaacttaagcatgaagtacaaaagtatagttcatttattgagggaatgttaagctattaaaatgatactttttggtacttttaaaaaaatgaatacttaaacaccgttttaaaattttgtgttaagagctcctaataagaacttggtaaacatgttgacaatttaactcacagtctaaactttaaaagtagtagttacccttttatttctgtgccatgagacaaattcttctgcagttttatagttgtacaagatgttttgactcactctcaattgccatattcttaaagaaaccctgaaaagctgctcaagctggtcagttttctgtaaaaccaagtaaatgaaattttctctcccctcagaaaggaagttctacagcctagagtaccatctattgtgttttataagtccagatttaggctttgaaagatcataccaaaacttcagatcgacttaaacagcaactatgaattaggtttatgggaaatgtagtaatacaggggaaagaatatcggtaatggagtgaaatggtcctgggttcaaatccagtcttcctcacatattgtgctctttaagcttcaaatgagatgtataactgccttttccttaccagtaaaatgaggacaataattatgacaccatagggtggttgtgaggattagcaaatgttatcttgtatttcaggtgcctttcagggaaaggcttggcacatagtaaacacatcctgtaaatatcagtttccccttgttagcccctgtagcatcctgacctaccaagtctataggccttgcatatcactgtgcaccagagatacctcttttacatctgggctctgttttctctctatcaatcctatcctccaccctctttaaatcagaccccaggtttaaccttcccacagtcagtataaatcatttttcagaactttcagctttccatcaagtgaaagttgattttagttacccagtaatgtacagaaaccttcaataatgttaagcttcaaaggcttctggtacgtttgtctaccacagaaccaccacaatatatatgcttgttcttaggtttattgctttttattttaatggggtttggttgtttgggttttattttggttttggcttattttaaagtcaagtttagtttttactcaggtaaaggcctgaagtttcttgggcgtggaagaaaaagcttgggaagcctattaattcatggtgcagttcagaattgtttaaataagctctgtgccactgagcttaatctgaaagtatagtgacatgtgaaggatgcacatatgaatgaaaaactaaaataattgtaggctcttctataaatgaaaaaaaaagtgattggattgtgtctacctttttcctcaaagtaatctaacctaaacggtcatggtgttaaaactttttatggtgctaatcagaaaaattgatgttgcaagaaatgaggtctcaaaaatgggaatgtgcctcctcactctgcgtcacatgggaaataaatcctgaggccaggatcaccagtggggtagagtcatcttcatagtctcctgttatcctccccctgaacaaagaacaaagtgagactgggaagctagtgttggggaaacatttctgggataaactgtggagtggagaagacaatggagggcaaggcaaaaacccacagacttgtttacccatagaggccccactcacctccatgctgacagaattccagaaaaattccttccctatctcccaaacacctgaaattctgaaatattgccctactatttagcactttaagcttcagtgaggtgctgtaatggagagatagcttcatagtttagcctatagccaactttgcctcaagacctccagataagatgaagtggcttttacctcatgtgagtctcaaacgctggagagagagttccacaaccacccctgccctttctcccatattacgctcattagaaagtctactcagggctgggtgccgtggctcatgcctgtaatcccagcactttggaggctgaggcgggtggatcatctgaggtcaggagttcgagaccagcctggccaacatggggaaaccctgtctctactgaaaatataaaaattagccgggcatggtggcgtgcacctgtaattccagctactcgggaggctgaggcaggagaatcacttgaaccccagaggcagaggctacagtgagccaagattgcaccactgcactccaacctgggtgatagaataagactcttgtctcaaggaaagaaaaaatgtctactcagtagagcagtgattggcaaagtgtgagaccccagaccggcaatattggcatctcctggagacttgtttaaaatgctgattactattttcaagaactttctaccttcatttttacttattttttgttttcaagggctcatggaatcagtcttccatgtgacagctttttaaatattttaagacaaaaactgagtcttctaatcttaattgtgttctagcagtagtcattgtaattatagttatttttataatttatttgtttagtgtatatctgtcactctagaatgtaggctccctgagataaaaactataattcttttgatggcagttacattcccaactcttagcacaagatctaggacatagtatgaactcaataaatttttgttgaatgaaagaaaaatgcaaatttataagccttttcagagacctactgaatcaaactctgagagtggagcccagcaatctgttgtaaaagctgtttgagtaatactgttgcactctaagcttgagaaccactgcaatagaactaagagaacattcatttcaccttcaggcctattctctgacatcacttgaaaaatcctctctgccttctgtaacccttaaccaacaacagtgccctgtccatgtaggggaaacttctgggctgtattacaagtgggtaggaaggtataaggatatcatagtgggaagtattgcttagaatgccagtacttgtttgctgtggaaggtttctaggaatggattgcttagtctaaagtaagcactaccaccatccacgttttcactcaacacagtgccttttaattggtgactaacatcaaaacaccttgacaaaccgttaaaacagtatcagaaaccatgtgtacatccctgtgcctcttagattgctaagttctgctctcattcttacttcaaaggacactttttaaagaattgttaatgcttttttataatacaagtcatagccctgttggcaaaaagaaaaaagattcagtgagaaagaaaaatcactgataattccatcatccagggagaaccactgttatttaagtatatggccttccagacttttctgtgcatttatagatatttttaatgggatcaaagtgtacatactgttttgtaactttttttcactcaacaataccatgactgtcttgctgtgtcaagtatatttctacatctcttaatggctgcatgatattccatcatatgtgccacattttattaaactactgttcaatttttaggtttccaactttcattaataaagtgctttaaagaacaaaaaa

SEQ ID NO: 10 - DNA, 6240 bp, Homo sapiens
gcgcttgtttttcccttgggacccgggtccacacggcggggtcgcccgtccatctccggctcgcccgcggggcccatcgtcgacgttagcggccgttctccgagccgactgacccatccttggcgctgccgccgcgcgcttgttctcctccctcgccccgccttcatcctccccgttcacggaaacgacagctgcggctgcggggctggcgccgcctccctccacctaccacgtctgccctcgccgctctagccctgcgccccagcccggccgcggcacctccgcctcgccgccgctaggtcggccggctccgcccggctgccgcctaggatgaatatcatggacttcaacgtgaagaagctggcggccgacgcaggcaccttcctcagtcgcgccgtgcagatgccaggatagaagaatttgtttatgagaaactggatagaaaagctccaagtcgtataaacaacccagaacttttgggacaatatatgattgatgcagggactgagtttggcccaggaacagcttatggtaatgcccttattaaatgtggagaaacccaaaaaagaattggaacagcagacagagaactgattcaaacgtcagccttaaattttcttactcctttaagaaactttatagaaggagattacaaaacaattgctaaagaaaggaaactattgcaaaataagagactggatttggatgctgcaaaaacgagactaaaaaaggcaaaagctgcagaaactagaaattcatctgaacaggaattaagaataactcaaagtgaatttgatcgtcaagcagagattaccagacttctgctagagggaatcagcagtacacatgcccatcaccttcgctgtctgaatgactttgtagaagcccagatgacttactatgcacagtgttaccagtatatgttggacctccagaaacaactgggaagttttccatccaattatcttagtaacaacaatcagacttctgtgacacctgtaccatcagttttaccaaatgcgattggttcttctgccatggcttcaacaagtggcctagtaatcacctctccttccaacctcagtgaccttaaggagtgtagtggcagcagaaaggccagggttctctatgattatgatgcagcaaacagtactgaattatcacttctggcagatgaggtgatcactgtgttcagtgttgttggaatggattcagactggctaatgggggaaaggggaaaccagaagggcaaggtgccaattacctacttagaactgctcaattaagtaggtggactatggaaaggttgcccatcatgactttgtatttatatacaattaactctaaataaagcaggttaagtatcttccatgttaatgtgttaagagactgaaaataccagccatcagaaactggcctttctgccaataaagttgcatggtaaatatttcattacagaatttatgttagagctttcatgccaagaatgttttcttacaaaattctctttttattgaggtttcactaataagcagcttctacttttgagcctcaacttaaagcagaactgttttctactggatttttcattaacagcaagctttttcttttatgtaaaataaatctattgtgaattgatatcagcgactcatttattaggtataaataatagccaaagaataaaattaaaatttattttaaactttcggtcttaaaaagagctgtaggatataaacctcttttgtgaaaagtagaattttctgaatgctttcatagacaaaaacgcatttaaactatgtttacctggttttccaacagaaatcaaatgattttaatattatgttttgatgggttattttcagagttgtttggttttttaaacactactgaatggtttcttttaaatttaagaacaactagttcttgaaaagcaataccgtatttctctgataaatttgtagttaaggttctagaaaatctaggacaaatttacttcattaaacataaagtttttaagattattcagttggcacaatttaatgcataattgggattggatccatttactgtactgtggtacagtaaagagaatgtgttcttattgaacagcagatttgtatacaaatacggaataatgcaactacattataaattgtatacgaacaaaataaattgggtcactctgagcttactattaaaacattgaaaatttattaatgaacacaggctatcttgatgaggatctctgcttatgaaacacatactaatgaaattattagtgacacattggaagctagaaaattgctgttggggccattgctataaatcattgtttccatgttaagacttctgttctaattcattcattcaacaaacatttgttgagggccagatactgtgctggtcaactggggatactgtcctgaacaatatgcacacagttcttgcccctgcagagcttaaagaccattaaaggatacagaaaattaagcaattatcctatagtaggattcgtggtgtgatggggaaaatacaaggtactgtatttgggagtcatgtaactccagttttgggagtgagagagggtctggggaagaactgttccagataaaaagatgtgtaaccaagatagcatagcacattctgacaactaaagcaatttactctaacttaagcatgaagtacaaaagtatagttcatttattgagggaatgttaagctattaaaatgatactttttggtacttttaaaaaaatgaatacttaaacaccgttttaaaattttgtgttaagagctcctaataagaacttggtaaacatgttgacaatttaactcacagtctaaactttaaaagtagtagttacccttttatttctgtgccatgagacaaattcttctgcagttttatagttgtacaagatgttttgactcactctcaattgccatattcttaaagaaaccctgaaaagctgctcaagctggtcagttttctgtaaaaccaagtaaatgaaattttctctcccctcagaaaggaagttctacagcctagagtaccatctattgtgttttataagtccagatttaggctttgaaagatcataccaaaacttcagatcgacttaaacagcaactatgaattaggtttatgggaaatgtagtaatacaggggaaagaatatcggtaatggagtgaaatggtcctgggttcaaatccagtcttcctcacatattgtgctctttaagcttcaaatgagatgtataactgccttttccttaccagtaaaatgaggacaataattatgacaccatagggtggttgtgaggattagcaaatgttatcttgtatttcaggtgcctttcagggaaaggcttggcacatagtaaacacatcctgtaaatatcagtttccccttgttagcccctgtagcatcctgacctaccaagtctataggccttgcatatcactgtgcaccagagatacctcttttacatctgggctctgttttctctctatcaatcctatcctccaccctctttaaatcagaccccaggtttaaccttcccacagtcagtataaatcatttttcagaactttcagctttccatcaagtgaaagttgattttagttacccagtaatgtacagaaaccttcaataatgttaagcttcaaaggcttctggtacgtttgtctaccacagaaccaccacaatatatatgcttgttcttaggtttattgctttttattttaatggggtttggttgtttgggttttattttggttttggcttattttaaagtcaagtttagtttttactcaggtaaaggcctgaagtttcttgggcgtggaagaaaaagcttgggaagcctattaattcatggtgcagttcagaattgtttaaataagctctgtgccactgagcttaatctgaaagtatagtgacatgtgaaggatgcacatatgaatgaaaaactaaaataattgtaggctcttctataaatgaaaaaaaaagtgattggattgtgtctacctttttcctcaaagtaatctaacctaaacggtcatggtgttaaaactttttatggtgctaatcagaaaaattgatgttgcaagaaatgaggtctcaaaaatgggaatgtgcctcctcactctgcgtcacatgggaaataaatcctgaggccaggatcaccagtggggtagagtcatcttcatagtctcctgttatcctccccctgaacaaagaacaaagtgagactgggaagctagtgttggggaaacatttctgggataaactgtggagtggagaagacaatggagggcaaggcaaaaacccacagacttgtttacccatagaggccccactcacctccatgctgacagaattccagaaaaattccttccctatctcccaaacacctgaaattctgaaatattgccctactatttagcactttaagcttcagtgaggtgctgtaatggagagatagcttcatagtttagcctatagccaactttgcctcaagacctccagataagatgaagtggcttttacctcatgtgagtctcaaacgctggagagagagttccacaaccacccctgccctttctcccatattacgctcattagaaagtctactcagggctgggtgccgtggctcatgcctgtaatcccagcactttggaggctgaggcgggtggatcatctgaggtcaggagttcgagaccagcctggccaacatggggaaaccctgtctctactgaaaatataaaaattagccgggcatggtggcgtgcacctgtaattccagctactcgggaggctgaggcaggagaatcacttgaaccccagaggcagaggctacagtgagccaagattgcaccactgcactccaacctgggtgatagaataagactcttgtctcaaggaaagaaaaaatgtctactcagtagagcagtgattggcaaagtgtgagaccccagaccggcaatattggcatctcctggagacttgtttaaaatgctgattactattttcaagaactttctaccttcatttttacttattttttgttttcaagggctcatggaatcagtcttccatgtgacagctttttaaatattttaagacaaaaactgagtcttctaatcttaattgtgttctagcagtagtcattgtaattatagttatttttataatttatttgtttagtgtatatctgtcactctagaatgtaggctccctgagataaaaactataattcttttgatggcagttacattcccaactcttagcacaagatctaggacatagtatgaactcaataaatttttgttgaatgaaagaaaaatgcaaatttataagccttttcagagacctactgaatcaaactctgagagtggagcccagcaatctgttgtaaaagctgtttgagtaatactgttgcactctaagcttgagaaccactgcaatagaactaagagaacattcatttcaccttcaggcctattctctgacatcacttgaaaaatcctctctgccttctgtaacccttaaccaacaacagtgccctgtccatgtaggggaaacttctgggctgtattacaagtgggtaggaaggtataaggatatcatagtgggaagtattgcttagaatgccagtacttgtttgctgtggaaggtttctaggaatggattgcttagtctaaagtaagcactaccaccatccacgttttcactcaacacagtgccttttaattggtgactaacatcaaaacaccttgacaaaccgttaaaacagtatcagaaaccatgtgtacatccctgtgcctcttagattgctaagttctgctctcattcttacttcaaaggacactttttaaagaattgttaatgcttttttataatacaagtcatagccctgttggcaaaaagaaaaaagattcagtgagaaagaaaaatcactgataattccatcatccagggagaaccactgttatttaagtatatggccttccagacttttctgtgcatttatagatatttttaatgggatcaaagtgtacatactgttttgtaactttttttcactcaacaataccatgactgtcttgctgtgtcaagtatatttctacatctcttaatggctgcatgatattccatcatatgtgccacattttattaaactactgttcaatttttaggtttccaactttcattaataaagtgctttaaagaacaaaaaa

SEQ ID NO: 11 - DNA, 24 bp, Artificial Sequence
  Description of Artificial Sequence Synthetic primer
tgcctcagatgaccaccagccacc

SEQ ID NO: 12 - DNA, 21 bp, Artificial Sequence
  Description of Artificial Sequence Synthetic primer
tcaccactgggtggagccgct

SEQ ID NO: 13 - DNA, 22 bp, Artificial Sequence
  Description of Artificial Sequence Synthetic primer
cttagtgagctgtcaggagagc

SEQ ID NO: 14 - DNA, 22 bp, Artificial Sequence
  Description of Artificial Sequence Synthetic primer
aggttctcatgggaacagcgac

SEQ ID NO: 15 - DNA, 20 bp, Artificial Sequence
  Description of Artificial Sequence Synthetic primer
tcgccttcttgacgagttct

SEQ ID NO: 16 - DNA, 58 bp, Artificial Sequence
  Description of Artificial Sequence Synthetic oligonucleotide
ccggcctacttagaacttctcaattctcgagaattgagaagttctaagtaggtttttg

SEQ ID NO: 17 - DNA, 25 bp, Artificial Sequence
  Description of Artificial Sequence Synthetic primer
attcaaacatcagccttaaatttcc

SEQ ID NO: 18 - DNA, 24 bp, Artificial Sequence
  Description of Artificial Sequence Synthetic primer
aagtcatctgggcttctacaaagt

SEQ ID NO: 19 - DNA, 25 bp, Artificial Sequence
  Description of Artificial Sequence Synthetic primer
attcaaacgtcagccttaaattttc

SEQ ID NO: 20 - DNA, 1122 bp, Artificial Sequence
  Description of Artificial Sequence Synthetic polynucleotide
atgaacatcatggatttcaacgtgaagaagctggcggccgacgcgggcaccttcctcagccgggccgtgcagttcacagaagaaaagcttggccaggcagagaagacagaactggacgctcacctggaaaacctccttagcaaagctgaatgtaccaaaatatggacagaaaagataatgaagcagaccgaagtgctgttgcagccaaatccaaatgccaggatagaagaatttgtttatgagaaactggatagaaaagcaccaagtcgtataaacaacccggaacttttgggacaatatatgattgatgcaggaactgagtttggcccagggacagcttatggaaatgcccttattaaatgtggagaaacacagaagcgaattggaacagctgaccgagagctgattcaaacatcagccttaaatttcctcactcctttaagaaactttatagaaggggattacaaaacaatcgcaaaagaaaggaaactattacagaataagagactggatttggatgctgcaaaaacaagactaaaaaaggcaaaagctgcagaaactaaaagttcaatttgggcagaggaagtgacaaaatctgaacaggaattgagaataactcaaagtgaatttgatcgtcaggcagagattacccgactcctgcttgagggaatcagcagtacacacgcccatcatctccgctgtctgaatgactttgtagaagcccagatgacttactatgcacagtgttaccagtatatgctagacctacagaagcaactgggaagttttccatccaattatctttctaacaacaatcagacctctgggacaccagtgccatatgctttgtcaaatgcaattggtccttctgcccaggcttcaacgggtagccttgtaatcacctgtccttctaacctcaatgaccttaaagaatccagcaacaacaggaaggctagggtcctctatgattatgatgctgcaaatagcactgaactgtcactcctggccgatgaggtaatcactgtgttcagtgtcgttggaatggactccgactggctaatgggagagagaggaaatcaaaagggcaaggtgccaattacctacttagaacttctcaattaa

SEQ ID NO: 21 - DNA, 23 bp, Artificial Sequence
  Description of Artificial Sequence Synthetic oligonucleotide
aannnnnnnnnnnnnnnnnnntt